(19) United States
(12) Patent Application Publication
(10) Pub. No.: US 2025/0325052 A1
(43) Pub. Date: Oct. 23, 2025

(54) Personal Air Management Methods and Systems for Reducing or Blocking Exposure to Airborne Pathogens

(71) Applicants: Virender K. Sharma, Paradise Valley, AZ (US); Kunal M. Sharma, Paradise Valley, AZ (US)

(72) Inventors: Virender K. Sharma, Paradise Valley, AZ (US); Kunal M. Sharma, Paradise Valley, AZ (US)

(21) Appl. No.: 19/189,042

(22) Filed: Apr. 24, 2025

Related U.S. Application Data
(63) Continuation of application No. 17/337,075, filed on Jun. 2, 2021.
Application No. 17/337,075 is a continuation-in-part of application No. 17/336,896, filed on Jun. 2, 2021.
(60) Provisional application No. 63/033,753, filed on Jun. 2, 2020.
(60) Provisional application No. 63/033,753, filed on Jun. 2, 2020.
(60) Provisional application No. 63/062,591, filed on Aug. 7, 2020.
(60) Provisional application No. 63/062,591, filed on Aug. 7, 2020.
(60) Provisional application No. 63/152,267, filed on Feb. 22, 2021.
(60) Provisional application No. 63/152,267, filed on Feb. 22, 2021.
(60) Provisional application No. 63/173,131, filed on Apr. 9, 2021.
(60) Provisional application No. 63/173,131, filed on Apr. 9, 2021.

(51) Int. Cl. A41D 13/11; A61M 16/00; A61M 16/20

(52) U.S. Cl. CPC A41D 13/1146; A41D 13/1161; A61M 16/0087; A61M 16/20; A41D 2500/30

(57) ABSTRACT

The present specification, in some embodiments, describes a personal wear device that direct the flow of air away from a person's face, reducing or blocking the flow of infectious pathogens towards a patient's naso-oral area thus reducing the risk of inhalation of infectious or noxious pathogens. In another embodiment, the present specification describes a personal air management mask system for use by a patient for reducing or preventing exposure to and inhalation of infected aerosol during a medical procedure.

### CROSS-REFERENCE

The present application is a continuation application of U.S. patent application Ser. No. 17/337,075, titled “Personal Air Management Methods and Systems for Reducing or Blocking Exposure to Airborne Pathogens” and filed on Jun. 2, 2021, which is a continuation-in-part application of U.S. patent application Ser. No. 17/336,896, titled “Methods and Systems for Air Management to Reduce or Block Exposure to Airborne Pathogens” and filed on Jun. 2, 2021, which relies on, for priority, United States Patent Provisional Application No. 63/033,753, of the same title and filed on Jun. 2, 2020, United States Patent Provisional Application No. 63/062,591, of the same title and filed on Aug. 7, 2020, United States Patent Provisional Application No. 63/152,267, of the same title and filed on Feb. 22, 2021, and United States Patent Provisional Application No. 63/173,131, of the same title and filed on Apr. 9, 2021.

U.S. patent application Ser. No. 17/337,075 also relies on United States Patent Provisional Application No. 63/033,753, titled “Methods and Systems for Air Management to Reduce or Block Exposure to Airborne Pathogens”, filed on Jun. 2, 2020, for priority.

U.S. patent application Ser. No. 17/337,075 also relies on United States Patent Provisional Application No. 63/062,591 titled “Methods and Systems for Air Management to Reduce or Block Exposure to Airborne Pathogens”, filed on Aug. 7, 2020, for priority.

U.S. patent application Ser. No. 17/337,075 also relies on United States Patent Provisional Application No. 63/152,267, titled “Methods and Systems for Air Management to Reduce or Block Exposure to Airborne Pathogens” and filed on Feb. 22, 2021, for priority.

U.S. patent application Ser. No. 17/337,075 also relies on United States Patent Provisional Application No. 63/173,131 titled “Methods and Systems for Air Management to Reduce or Block Exposure to Airborne Pathogens”, filed on Apr. 9, 2021, for priority.

The above-mentioned applications are herein incorporated by reference in their entirety.

### FIELD

The present specification is related generally to the field of airborne pathogen and infection management. More specifically, the present specification is related to personal wear devices that direct the flow of air away from a person's face and reducing or blocking the flow of infectious pathogens towards a patient's naso-oral area thus reducing the risk of inhalation of infectious or noxious pathogens.

### BACKGROUND

Reducing airborne infections may be accomplished by reducing or killing infectious agents carried in the air and/or by effective air exposure and air quality management. It is common practice in surgical settings and when dealing with infectious disease to manage air quality. Methods include filtration, where the pore size of the filter is smaller than the pathogen, exposure to short wavelength ultraviolet-c light, and by generating ozone and with other chemicals. The air must be breathable after the treatment process. Pathogens include viruses, bacteria, spores, yeast, mold, fungi and other biohazards. Of current interest is improving air quality, via a personal air management system, to curb transmission of, among other viruses, coronaviruses and, in particular, SARS-COV-2 which is the virus responsible for causing COVID-19 in human patients and animals. According to the United States Center for Disease Control, the incubation period is estimated at approximately 5 days, with a wider range of 2-14 days being possible. Frequently reported signs and symptoms include fever, cough, fatigue or myalgia, and shortness of breath. Less commonly reported symptoms include sputum production, headache, hemoptysis, and diarrhea. Some patients have experienced gastrointestinal symptoms such as diarrhea and nausea prior to developing fever and lower respiratory tract signs and symptoms. For certain populations, particularly patients who are 60 years old and older, COVID-19 can be fatal, with mortality rates among certain populations being as high as 20%.

Typical viral particle size ranges from 0.05 to 0.2 microns for coronavirus, 0.5 microns for bacillus, ranges from 0.3 microns to 2 microns for tuberculosis, ranges from 1 to 4 microns for anthrax, and up to 1 micron for black mold spores. Good filters (HEPA, tight fitting masks, etc.) filter out large particles and 95% of particles as small as 0.3 micron. Filter masks are effective for tuberculosis and other bacterial infections. They are less effective for viruses which are 10 times smaller in diameter than most bacteria. Extremely fine mesh filters also have pressure drops that necessitate a pump to assist the airflow. Systems with filters and pumps are Powered Air Purifying Respirators (PAPR). Using a high efficiency (HE) filter they claim removal of 99.97% of 0.3 mm particles (laboratory testing).

In a study published in 2013, data was collected using a non-invasive, visualization approach to the airflow dynamics of sneezing and breathing in healthy human volunteers. The study also made a direct comparison between maximum cough and sneeze velocities using a shadowgraph method, which, surprisingly, shows them to be firstly, quite similar in speed, and secondly, that this speed is not extremely high, as has been inferred in some older estimates of sneeze velocity. FIG. 1, FIG. 2, FIG. 3, and FIG. 4 show results of the 2013 study.

FIG. 1 shows two graphs depicting mouth breathing air flow parameters for potential particle transmission. Graph 101 shows a time vs. visible propagation distance and time vs. velocity plot, correlating the time it takes for air and/or airborne particles to travel a distance when propagated from a person's mouth and the velocity at which such air flows. Graph 102 shows a time versus 2D projected area plot and a time versus 2D projected area expansion rate plot showing the time and velocity it takes for air to flow from a person's mouth to propagate into a 2D projected area.

FIG. 2 shows two graphs depicting nasal breathing air flow parameters for potential particle transmission. Graph 201 shows a time vs. visible propagation distance and time vs. velocity plot, correlating the time it takes for air and/or airborne particles to travel a distance when propagated from a person's nose and the velocity at which such air flows. Graph 202 shows a time versus 2D projected area plot and a time versus 2D projected area expansion rate plot showing the time and velocity it takes for air to flow from a person's nose to propagate into a 2D projected area.

FIG. 3 shows two graphs depicting sneezing air flow parameters for potential particle transmission. Graph 301 shows a time vs. visible propagation distance and time vs. velocity plot, correlating the time it takes for air and/or airborne particles to travel a distance when propagated from a person's sneeze and the velocity at which such air flows. Graph 302 shows a time versus 2D projected area plot and a time versus 2D projected area expansion rate plot showing the time and velocity it takes for air to flow from a person's sneeze to propagate into a 2D projected area.

FIG. 4 shows two graphs depicting coughing air flow parameters for potential particle transmission. Graph 401 shows a time vs. visible propagation distance and time vs. velocity plot, correlating the time it takes for air and/or airborne particles to travel a distance when propagated from a person's cough and the velocity at which such air flows. Graph 402 shows a time versus 2D projected area plot and a time versus 2D projected area expansion rate plot showing the time and velocity it takes for air to flow from a person's cough to propagate into a 2D projected area.

It should be noted that many conventional devices, such as wearable fans or oxygen supply masks are optimized to have the majority of air move towards a person, rather than away.

Therefore, what is needed is a wearable personal air quality management system that creates an air flow of filtered air, creating an air shield, to reduce or prevent exposure to and inhalation of infected aerosol by redirecting infected aerosol away from a healthy person preventing transmission of airborne infection to the person wearing the system.

What is also needed is a personal air delivery device that is wearable, adjustable, and causes the majority of air to move away from a person rather than towards or onto a person, creating an air shield.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a face mask configured to cover a nose and a mouth of a user, wherein the face mask comprises: a transparent surface configured to flexibly seal against a skin surface of the user, wherein the skin surface is positioned over at least a portion of the user's zygomaticus major muscle, at least a portion of the user's zygomaticus minor muscle, all of the user's orbicularis oris muscle, at least a portion of the user's risorius muscle, all of the user's depressor muscle, and all of the user's mentalis muscle, wherein the transparent surface is further configured to be positioned at least a distance of +/−10 mm from a junction of the bony and cartilaginous part of the nose and under a chin between a tip of the chin to a junction of a bottom of a face and a neck of the user, and wherein the transparent surface is non-porous; a first non-transparent filtration area, wherein the first non-transparent filtration area is configured to removably receive a first filter material, wherein the first non-transparent filtration area has a surface area in a range of 4.5 square inches to 7 square inches, and is positioned to at least partially cover the user's buccinator, masseter, zygomaticus major, and risorius muscles on a left side of the user's face; a second non-transparent filtration area, wherein the second non-transparent filtration area is configured to removably receive a second filter material, wherein the second non-transparent filtration area has a surface area in a range of 4.5 square inches to 7 square inches, and is positioned to at least partially cover the user's buccinator, masseter, zygomaticus major, and risorius muscles on a right side of the user's face, wherein, when the face mask is worn by the user, the first non-transparent filtration area and the second non-transparent filtration area are adapted to reduce inhaled bioaerosol of less than 10 microns by more than 33%; and at least one strap configured to secure the face mask in place.

Optionally, at least one of the first non-transparent filtration area with the first filter material or the second non-transparent filtration area with the second filter material is configured to have a filtration efficiency of greater than or equal to 95%. Optionally, the filtration efficiency of greater than or equal to 95% is against a sodium chloride (NaCl) aerosol challenge with a count median diameter of 75±20 nm and a geometric standard deviation of 1.86 and an inhalation airflow resistance of ≤15 mm H₂O at a flowrate of 85±4 LPM and at a face velocity of 10 cm/sec.

Optionally, at least one of the first filter material or the second filter material comprises non-woven fabric.

Optionally, the first non-transparent filtration area and the second non-transparent filtration area, in combination, are less than 75% of an entire surface area of the face mask.

Optionally, an entire area of the transparent surface is more than 25% of an entire surface area of the face mask.

Optionally, the transparent surface comprises a first material and a perimeter of the transparent surface comprises a second material.

Optionally, the second material has a durometer rating that is less than a durometer rating of the first material.

Optionally, the face mask is configured to fit more than 50% of face structures across two different ISO digital head forms created using NIOSH anthropomorphic data.

Optionally, the face mask further comprises a third non-transparent filtration area positioned at a base of the face mask and positioned to be proximate a chin of the user.

Optionally, at least one of the first non-transparent filtration area or the second non-transparent filtration area comprises a closed-ring sealing material extending around a periphery of the first non-transparent filtration area or the second non-transparent filtration area.

Optionally, at least one of the first non-transparent filtration area or the second non-transparent filtration area is configured to receive a replaceable filter and wherein the replaceable filter is adapted to house at least one of the first filter material or second filter material. Optionally, the replaceable filter is configured to magnetically attach to at least one of the first non-transparent filtration area or the second non-transparent filtration area. Optionally, the face mask further comprises a cover configured to attach at least one of the first non-transparent filtration area or the second non-transparent filtration area and encase the replaceable filter.

The present specification also discloses a face mask configured to cover a nose and a mouth of a user, wherein the face mark comprises: a transparent surface configured to flexibly seal against a skin surface of the user, wherein the skin surface is positioned over at least a portion of the user's zygomaticus major muscle, at least a portion of the user's zygomaticus minor muscle, all of the user's orbicularis oris muscle, at least a portion of the user's risorius muscle, all of the user's depressor muscle, and all of the user's mentalis muscle, wherein the transparent surface is further configured to be positioned at least a distance of +/−10 mm from a junction of the bony and cartilaginous parts of the nose] and under a chin between a tip of the chin to a junction of a bottom of a face and a neck of the user, and wherein the transparent surface is non-porous; a first non-transparent port on a left side of the face mask, wherein the first air non-transparent port has a surface area in a range of 4.5 square inches to 7 square inches, is positioned over at least a portion of the user's buccinator, masseter, zygomaticus major, and risorius muscles, and is configured to removably receive a filter material; a second non-transparent port on a right side of the face mask, wherein the second non-transparent port has a surface area in a range of 4.5 square inches to 7 square inches, is positioned over at least a portion of the user's buccinator, masseter, zygomaticus major, and risorius muscles, and is configured to removably receive a filter material; a non-transparent oral valve, wherein the non-transparent oral valve has a surface area in a range of 75 square mm to 700 square mm, is positioned over at least a portion of the user's mouth, and is configured to removably receive an endoscope; and an air input port, wherein the air input port is positioned over at least a portion of the user's chin, and is configured to removably connect to an oxygen source.

Optionally, the oral valve comprises a two-way diaphragm valve.

Optionally, the oral valve comprises a one-way flap valve adapted to prevent aerosolized oropharyngeal content from escaping the face mask.

Optionally, the air input port comprises a quick connect port connection.

Optionally, the air input port comprises an inlet valve and a filter housing.

Optionally, the transparent surface comprises a first material and wherein a perimeter of the transparent surface comprises an inflatable bladder configured to have a non-inflated state and to have an inflated state. Optionally, the first material is silicone, and the inflatable bladder comprises a material different than silicone. Optionally, the face mask further comprises a port configured to attach to the inflatable bladder and through which fluid may be provided to the inflatable bladder to change the inflatable bladder from the non-inflated state to the inflated state. Optionally, the inflation port is positioned proximate the user's chin.

Optionally, a rim of the transparent surface comprises a silicone rubber material.

Optionally, each of the first non-transparent port, the second non-transparent port, the non-transparent oral valve, and the air input port comprise filter material.

Optionally, at least one of the first non-transparent port, the second non-transparent port, the non-transparent oral valve, or the air input port comprise filter material.

Optionally, the non-transparent oral valve comprises a droplet control sleeve comprising an opening ranging from 2 mm and configured to expand up to 36 mm to receive a shaft of an endoscope.

The present specification also discloses a wearable face structure comprising: an air filtration system; a pump integrated with the air filtration system; and air delivery ports in communication with the air filtration system, to deliver filtered air towards the face, wherein the wearable structure reduces inhaled bioaerosol of less than 10 microns by more than 33%.

Optionally, the wearable face structure does not form or require an air tight seal around a patients nose or mouth like a mask.

Optionally, the air delivery ports drive unfiltered air away from the face.

Optionally, the air delivery ports deliver filtered air in at least one of: a downward direction, a downward and outward, an upward direction, an upward and outward direction, a front direction, a front inward direction, and a sideward direction, wherein each direction is relative to the face.

Optionally, the air filtration system is positioned remote from the air delivery ports, wherein the air delivery ports are in proximity to a face of a wearer of the wearable face structure.

Optionally, the wearable face structure further comprises a transparent face shield, wherein the air delivery ports are configured to deliver filtered air within the transparent face shield.

Optionally, the air filtration system performs mechanical filtration.

Optionally, the air filtration system performs destructive filtration using ultraviolet radiation and ionization.

Optionally, the air filtration system performs filtration using centrifugal, non-laminar air flow.

Optionally, the air filtration system weighs less than 5 lbs.

Optionally, the wearable structure has a noise level of less than 65 decibel during operation.

Optionally, the wearable structure weighs less than 5 lbs.

Optionally, the air delivery ports cover less than 95% of a surface of the wearable structure.

Optionally, the wearable structure has a delivery rate of >30 LPM of filtered air.

The present specification also discloses a mask wearable by a patient, to perform a procedure through the mouth or nose of the patient, wherein the mask comprises: a first opening to enable a medical instrument to pass through the first opening; a second opening to apply suction to the mask wherein the suction is between 25 mm Hg and 500 mm Hg and wherein the application of suction reduces a number of respiratory droplets released by the patient by at least 10%; a filtration surface with a first area; and a flexible seal on a perimeter of the face mask.

Optionally, the mask is comprised of a material with durometer rating of less than 90.

Optionally, the flexible seal fits more than 50% of face structures across two different ISO digital head forms created using NIOSH anthropomorphic data.

Optionally, the first area is in a range of 25% to 90% of a surface area of the mask.

Optionally, the mask comprises a transparent surface area that is in a range of 25% to 90% of a surface area of the mask.

Optionally, the mask reduces inhaled bioaerosol of less than 10 microns by more than 25% over a minute of sampling.

The present specification also discloses a face mask comprising: a filtration surface with a first area; a non-filtration surface with a second area less than the first area; and a flexible seal on a perimeter of the face mask; wherein wearing the face mask reduces inhaled bioaerosol of less than 10 microns by more than 33%.

Optionally, the filtration surface has a filtration efficiency ≥95% against a sodium chloride (NaCl) aerosol challenge with a count median diameter of 75±20 nm and a geometric standard deviation of 1.86 and an inhalation airflow resistance of ≤15 mm H2O at a flowrate of 85±4 LPM and at a face velocity of 10 cm/sec.

Optionally, the filtration area is less than 75% of a surface of the face mask.

Optionally, the non-filtration surface area is more than 25% of a surface of the face mask.

Optionally, the flexible seal fits more than 50% of face structures across two different ISO digital head forms created using NIOSH anthropomorphic data.

Optionally, the filtration surface is distributed across one of two and three openings in the face mask.

Optionally, the filtration surface is fitted with replaceable filters. Optionally, a perimeter of each replaceable filter comprises an O-ring seal.

Optionally, the filtration surface comprises an insert to support a replaceable filter, wherein the insert spreads over an outer surface of the replaceable filter. Optionally, the insert is magnetically attached to the face mask.

Optionally, the filtration surface comprises a cover to support the replaceable filter, wherein the cover spreads over an inner surface of the replaceable filter.

Optionally, the face mask is transparent.

The present specification also discloses a wearable face structure comprising: an air filtration system; a pump integrated with the air filtration system; and air delivery ports in communication with the air filtration system, to deliver filtered air towards the face, wherein the wearable structure reduces inhaled bioaerosol of less than 10 microns by more than 33%.

Optionally, the air delivery ports drive unfiltered air away from the face.

Optionally, the air delivery ports deliver filtered air in at least one of: a downward direction, a downward and outward, an upward direction, an upward and outward direction, a front direction, a front inward direction, and a sideward direction, wherein each direction is relative to the face.

Optionally, the air filtration system is positioned remote from the air delivery ports, wherein the air delivery ports are in proximity to a face of a wearer of the wearable face structure.

Optionally, the wearable face structure further comprises a transparent face shield, wherein the air delivery ports are configured to deliver filtered air within the transparent face shield.

Optionally, the air filtration system performs mechanical filtration.

Optionally, the air filtration system performs destructive filtration using ultraviolet radiation and ionization.

Optionally, the air filtration system performs filtration using centrifugal, non-laminar air flow.

Optionally, the air filtration system weighs less than 5 lbs.

Optionally, the wearable structure has a noise level of less than 65 decibel during operation.

Optionally, the wearable structure weighs less than 5 lbs.

Optionally, the air delivery ports cover less than 95% of a surface of the wearable structure.

Optionally, the wearable structure has a delivery rate of >30 LPM of filtered air.

The present specification also discloses a mask wearable by a patient, to perform a procedure through the mouth or nose of the patient, wherein the mask comprises: a first opening to enable a medical instrument to pass through the first opening; a second opening to apply suction to the mask wherein the suction is between 25 mm Hg and 500 mm Hg and wherein the application of suction reduces a number of respiratory droplets released by the patient into the surroundings or the procedure room by at least 10%; a filtration surface with a first area; and a flexible seal on a perimeter of the face mask.

Optionally, the mask is comprised of a material with durometer rating of less than 90.

Optionally, the flexible seal fits more than 50% of face structures across two different ISO digital head forms created using NIOSH anthropomorphic data.

Optionally, the first area is in a range of 25% to 90% of a surface area of the mask.

Optionally, the mask comprises a transparent surface area that is in a range of 25% to 90% of a surface area of the mask.

Optionally, the mask reduces inhaled bioaerosol of less than 10 microns by more than 25% over a minute of sampling.

In some embodiments, the present specification discloses a personal air management system for reducing or preventing exposure to and inhalation of infected aerosol, comprising: an air inlet; a high-flow, high pressure air pump, wherein said pump operates with an air flow ranging from 10 to 500 L/min and a pressure less than or equal to 50 psi; a particulate filter; an mechanism for destructive filtration; an optional air tubing; and, a personal wearable appliance.

Optionally, the mechanism for destructive filtration is UV-C/photocatalytic oxidation (PCO)/photoelectrochemical oxidation (PECO)/negative or bipolar plasma ionization or an antimicrobial filter (e.g. incorporated with silver, zinc or another antimicrobial coating).

Optionally, the UV filter is a UV-C filter, wherein the wavelength of the UV filter ranges from 200-280 nm.

Optionally, the antimicrobial filter is a hydrophobic PES, PTFE, glass microfiber membrane, or HEPA filter with antimicrobial coating that is optionally housed in a cardboard, polypropylene or styrene housing.

Optionally, the air pump includes a battery.

Optionally, the air pump has dimensions ranging from 1 inch to 4 inches.

Optionally, the personal facial appliance is a mask or face shield or a wearable structure worn proximate a person's nose or mouth and provide clean air for inhalation.

Optionally, the air tubing includes at least one port at a first end in fluid communication to the air pump and an opening at a second end for receiving or delivering air.

Optionally, the system further comprises a soft nasal plug.

Optionally, the particulate filter comprises a hydrophilic PES, PTFE, glass microfiber membrane or nylon filter.

In some embodiments, the system contain an inlet filter rated less than or equal to MERV 11 and an outlet filter rated greater than or equal to MERV 11.

In some embodiments, the present specification is directed towards a method for providing filtered air to an individual while reducing or preventing exposure to and inhalation of infected aerosol, comprising: receiving, via an air inlet, atmospheric air; directing the air through a high-flow, high pressure pump, wherein said pump operates with an air flow ranging from 10 to 500 L/min and a pressure less than or equal to 50 psi; passing the air through one or more particulate filter; passing the air through an optional humidification chamber; passing the air through an destructive filtration mechanism; and, delivering a first portion of the air to the individual for inhalation via a personal appliance while circulating a second portion of air away from the individual using the personal appliance and the individual's exhaled breath, creating a clean air space of a volume of 1 L or more surrounding a person's nose and mouth or an “air shield”.

Optionally, the second portion of air is greater than the first portion.

In some embodiments, the present specification is directed towards a personal air management mask system for use by a patient for reducing or preventing exposure to and inhalation of infected aerosol during a medical procedure, comprising: at least one air inlet; at least one air outlet, wherein the at least one air outlet is positioned away from the air inlet; and, a detachable tubing in fluid communication with air outlet.

Optionally, the at least one air inlet includes a valve. Optionally, the valve is a one-way valve.

Optionally, the at least one air inlet includes a filter.

Optionally, the oral opening includes a two-way diaphragm valve.

Optionally, the oral opening includes a one-way flap valve.

Optionally, the system further comprises at least one port for attaching a tubing to provide oxygen to a patient, wherein the tubing may be connected to an air pump or wall oxygen.

Optionally, the system further comprises an inflation port integrated with an inflatable rim for providing comfort to the patient.

Optionally, the oral opening is used to insert an endoscope and wherein the oral opening expands with the passage of the endoscope while ensuring no aerosol leaks.

Optionally, the system further comprises a rebreather bag.

In some embodiments, the present specification is directed towards an air management system for reducing or preventing exposure to and inhalation of infected aerosol to individuals in a room, comprising: an air inlet; a dust filter; a high-flow air pump, wherein said pump operates with an air flow ranging from 10 cfm to upwards of 20 cfm; a UV light source, encircled by a non-linear hollow tube air pathway connected to the air inlet, wherein the UV light source is a germicidal light; a HEPA filter; and, an air outlet.

Optionally, the capacity of the system ranges from 40 gallons to 100 gallons.

Optionally, the hollow tube pathway is fabricated from clear quartz and includes a plurality of bends or turns, forming a coil around the UV light source.

Optionally, air that is received, via the air inlet and through the hollow tube pathway, is exposed to UV light for at least one second, and preferably for a suitable time period to effectuate antimicrobial/viral inactivation.

Optionally, the non-linear hollow tube pathway includes hollow quartz balls to create turbulence and increase the path of air flow.

Optionally, the system further comprises housing the hollow tube pathway and UV light source within an enclosure.

The aforementioned and other embodiments of the present specification shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1 is a graph showing mouth breathing air flow parameters;

FIG. 2 is a graph showing nasal breathing air flow parameters;

FIG. 3 is a graph showing sneezing air flow parameters;

FIG. 4 is a graph showing coughing air flow parameters for comparison;

FIG. 5 is a schematic diagram showing air flow to an individual both with and without an air shield, in accordance with some embodiments of the present specification;

FIG. 6 is a block flow diagram showing the operational flow of a personal air management system in accordance with some embodiments of the present specification;

FIG. 7 is a schematic diagram showing air flow both with and without use of a personal air management system in accordance with various embodiments of the present specification;

FIG. 8 is a schematic diagram showing air flow both with and without use of a personal air management system in accordance with various embodiments of the present specification;

FIG. 9 is an illustration of an exemplary neck band that is designed for use with embodiments of the present specification;

FIG. 10 is an illustration of a neck band that includes an integrated air filtration system;

FIG. 11 is a schematic diagram showing air flow both with and without use of a personal air management system in accordance with various embodiments of the present specification;

FIG. 12 is a schematic diagram showing air flow both with and without use of a personal air management system in accordance with various embodiments of the present specification;

FIG. 13 is an illustration showing a headband that creates an air shield around the user's head where the air flow is directed downwards in a “shower” flow pattern;

FIG. 14 is an illustration showing an eye shield or face shield that may be used with embodiments of the present specification to create an air shield around the face and head of a user;

FIG. 15 is an illustration showing an eye shield or face shield that may be used with embodiments of the present specification to create an air shield around the face and head of a user;

FIG. 16 illustrates an embodiment of a personal air filtration system (PAFS) that may be used, in accordance with some embodiments of the present specification;

FIG. 17 illustrates embodiments of a neck wearable PA FS in accordance with some embodiments of the present specification;

FIG. 18 illustrates multiple embodiments of PAFS, in accordance with the present specification;

FIG. 19 illustrates an embodiment of a blower that may be used with devices in accordance with the present specification;

FIG. 20 illustrates a front perspective view, a side view, and a rear view of a mask with openings, in accordance with some embodiments of the present specification;

FIG. 21A shows different views of a silicone mask body with an insert used for placing a filter in accordance with some embodiments of the present specification;

FIG. 21B illustrates front elevation and top plan views of a mask body with openings/port for inserting filters;

FIG. 21C illustrates front elevation and top plan views of a mask body with an alternate openings/port design for inserting filters;

FIG. 21D is a side elevation view of yet another design of an insert configured on a mask body in a side view of the mask;

FIG. 21E illustrates exemplary embodiments of inserts showing their dimensions, in accordance with some embodiments of the present specification;

FIG. 21F shows exemplary configurations of an aluminum wire used with the inserts of the present specification;

FIG. 22A is a side elevation view of an exemplary design of an insert over-molded with a snap fit attachment to cover an internal surface of a filter to support the filter that is placed between the insert and the cover;

FIG. 22B is a side elevation view of the layers created by a cover, a filter, and an insert, in a mask body, in accordance with some embodiments of the present specification;

FIG. 22C is a perspective view of an exemplary configuration of a cover with a snap fit attachment, in accordance with some embodiments of the present specification;

FIG. 22D is a schematic diagram showing a process of assembling a filter in a mock-up of a cross-section of a mask body, in accordance with some embodiments of the present specification;

FIG. 23 illustrates different types of mask configurations in accordance with some embodiments of the present specification;

FIG. 24A illustrates a front top perspective view of a three-port mask in accordance with some embodiments of the present specification;

FIG. 24B illustrates a front perspective view of a three-port mask in accordance with some embodiments of the present specification;

FIG. 24C illustrates a top view of a mask in accordance with some embodiments of the present specification;

FIG. 24D illustrates a seal of a mask in accordance with some embodiments of the present specification;

FIG. 24E illustrates a magnetic insert of a mask in accordance with some embodiments of the present specification;

FIG. 24F illustrates a side perspective view of a mask body with a magnetically attached insert in accordance with some embodiments of the present specification;

FIG. 24G illustrates a front view, a cross-sectional view, and a back view of an insert with magnets of a mask, in accordance with some embodiments of the present specification;

FIG. 24H illustrates a side elevated view and a top view of a magnet of a mask, in accordance with some embodiments of the present specification;

FIG. 24I illustrates a filter with an O-ring seal of a mask in accordance with some embodiments of the present specification;

FIG. 24J illustrates a mask with an insert that includes at least four protrusions from four equally spaced edges of circumference of the circular insert, in accordance with some embodiments of the present specification;

FIG. 24K illustrates various views of a mask with exemplary dimensions of some of the components, in accordance with some embodiments of the present specification;

FIG. 24L illustrates an exemplary wire form of a face mask, in accordance with some embodiments of the present specification;

FIG. 25A illustrates different face sizes and a table showing common face widths in millimeter (mm) that correspond to common face lengths (in mm);

FIG. 25B illustrates an anatomy of a human face;

FIG. 26 illustrates an exemplary front view, a cross sectional view, and a detailed view of an edge of cross-sectional view of a filter, in accordance with some embodiments of the present specification;

FIG. 27A illustrates a break-away view of a mask packaging design, in accordance with some embodiments of the present specification;

FIG. 27B illustrates another break-away view of components within the mask packaging design of FIG. 27A, additionally showing the components of one filter box and its contents;

FIG. 28 illustrates an exploded view of a filter with layers, in accordance with some embodiments of the present specification;

FIG. 29A shows a first view, a second view, and a third view of a positive air pressure (PAP) mask, in accordance with some embodiments of the present specification;

FIG. 29B shows various views of the air intake blower or pump positioned in the at least one air inlet of the mask;

FIG. 30A is an illustration showing different views of a three-port mask in accordance with some embodiments of the present specification;

FIG. 30B is an illustration of a user/patient wearing three-port mask in accordance with some embodiments of the present specification, to prevent airborne pathogen droplet spread;

FIG. 31A is a side elevation diagram of the mask shown in FIG. 30B;

FIG. 31B is a three-dimensional pictorial representation of the mask shown in FIG. 31A;

FIG. 31C is an exploded, perspective view of the mask shown in FIG. 31A;

FIG. 31D is a perspective view of the mask shown in FIG. 31A;

FIG. 31E is a side elevation view of the mask shown in FIG. 31D;

FIG. 31F is a rear elevation view of the mask shown in FIG. 31D;

FIG. 31G is a perspective view of the mask shown in FIG. 31A, showing a low profile oral port in accordance with an embodiment of the present specification;

FIG. 31H is a side elevation view of a mask in accordance with an embodiment of the present specification showing a high profile oral port;

FIG. 31I is a side elevation view of a mask in accordance with a preferred embodiment of the present specification showing a low profile oral port;

FIG. 31J is a perspective bottom view of a mask in accordance with a preferred embodiment of the present specification showing a low profile oral port;

FIG. 31K is a perspective bottom view of a mask in accordance with an embodiment of the present specification, with a quick connect mechanism removed;

FIG. 31L is an exploded, perspective view of the quick connect mechanism shown in FIG. 31J;

FIG. 31M is a side perspective view of the quick connect mechanism shown in FIG. 31L, connected to a hose/tubing, via an optional oxygen port;

FIG. 31N is a top front perspective view of the quick connect mechanism shown in FIG. 31L, connected to an optional oxygen port;

FIG. 31O shows a first perspective view and a second close-up view of the mask shown in FIG. 36A and that is configured for application of suction and to receive a droplet control sleeve, in accordance with some embodiments of the present specification;

FIG. 31P shows first and second views illustrating a plurality of tubing and connections with corresponding ports of the mask of FIG. 31A, in accordance with some embodiments of the present specification;

FIG. 31Q illustrates a soft nasal bridge adjustment that is provided on a top portion of a seal for a mask, in accordance with some embodiments of the present specification;

FIG. 31R illustrates an exploded view of a procedural mask assembled with various components, in accordance with some embodiments of the present specification;

FIG. 31S is a flow chart illustrating an exemplary process of using the procedural mask of FIG. 31R;

FIG. 32 illustrates an embodiment of the oral opening/valve shown in FIG. 31A;

FIG. 33 illustrates another embodiment of the oral opening/valve shown in FIG. 31A;

FIG. 34 illustrates yet another embodiment of the oral opening/valve shown in FIG. 31A;

FIG. 35 illustrates two exemplary air inlets that may be used with embodiments of the present specification as described with respect to FIGS. 30A/30B and 31A;

FIG. 36 is a flow chart describing the steps involved in using and fitting a procedural mask in accordance with the present specification;

FIG. 37A shows front and side views of a person wearing a mask in accordance with some embodiments of the present specification, where the mask includes three quick connection ports for connection to a hosing or tubing for clinical use;

FIG. 37B shows front and side views of a person wearing a mask in accordance with some embodiments of the present specification, where the mask includes a sleeve positioned within an oral/instrument valve opening;

FIG. 37C is a perspective view of a filter housing;

FIG. 37D is a perspective view of a quick connect port with threading;

FIG. 37E is a side view of a person wearing a mask in accordance with some embodiments of the present specification, where the mask includes a quick connect port with a threaded connection to a filter housing as shown in FIGS. 37C and 37D;

FIG. 38 illustrates an exemplary embodiment of an 85 mm mask worn by a user, with a tool inserted through an oral opening of a mask, in accordance with some embodiments;

FIG. 39A is a perspective view of an Ultra-Violet C (UV-C) sterilizer device, in accordance with some embodiments of the present specification;

FIG. 39B is a first side elevation view of the sterilizer device shown in FIG. 39A, with the lid removed and an article positioned within the housing for sterilization;

FIG. 39C is a second side elevation view of the sterilizer device shown in FIG. 39A, with the lid removed and an article positioned within the housing for sterilization;

FIG. 39D shows a lithium ion battery, a micro USB connector and the UV lamp of the sterilizer device attached to a printed circuit board;

FIG. 39E is an internal, perspective view of internal components of a UV-C sterilizer device, in accordance with some embodiments of the present specification;

FIG. 39F is another internal, perspective view of internal components of a UV-C sterilizer device, in accordance with some embodiments of the present specification;

FIG. 39G is a cross-sectional view of internal components of a UV-C sterilizer device, in accordance with some embodiments of the present specification;

FIG. 39H is a side elevation view of a UV-C sterilizer device, showing a power button to operate the UV-C light source, in accordance with some embodiments of the present specification;

FIG. 39I is a flow diagram showing steps for sterilizing an article as shown and described with respect to FIGS. 39A through 39H, in accordance with some embodiments of the present specification;

FIG. 40 is an illustration of an exemplary personal air delivery device that may be used in embodiments of the present specification;

FIG. 41 is an illustration of a low-profile filtered air delivery system and integrated shield;

FIG. 42 is an illustration showing a nasal prong filter (nasal plug of FIG. 41) that may be used in embodiments of the present specification;

FIG. 43 is an illustration showing a nasal prong filter that may be used in embodiments of the present specification;

FIG. 44 is an illustration of a low-profile filtered air delivery system and integrated facial cover as worn by a user/patient;

FIG. 45 is an illustration of the operation and air flow of a low-profile filtered air delivery system, when worn by a user;

FIG. 46A is an illustration of an embodiment of a UV-C anti-microbial filter that may be used in conjunction with the embodiments described in the present specification;

FIG. 46B is a schematic diagram of the filter shown in FIG. 46A encased in a housing; and

FIG. 46C shows various parameters of UV-C sources that may be used with the embodiments described in the present specification.

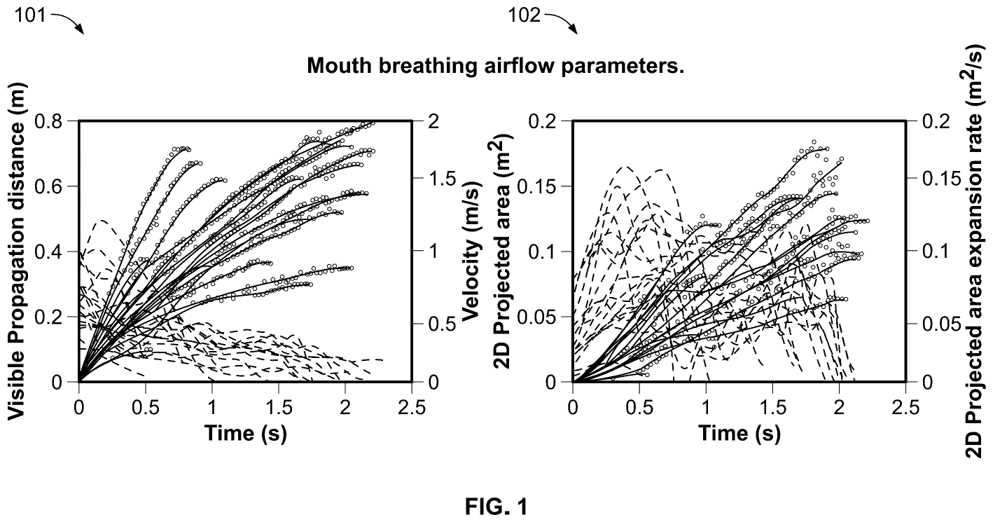

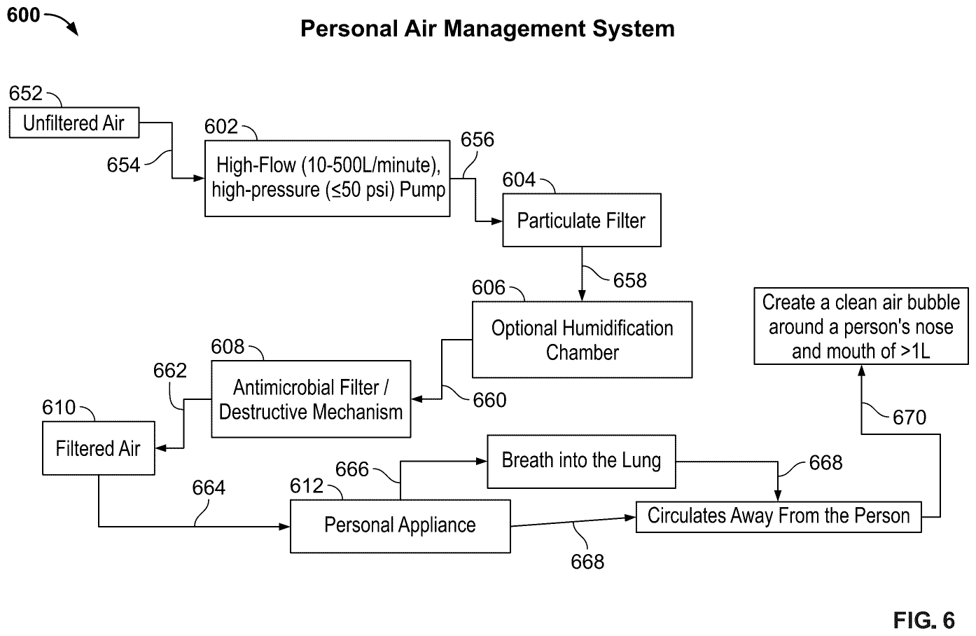

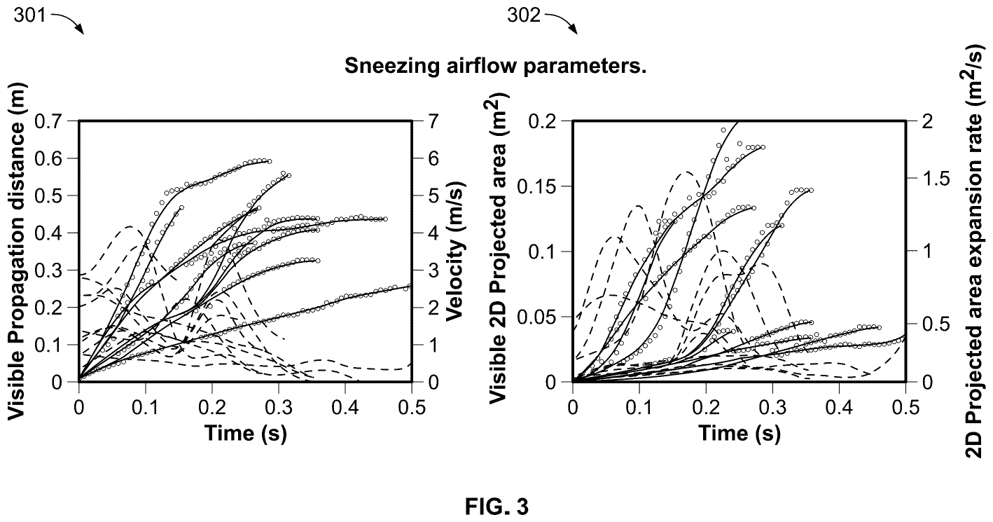

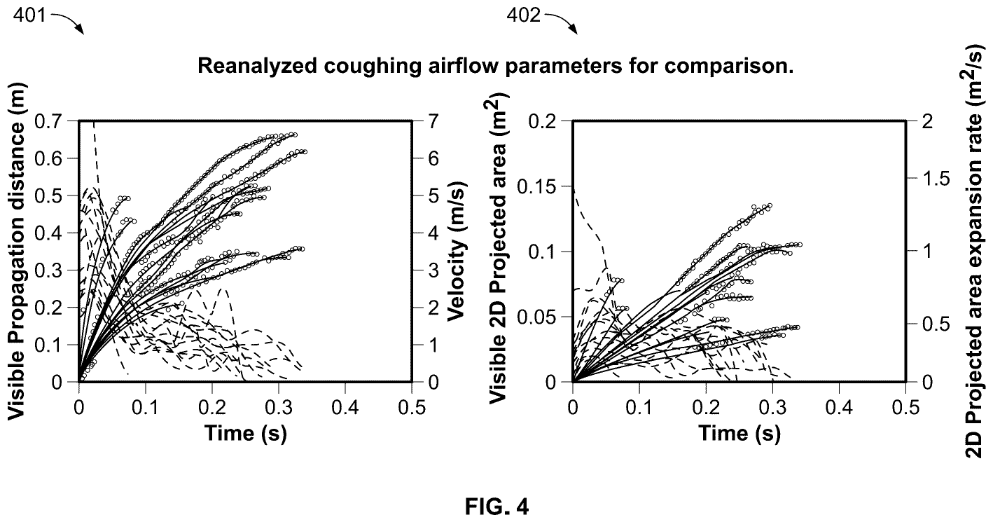

### DETAILED DESCRIPTION

The present specification is directed toward various systems and methods for a wearable personal air quality management. In embodiments, the methods and systems of the present specification are designed to create an air flow of filtered air for a user, and a second flow of air moving away from the user, creating a bubble of clean, nonpathogenic air having a volume of at least 1 Liter, or an air shield around the user's nose and mouth, to reduce or prevent exposure to and inhalation of infected aerosol by redirecting infected aerosol away from a healthy person using a flow of clean filtered air and or exhaled air, or diluting the infected air with clean filtered air and/or exhaled air reducing or preventing spread of airborne infection to the user.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

It should be noted herein that the terms user, patient, and person may be used interchangeably and may be used to refer to an individual using the devices of the present specification.

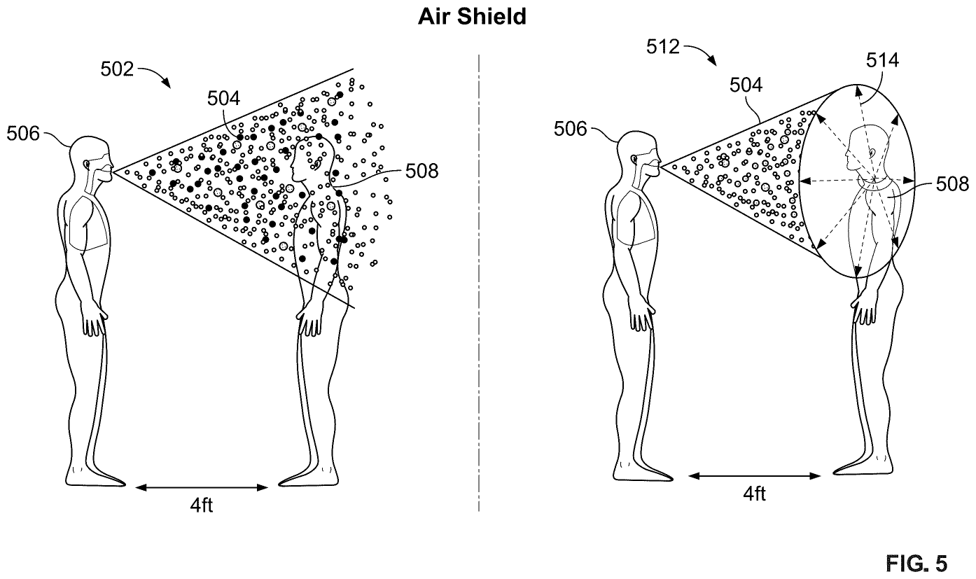

FIG. 5 is a schematic diagram showing air flow both with and without use of a personal air management system in accordance with various embodiments of the present specification. As shown in diagram 502, aerosolized droplets 504 may contain a respiratory pathogen which flows away from a sick person 506 towards a healthy person 508. The healthy person 508 breathes in the pathogenic air, whereby it is inhaled into the healthy person's lungs, infecting the healthy person 508. As shown in diagram 512, various embodiments of the personal air management system create an airflow of filtered air 514 around the healthy person's nose and mouth, pushing the infected aerosol away from the healthy person 508, thus creating an air shield around the healthy person and preventing spread of airborne infection.

Various filters are used in several embodiments throughout the present specification. The United States Environmental Protection Agency sets forth standards for Minimum Efficiency Reporting Values (MERVs) to report a filter's ability to capture larger particles between 0.3 and 10 microns (μm), where the rating is derived from a test method developed by ASHRAE. Table 1 is a table describing various MERV ratings and average particle size efficiency.

TABLE 1

| MERV | Average Particle Size in Microns |
|---|---|
| Rating | (Efficiency Percentage) |
| 1-4 | 3.0-10.0 (less than 20%) |
| 6 | 3.0-10.0 (49.9%) |
| 8 | 3.0-10.0 (84.9%) |
| 10 | 1.0-3.0 (50%-64.9%) |
| | 3.0-10.0 (85% or greater) |
| 12 | 1.0-3.0 (80%-89.9%) |
| | 3.0-10.0 (90% or greater) |
| 14 | 0.3-1.0 (75%-84%) |
| | 1.0-3.0 (90% or greater) |
| 16 | 0.3-1.0 (75% or greater) |

It should be noted that a low efficiency filter, as used in this specification, refers to a filter that holds a MERV rating of less than or equal to 11. It should be noted that a high efficiency filter, as used in this specification, refers to a filter that holds a MERV rating of greater than or equal to 11, and typically includes HEPA and ULPA filters.

### PERSONAL AIR FILTRATION SYSTEM (PAFS)

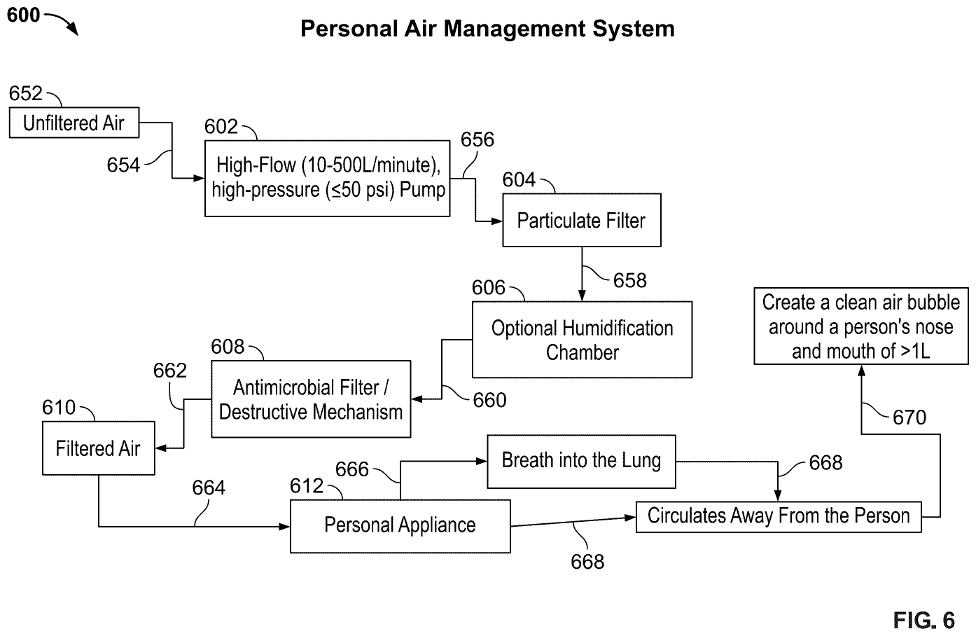

FIG. 6 is a block flow diagram showing exemplary components of an air filtration and management system that can be implemented in various embodiments of the present specification. Personal air management system 600 takes in unfiltered atmospheric air, in step 652, and processes the unfiltered air at step 654 using a high-flow, high pressure pump 602. In embodiments, the pump operates with an air flow ranging from 10 to 500 L/minute. In embodiments, the pump 602 operates at a pressure less than or equal to 50 psi. Once the air passes through the pump 602, in an embodiment, at step 656, the air is passed through, using the pump 602, a particulate filter 604. In some embodiments, the particulate filter 604 is positioned before the air pump 602. After passing through the particulate filter 604, at step 658, the air is passed through an optional humidification chamber 606. Thereafter, at step 660, the air is passed through a destructive filtration mechanism or process or an antimicrobial filter 608, creating filtered air 610 at step 662. The destructive filtration process refers to the destructive ability of the filtration system to destroy bacteria and other airborne pathogens during filtration or by use of a filter. In some embodiments, the anti-microbial filter 608 is a mechanical filter coated with antimicrobial compounds or materials as are known in the art.

In some embodiments, as described in further detail below, the destructive filtration process includes using an ultraviolet destruction mechanism ranging from 200 nm to 260 nm; PCO or PECO destruction components and processes; or negative ion, or bipolar, or plasma ion antimicrobial destruction systems and processes. In embodiments, the system synergistically combines the effects of photo-electrochemical oxidation (PECO) or photochemical oxidation (PCO) with ionization (unipolar, negative-ion, bipolar or cold plasma) to treat (i.e. disinfect and/or sterilize) both the air inside and outside of the system. The system of the present specification may also employ an activated charcoal or carbon filter to remove harmful ions, ozone, or volatile organic compounds (VOCs). In some embodiments, the ultraviolet destruction mechanism includes far-UV. In some embodiments, the ultraviolet destruction mechanism includes UV-C. In some embodiments, an ionizer, is deployed within at least one of the filter chambers or the UV-C chamber to aid with sanitization, VOC removal, and particulate filtration. In embodiments, the ionizer produces between 1 million and 1 billion ions/cc of air and generate only negative or both negative and positive ions.

At step 664, the filtered air is delivered to a personal air delivery device (or personal facial, head, or neck appliance) 612, where at least a first portion of the filtered air 610 is breathed into a user's lungs at step 666. Concurrently, at least a second portion of the filtered air 610 is circulated away from the person at step 668, via both the personal air delivery device 612 and the user's exhaled breath. As a result, at step 670, an air bubble is created around the nose and mouth of the user. The air bubble, also termed herein as an “air shield”, comprising the second portion of the filtered air 610, is created by the personal air delivery device 612 that counters the flow of infected or unfiltered air toward the person. The device 612 also pushes any airborne pathogens away from the user, and consequently dilutes or prevents the pathogens from reaching the persons airways and infecting the person. The volume of the “air shield” created around a user's nose and face, is, in some embodiments, greater than 1 L. The volume of the air shield is adequate to meet 50% or more of a user's peak flow requirement during inhalation.

In some embodiments, the second portion of filtered air (that which is circulated away from the person) is greater than the first portion of filtered air (that which is breathed into the person's lungs). In some embodiments, the personal air management device of the present specification is used in a physical state of the user, where the physical state ranges from sedentary to active. A user may wear the device while sitting, walking, and/or running, to achieve the benefits of inhaling clean, filtered air.

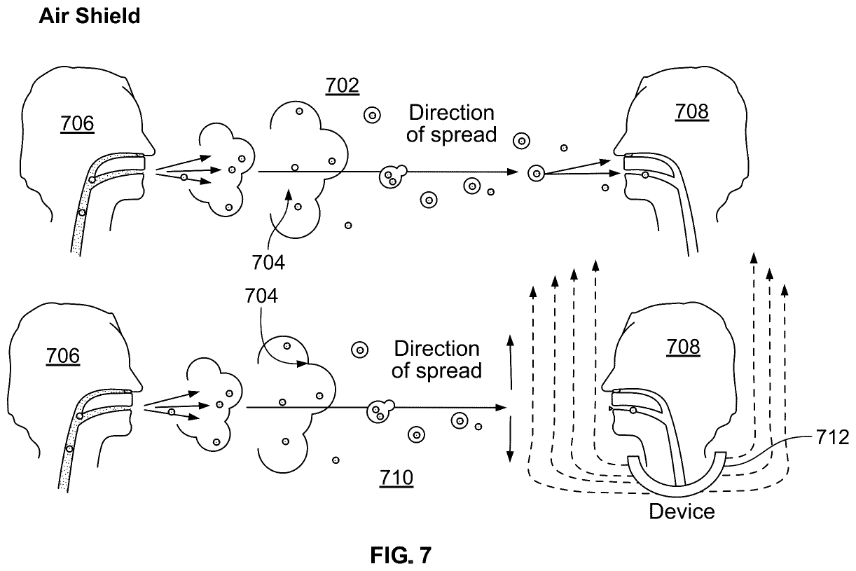

In embodiments of the present specification, the personal air management system is a wearable device that creates an air shield around a person as described with respect to FIG. 5 above. FIG. 7 is a schematic diagram showing air flow with and without use of a personal air management system in accordance with various embodiments of the present specification. A first illustration 702 shows flow of air 704 exhaled by a first person 706. Air 704 may be contaminated with bacteria, viruses, aerosolized microbes, or any other infectious constituents. Air 704 may be inhaled directly by a second person 708 in proximity of the first person 706. A second illustration 710 shows flow of air 704 exhaled by first person 706 is diverted in different directions, away from the second person 708, with the use of a personal air management system 712. In the illustrated embodiment 710, the personal air management system is implemented in the form of a neckband 712 that may be worn by person 708. Neckband 712 directs the air flow upwards, away from the face of second person 708. The re-direction of air flow creates a shield made of air (an air shield) around the second person's 708 face. In some embodiments, the neckband 712 creates the air shield by blowing filtered air outwards from the device of neckband 712. The outward flow of filtered air from neckband 712 impedes or blocks the flow of contaminated air from first person 706, thereby minimizing or eliminating the risk of inhalation of aerosolized microbes. In addition, the flow of filtered air provides clean air for inhalation by the user 708. The flow of clean air also creates a volume of clean air around a person's nose or mouth. In embodiments, the volume of clean air space (the “bubble”) is greater than 1 L and provides for greater than 50% of the peak inspiratory flow of the user 708 during moderate to severe activity level so as the user does not inhale contaminate air from outside of the air shield clean air volume.

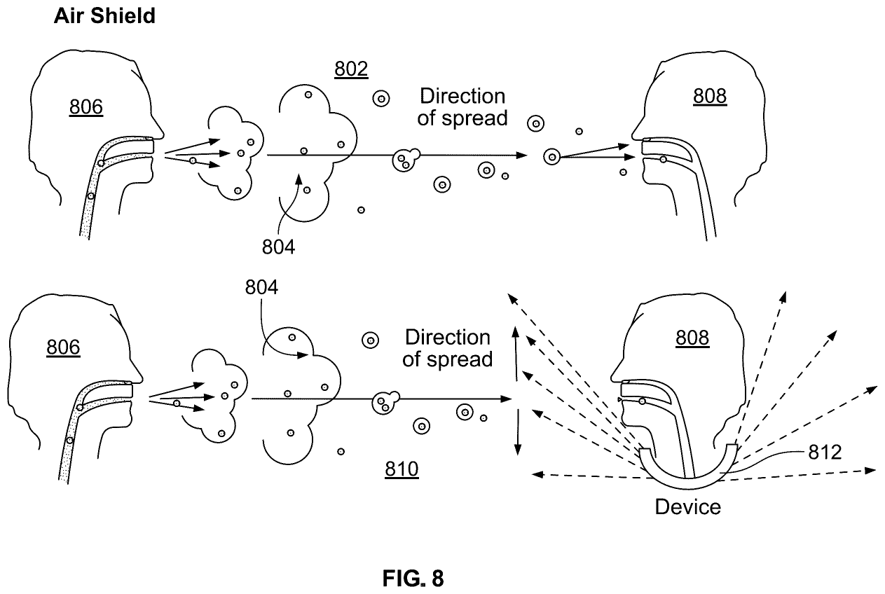

FIG. 8 is a schematic diagram showing air flow both with and without use of a personal air management system in accordance with various embodiments of the present specification. A first illustration 802 shows flow of air 804 exhaled by a first person 806. Air 804 may be contaminated with bacteria, viruses, aerosolized microbes, or any other infectious constituents. Air 804 may be inhaled directly by a second person 808 in proximity of the first person 806. A second illustration 810 shows flow of air 804 exhaled by first person 806 is diverted in different directions, away from the second person 808, with the use of a personal air management system 812. In the illustrated embodiment 810, the personal air management system is implemented in the form of a neckband 812 that may be worn by person 708. Neckband 812 directs the air flow upwards and outwards, away from the face of second person 808. The re-direction of air flow creates a shield made of air (an air shield) around the second person's 808 face. In some embodiments, the neckband 812 creates the air shield by blowing filtered air upwards and outwards from the device of neckband 812. The upward and outward flow of filtered air from neckband 812 impedes or blocks the flow of contaminated air 804 from first person 706, thereby minimizing or eliminating the risk of inhalation of aerosolized microbes.

In various embodiments, air outlet ports (for releasing air) are configured to be positioned on the neck or head band 712/812 to direct the flow of majority of the air 704/804 relatively parallel to or away from the face rather than allowing the air 704/804 to reach the person's 708/808 face, including the mouth and nose. In various embodiments. Air outlet ports are optimized to create a uniform clean air bubble around the user's nose and mouth for efficient inhalation of clean purified air.

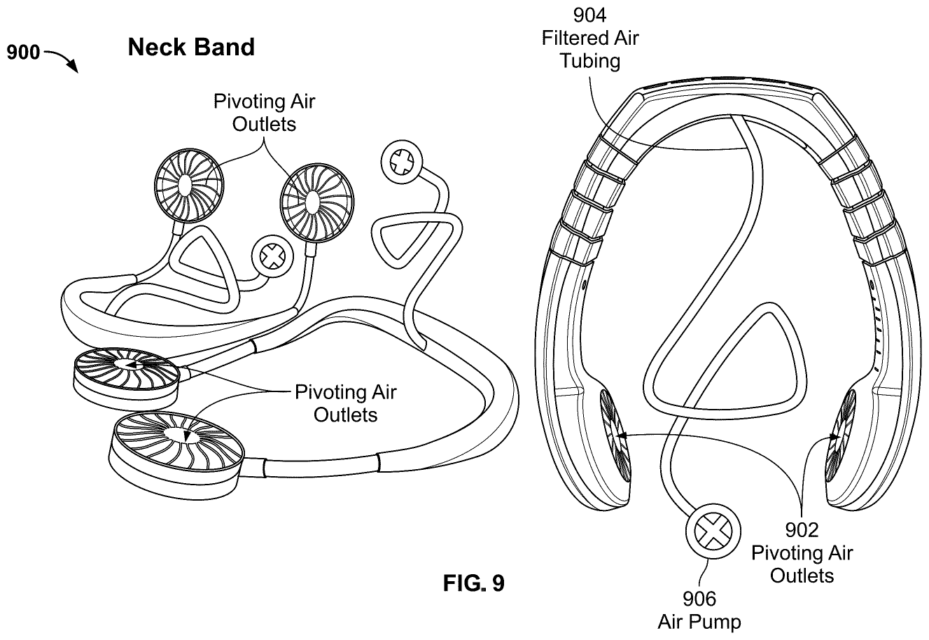

FIG. 9 is an illustration of an exemplary neck band that is designed for use with embodiments of the present specification. Neck band 900 includes pivoting air outlets or fans 902 integrated therein to create an “air shield” around the user's nose and mouth. Neck band 900 is connected to an air pump 906 via a tubing 904. Tubing 904 is used to deliver filtered air from the air pump 906 to the neck band 900. Pivoting fans 902 can be adjusted by the user to direct the airflow in a direction which is both comfortable and optimized for maximizing efficacy of the air shield. In some embodiments, the fans 902 are parallel to face of the user and may be adjusted in a range of up to 45 degrees in an outward direction.

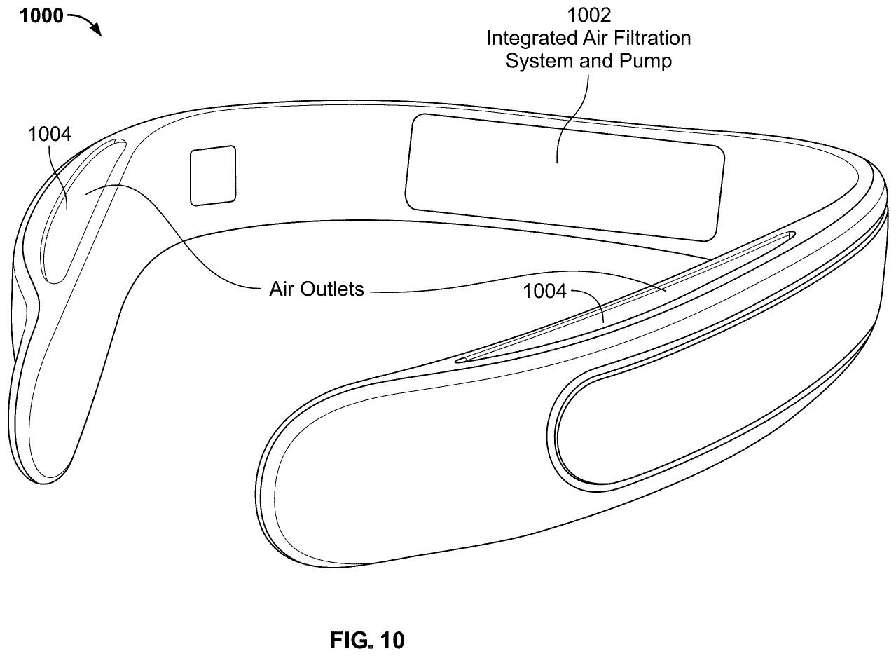

FIG. 10 is an illustration of a neck band that includes an integrated air filtration system and pump 1002 housed within the neckband 1000. Neckband 1000 further includes air outlets 1004 for creating an air shield around the user. In this embodiment, filtered air delivery tubing is internal to the neck band 1000 and is used to connect the integrated air filtration system and pump to the air outlets 1002 internally.

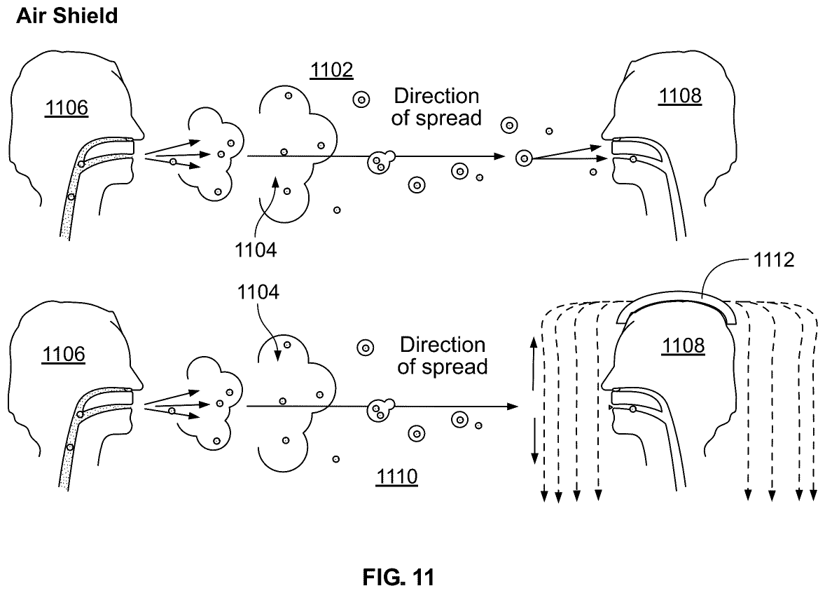

FIG. 11 is a schematic diagram showing air flow both with and without the use of a personal air management system in accordance with various embodiments of the present specification. A first illustration 1102 shows flow of air 1104 exhaled by a first person 1106. Air 1104 may be contaminated with bacteria, viruses, aerosolized microbes, or any other infectious constituents. Air 1104 may be inhaled directly by a second person 1108 in proximity of the first person 1106. A second illustration 1110 shows flow of air 1104 exhaled by first person 1106 is diverted in different directions, away from the second person 1108, with the use of a personal air management system 1112. In the illustrated embodiment 1110, the personal air management system is implemented in the form of a headband 1112 that may be worn by person 1108. Headband 1112 directs the air flow downward, away from the face of second person 1108. The re-direction of air flow creates a shield made of air (an air shield) around the second person's 1108 face. In some embodiments, the headband 1112 creates the air shield by blowing filtered air outwards from the device of headband 1112. The outward flow of filtered air from headband 1112 impedes or blocks the flow of contaminated air from first person 1106, thereby minimizing or eliminating the risk of inhalation of aerosolized microbes. In addition, the flow of filtered air provides clean air for inhalation by the user 1108. The flow of clean air also creates a volume of clean air around a person's nose or mouth. In embodiments, the volume of clean air space (the “bubble”) is greater than 1 L and provides for greater than 50% of the peak inspiratory flow of the user 1108 during moderate to severe activity level so as the user does not inhale contaminate air from outside of the air shield clean air volume.

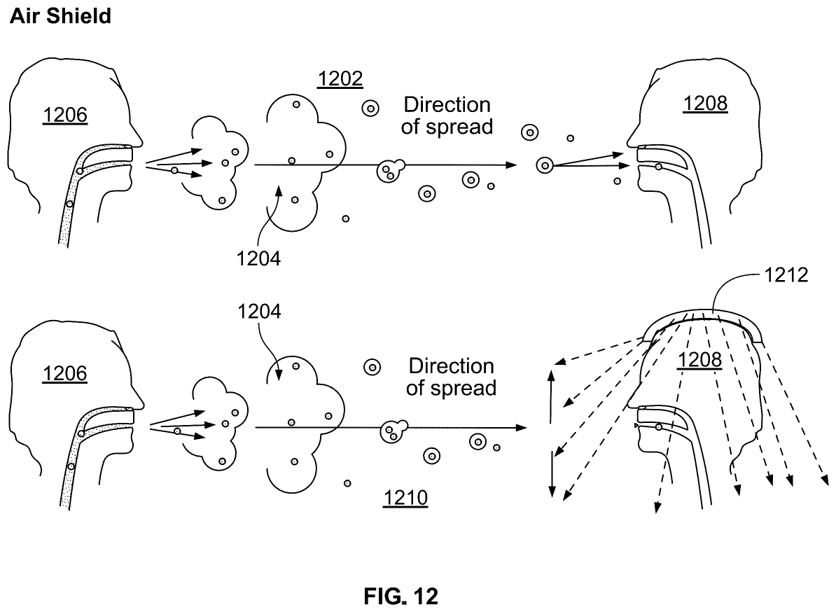

FIG. 12 is a schematic diagram showing air flow both with and without use of a personal air management system in accordance with various embodiments of the present specification. A first illustration 1202 shows flow of air 1204 exhaled by a first person 1206. Air 1204 may be contaminated with bacteria, viruses, aerosolized microbes, or any other infectious constituents. Air 1204 may be inhaled directly by a second person 1208 in proximity of the first person 1206. A second illustration 1210 shows flow of air 1204 exhaled by first person 1206 is diverted in different directions, away from the second person 1208, with the use of a personal air management system 1212. In the illustrated embodiment 1210, the personal air management system is implemented in the form of a headband 1212 that may be worn by person 1108. Headband 1212 directs the air flow downward and outward, away from the face of second person 1208. The re-direction of air flow creates a shield made of air (an air shield) around the second person's 1208 face. In some embodiments, the headband 1212 creates the air shield by blowing filtered air outwards from the device of headband 1212. The outward flow of filtered air from headband 1212 impedes or blocks the flow of contaminated air from first person 1206, thereby minimizing or eliminating the risk of inhalation of aerosolized microbes. In addition, the flow of filtered air provides clean air for inhalation by the user 1208. The flow of clean air also creates a volume of clean air around a person's nose or mouth. In embodiments, the volume of clean air space (the “bubble”) is greater than 1 L and provides for greater than 50% of the peak inspiratory flow of the user 1208 during moderate to severe activity level so as the user does not inhale contaminate air from outside of the air shield clean air volume.

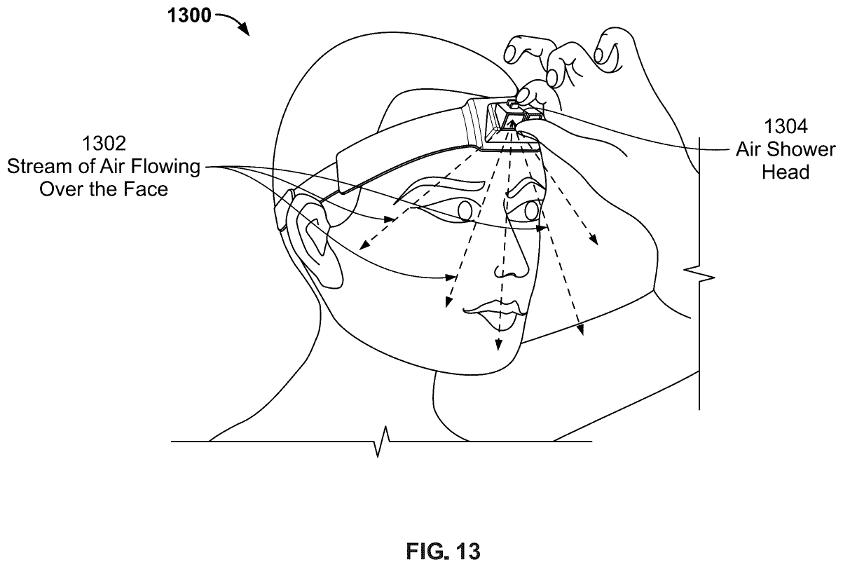

FIG. 13 is an illustration showing a headband 1300 that creates an air shield around the user's head where the air flow 1302 is directed downwards in a “shower” flow pattern. Headband 1300 creates a flow of air over the face produced by a plurality of openings in the headband. The openings are housed in a structure 1304 that can be tilted to direct the flow of air 1302 over the face to create an air shield balancing the user's comfort with efficacy of the air shield.

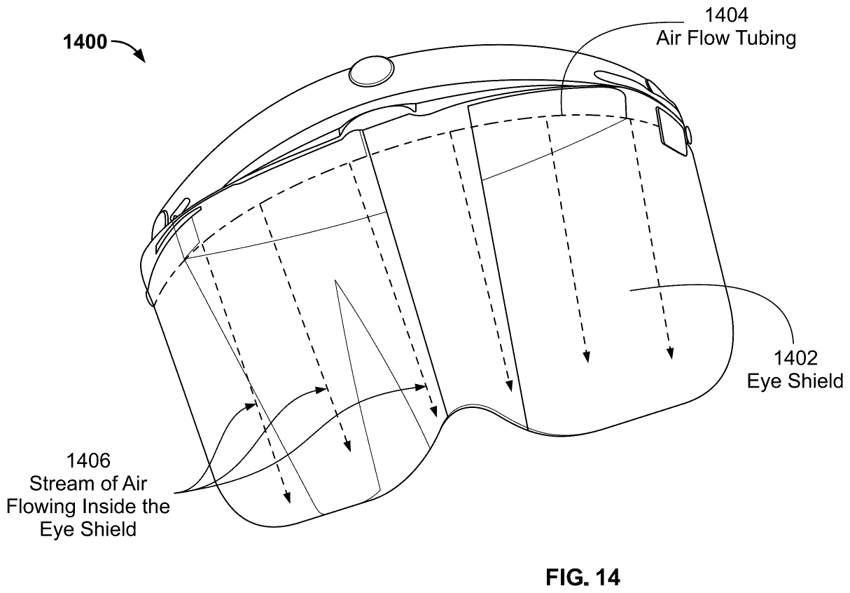

FIG. 14 is an illustration showing an eye shield or face shield 1400 that may be used with embodiments of the present specification to provide enhanced protection in combination with an air shield, around the face and head of a user. Eye shield 1400 includes a pair of goggles 1402 with an air management system integrated therein wherein the air management system includes an air flow tubing 1404 integrated on a top portion of the eye shield to direct a stream of air 1406 to flow within the eye shield creating a clean air shield protecting the patient's nose, mouth and face from aerosolized pathogens.

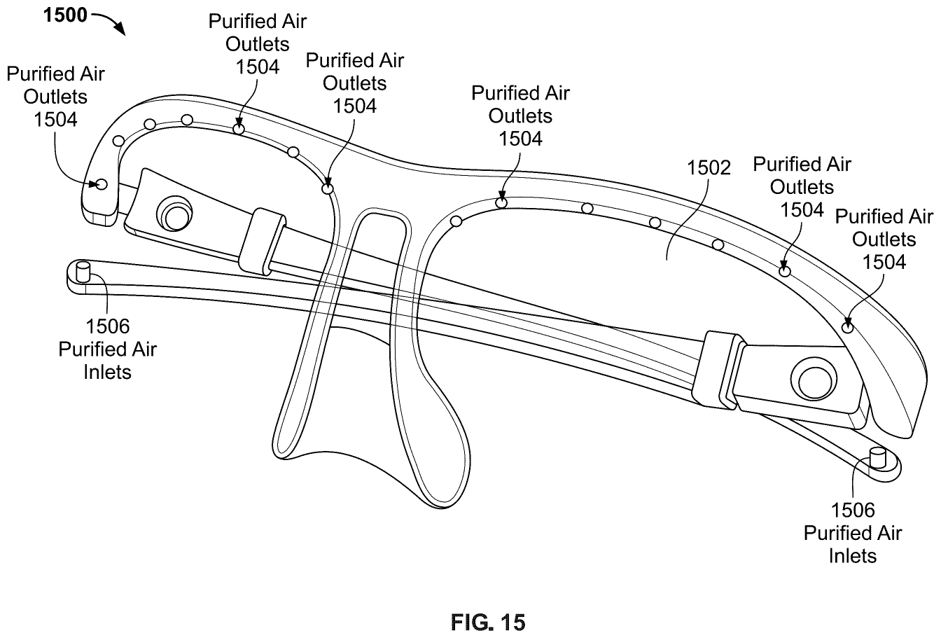

FIG. 15 is an illustration showing another configuration of an eye shield or face shield 1500 that may be used with embodiments of the present specification to create an air shield around the face and head of a user. Eye shield 1500 includes a pair of goggles or eye frame 1502 with an air management system integrated therein wherein the air management system includes a plurality of purified air outlets 1504 integrated on a top portion of the eye shield to direct a stream of air to flow within the eye shield creating a clean air shield. In addition, the eye frame 1502 includes at least two air purified air inlets 1506 for receiving purified air.

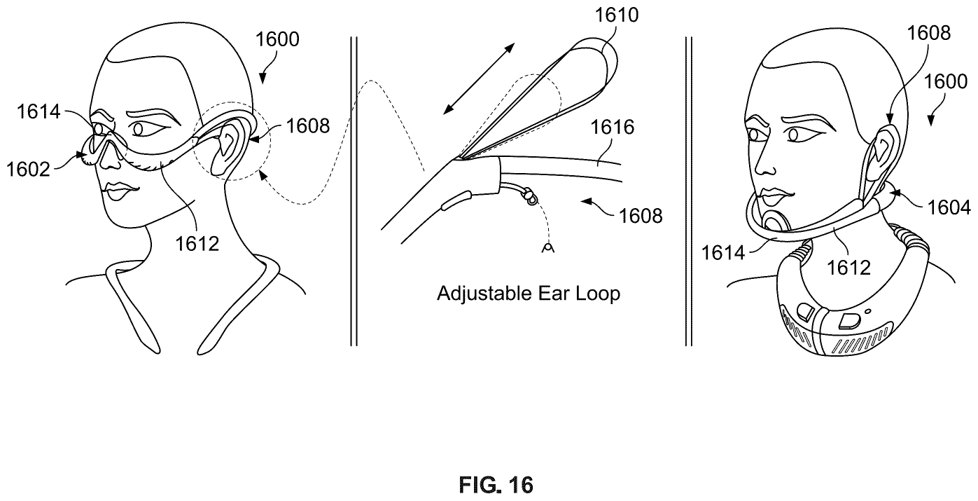

FIG. 16 illustrates an embodiment of a personal air filtration system (PAFS) 1600 that may be used in conjunction with the various embodiments of the air management system shown in FIGS. 7 to 10, in accordance with some embodiments of the present specification. In various embodiments, the PAFS is worn proximate a person's nose and mouth to create a clean air delivery to that region. The opening or ports on PAFS are optimized a clean air bubble around the wearer's nose and mouth which is used by the wearer to breath. The clean air flow also pushes the external pathogens or contaminants away from the wearer's nose and mouth thus reducing the risk of inhalation of such pathogens or contaminants. In embodiments, a PAFS 1600 is wearable by a user on the bridge of the nose, as shown in an illustration 1602, or alternatively below the chin as shown in an illustration 1604. The PAFS 1600 includes an adjustable ear loop 1608 on each side that enables the user to fit the system 1600 above or around the ears on both sides of the system 1600. The adjustable ear loop 1608, on either side, includes a flexible band 1610 that may be worn by the user around the ear. The band 1610 is attached to an arm 1612 on a first side of the system 1600 that extends in a shape similar to the temples of a pair of eyeglasses. A similar configuration is provided on a second side of system 1600 to fit the other side of the face and ear of the user. The two arms 1612 meet in front of the user's face where one or more air outlets 1614 are provided. In some embodiments, PAFS 1600 includes first set of components that are reusable, and a second set of components that are disposable or meant for single use. The reusability of the first set of components may depend on the use scenario and requirement. In embodiments, the bands 1610 and the arms 1612 are reusable, and the air outlets 1614 are meant for single use. The rear portions of the arms 1612 are fitted with at least one tube 1616 that connects the system 1600 to an air filter, which is also reusable. In embodiments, filtered air is blown from an air filtration system and pump, towards air outlets 1614 through tubes 1616 and arms 1612. In embodiments, the air filtration system and pump connected to the system 1600 is also operated for the neck wearables systems such as those described in context of FIGS. 7, 8, 9, and 10.

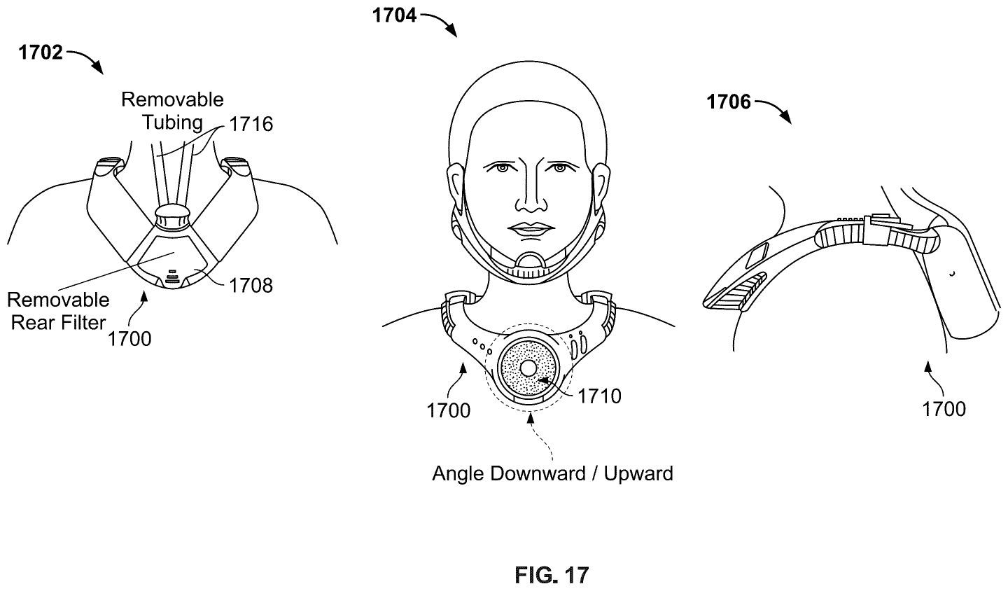

FIG. 17 illustrates embodiments of a neck wearable PAFS 1700 in accordance with some embodiments of the present specification. A representation 1702 illustrates a rear elevation view, a representation 1704 illustrates a front elevation view, and a representation 1706 illustrates a side elevation view of the system 1700. In an embodiment, the system 1700 is worn by a user by placing it around the neck of the user so that the sides of the system 1700 rest on the shoulders of the user. Therefore, the representation 1704 appears in front of the neck of the user. An air outlet 1710 at the center of the front side of system 1700 provides filtered air for the user. In some embodiments, a direction of the air outlet 1710 is adjustable. In some embodiments, a direction of the air outlet 1710 is adjusted in an upward or downward direction manually or electrically by the user. In some embodiments, a direction of the air outlet 1710 is adjusted in an upward or downward angle manually or electrically by the user. In some embodiments, an angle of adjustment varies up to 15 degrees in either the upward or the downward direction. A stop mechanism is configured in system 1700 to prevent movement of the air outlet beyond a specified angle. Representation 1702 illustrates tubes 1716 that are configured to connect an air filtration system and pump 1708 to either side of a PAFS system that is wearable on a user's face such as those shown in FIG. 16. In embodiments, the tubes 1716 are removable, and enable the user to separate the face and neck wearable systems.

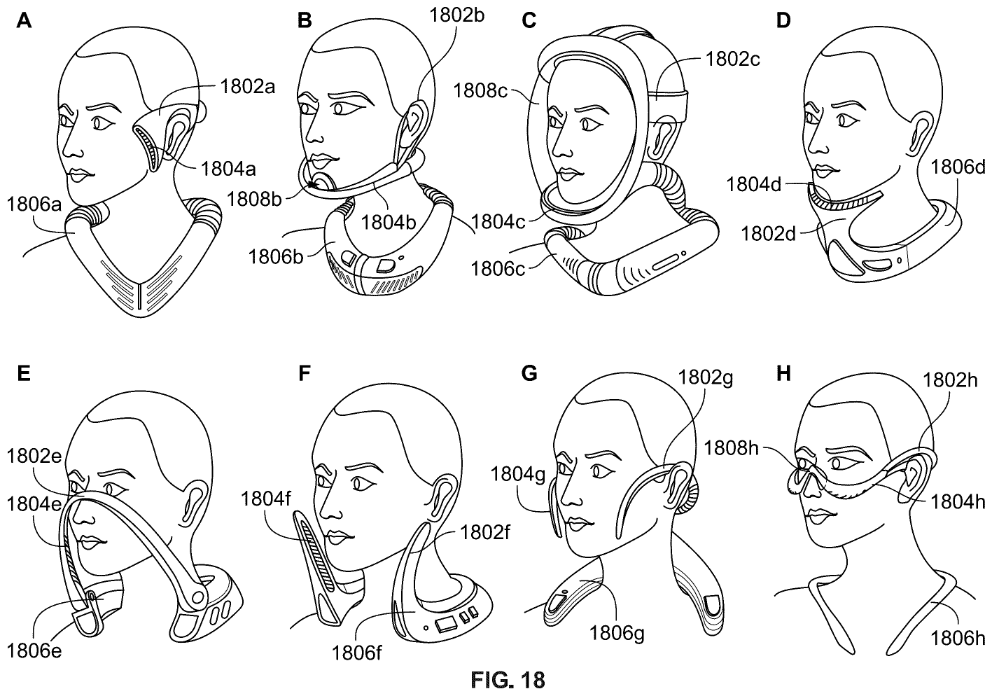

FIG. 18 illustrates multiple embodiments of PAFS, in accordance with the present specification. The embodiments shown include at least a first portion that is worn by the user in close proximity to or on the face of the user and at least a second portion that is worn around the neck of the user. In some embodiments, the first portion may be for single use, while the second portion is configured to be reusable. In further embodiments, some parts of the first portion are reusable, whereas other parts that are in direct contact or in proximity of the nose and/or mouth of a user, is configured for single use. In the embodiments, an air filtration system and pump are integrated into at least one of the first and second wearable portions, which provides filtered air through air outlets for the user's intake.

Referring to FIG. 18, an embodiment A illustrates a headband 1802a that is configured to rest above the ears of the user. Air outlets 1804a are provided in the front open sides of the headband 1802a. The headband 1802a is connected at the backside of the user via a tubing (not shown), with an air filtration system and pump that may be integral to another wearable portion 1806a of the PAFS. The portion 1806a is worn around the neck of the user, similar to a necklace, such that it can rest and balance itself on the user's shoulders. In embodiments, the neck-wearable portion 1806a also comprises one or more air outlets that manage the air around the user's face.

Embodiment B has also been described in context of FIG. 16. Referring to embodiment B, a user may wear the PAFS below the chin. The PAFS includes an adjustable ear loop 1802b on each side that enables the user to fit the system around the ears. The adjustable ear loop 1802b, is, in an embodiment, a flexible band (see FIG. 16) that may be worn by the user around the ear on each side. The band is attached to an arm 1804b on a first side of the system that extends in a cylindrical form around the face at a level below the chin. A similar configuration is provided on a second side of the system to fit the other side of the face and ear of the user. The two arms 1804b meet in front of the user's face where one or more air outlets 1808b are provided. The rear portions of the arms 1804b are fitted with at least one tube that connects the system to an air filter 1806b. In embodiments, filtered air is blown from an air filtration system and pump 1806b, towards air outlets 1808b through tubes and arms 1804b. As described with respect to Embodiment A, the air filtration and pump configuration 1806b is worn around the neck of the user, similar to a necklace, such that it can rest and balance itself on the user's shoulders. In embodiments, the neck-wearable portion 1806b also comprises one or more air outlets that manage the air around the user's face.

Embodiment C illustrates a headband 1802c that is configured to be worn as headgear around the skull of the user. Air outlets 1804c are provided in the front sides of the headband 1802c. A protective transparent shield 1808c covers the entire face of the user. The air outlets 1804c are provided within the shield 1808c in proximity to the user's face. The headband 1802a is connected with tubing's (not shown) at the back side of the user, with an air filtration system and pump that may be integral to another wearable portion 1806c of the PAFS. The portion 1806c is worn around the neck of the user, similar to a necklace, such that it can rest and balance itself on the user's shoulders. In embodiments, the neck-wearable portion 1806c also comprises one or more air outlets that manage the air around the user's face.

Embodiment D illustrates a portion 1802d that is configured to be integrated with a neck-wearable portion 1806d such that an air outlet 1804d of portion 1802d is positioned near the chin of the user. The air outlet 1804d is configured to provide an air shower in an upward direction so that the air blown out by it shields the face of the user. An air filtration system and pump may be integral to the neck-wearable portion 1806d of the PAFS. The portion 1806d is worn around the neck of the user, similar to a necklace, such that it can rest and balance itself on the user's shoulders. In embodiments, the neck-wearable portion 1806d also comprises one or more air outlets that manage the air around the user's face.

Embodiment E illustrates a portion 1802e that is configured to be integrated with a neck-wearable portion 1806e such that at least one air outlet 1804e of portion 1802e is on both sides of the user's mouth and nostrils. The air outlet 1804e is configured to provide an air shower in a direction so that the air blown out by it shields the face of the user. An air filtration system and pump may be integral to the neck-wearable portion 1806e of the PAFS. The portion 1806e is worn around the neck of the user, similar to a necklace that may be open on its front side, such that it can rest and balance itself on the user's shoulders. In embodiments, the neck-wearable portion 1806e also comprises one or more air outlets that manage the air around the user's face.

Embodiment F illustrates a portion 1802f that is configured as two elongated arms stretching upwards on either side of the user's face, from two open ends of a neck-wearable portion 1806f. At least one air outlet 1804f of portion 1802f is on both sides of the user's mouth and nostrils. The air outlet 1804f is configured to provide an air shower in a direction so that the air blown out by it shields the face of the user. An air filtration system and pump may be integral to the neck-wearable portion 1806f of the PAFS. The portion 1806f is worn around the neck of the user, similar to a necklace that may be open on its front side, such that it can rest and balance itself on the user's shoulders. In embodiments, the neck-wearable portion 1806f also comprises one or more air outlets that manage the air around the user's face.

Embodiment G illustrates a portion 1802g that is configured as headband configured to extend around the back of the user's head and brought forward to an open end with its two sides dropping vertically on the sides of the user's face. The headband is configured to rest on the user's ears, and is connected with tubing on its rear side to a neck-wearable portion 1806g. Air outlets 1804g are positioned on the two arms of portion 1802g on either side of the face, thereby creating an invisible air shield.

Embodiment H has been described in context of FIG. 16. Referring to Embodiment H, a user wears the PAFS on the bridge of the nose, as shown. The PAFS includes an adjustable ear loop 1802h on each side that enables the user to fit the system above or around the ears on both sides of the system. The adjustable ear loop 1802h, on either side, includes a flexible band that may be worn by the user around the ear. The band is attached to an arm 1804h on a first side of the system that extends in a shape similar to the temple portions of a pair of eyeglasses. A similar configuration is provided on a second side of the system to fit the other side of the face and ear of the user. The two arms 1804h meet in front of the user's face where one or more air outlets 1808h are provided. The rear portions of the arms 1804h are fitted with at least one tube that connects the system to an air filtration and pump configuration 1806h. In embodiments, filtered air is blown from air filtration system and pump configuration 1806h, towards air outlets 1808h through tubes and arms 1804h.

It may be appreciated that different configurations of neck-wearable portions may be combined with different configurations of portions that are in close-proximity to or worn around the face by a user. Additionally, the different embodiments of PAFS in accordance with the present specification may be combined with a transparent protective shield made of a light-weight material such as plastic.

FIG. 19 illustrates an embodiment of a blower that may be used with devices in accordance with the present specification. Referring to the figure, an image 1902 illustrates a top perspective view and an image 1904 illustrates a bottom perspective view of a blower 1900a. Image 1904 also illustrates electronic components 1906 used with the blower 1900a. The electronic components 1906 may include a controller configured to control the speed of the blower 1900a by adjusting the voltage output by blower 1900a. The blower 1900a may operate in a voltage range of 9-16 VDC. In embodiments, the power supplier output current is greater than or equal to the rated motor current. The motor start time may be on the order of 2 seconds, and a total weight of the controller may be approximately 135 gm. Blower 1900a includes an inlet 1908 having a diameter in a range of 5 mm to 12 mm, and an outlet 1910 with a diameter in the range of 8 to 12 mm, for passage of air. The lower the diameter, the more losses the system has (due to friction). Also, the lower the diameter, the faster the air speed leading to lower time available for purification, thereby reducing the purification efficacy. The blower 1900a may be approximately 45 mm thick. In an embodiment, blower 1900b may have an inlet diameter in a range of 7 mm to 12 mm, and preferably of approximately 20.8 mm; an outlet diameter in a range of 8 mm to 15 mm, and preferably of approximately 12 mm; and a thickness of about 37.5 mm. Filters may be included at the inlet or at the outlet or at both openings of the blower. A general expression to determine air flow that may be used is:

Air flow (expressed in flow volume per unit time, like liters per minute)=air velocity (expressed as distance per unit time)×cross sectional area (pi×radius×radius).

In some embodiments, for a tube of radius 9 mm, the air may flow from 0.64 CFM, at a velocity of 1.18 m/s and an average current of 98 mA to 10.46 CFM, at a velocity of 19.39 m/s and an average current of 748 mA. For different flow and velocity conditions, the blowers may operate at a current ranging from 101 mA to 1350 mA without a filter; and 98 to 1500 mA with a filter at the inlet.

In some embodiments, the blower is powered at an input power ranging from 1.1 W to 1.6 W. Assuming linear relationship between air flow rate and power requirement, the power required by the blower may be extrapolated by the following equation, when it creates a flowrate of 6 CFM:

$$Power = \frac{1.6 - 1.1}{0.4 - 0.1} * ( 6 - 0.1 ) + 1.1 = 11 W ( approximately )$$

In embodiments, a maximum speed of a motor for the pump may operate in a range of approximately 10.46 CFM at 8.9W and 8.41 CFM at 15.66 W.

In embodiments, an onboard rechargeable battery pack is used to power the PAFS. The battery may be selected from one of LifePO₄, Lead acid, NiCd, NiMH, LiMnₓNi_{y}CO_{z}O₂, and LiCoO₂. The type of battery may be selected based on a combination of parameters and their utility in a type of device in accordance with the present specification. The parameters may include voltage, current, capacity, weight, run time, size, and cost. Li-Ion batteries enable portability of the PAFS, and may be preferred. Li-Ion batteries have very high specific energy density (˜250 Wh/kg), have relatively low self-discharge, and require low maintenance (no memory effects). Due to very high specific energy density, they provide the maximum power possible in a given footprint. In some embodiments, a 19.4 Whr battery is provided to power the pump and other components, such as the UV-C lamp or UV-C LED, and blower, of the systems. In some embodiments, a battery of capacity 23 WHr is provided with an efficiency of about 80%. In embodiments, UV-C lamps are preferred over LEDs since lamps have about 25% efficiency, whereas UV-C LEDs have about 2% efficiency.

In embodiments, the number of UV lamps (or LEDs) used for disinfecting the air depends on the rate of air flow, which may be based further on the user's breathing. In some embodiments using UV-C lamp, the number of lamps may range from 5 to 35, and preferably from 7 to 32. In some embodiments, the dimensions of each UV lamp may be approximately 5 mm by 127 mm. The weight of each lamp may vary for different types of devices, and may range from 0.9 to 9 lbs. The battery power required to operate the UV lamps may range from 19 to 93 WHr, with a battery life (before recharge) in a range of 0.9 to 9 hours. In an embodiment, the battery power input requirement for a UV lamp with 25% efficiency is 9.6 W. In an embodiment, a UV-C lamp is low voltage (ranging from 6V to 24V). The length of the lamp is in a range of 50 mm to 115 mm, and a width in a range of 5 to 15 mm. In embodiments, the UV-C lamp operates on the order of greater than 25 μW/cm², and ideally, greater than 100 μW/cm² (220 nm to 280 nm wavelength). The temperature of the lamp ranges from 25 degrees Celsius to 60 degrees Celsius. In embodiments, the exposure time of air to the lamp is greater than or equal to 1 second for optimal efficacy.

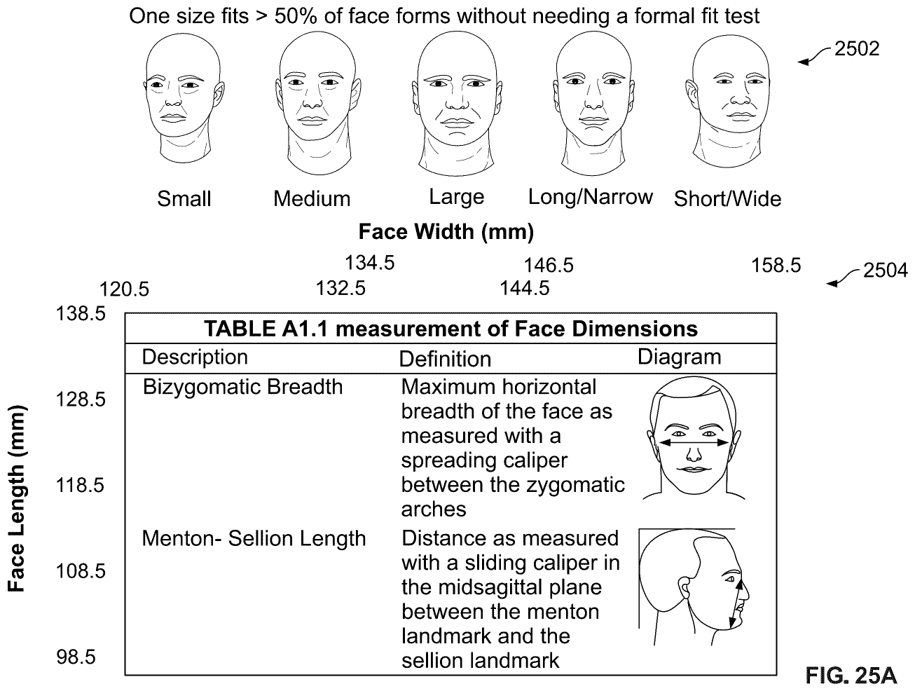

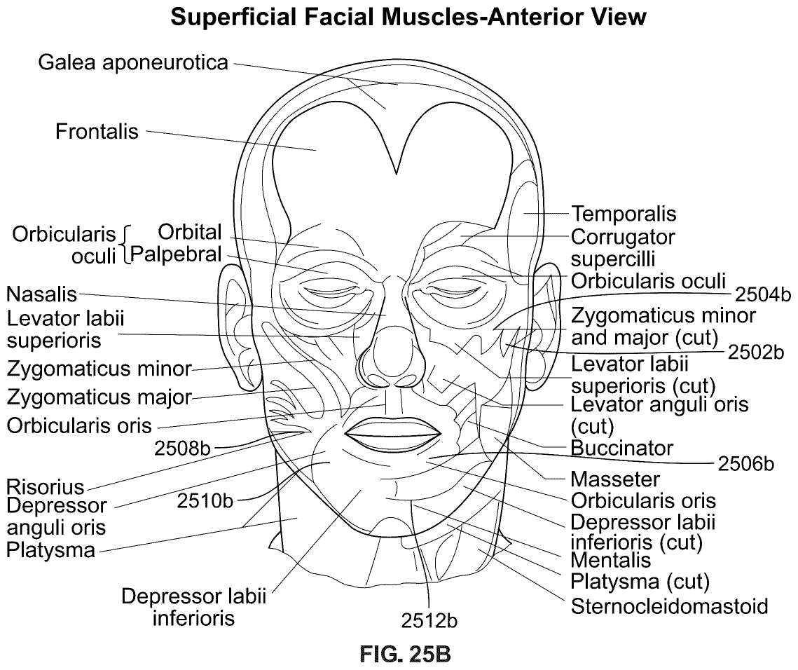

A coil or tube, which is fabricated from Ti doped clear quartz, Vycor, or high silica glass is coiled around UV-C lamp. In some embodiments, a height of the UV-C lamp with the coil wound around it is on the order of 24 mm. The coil or tube has an inner diameter ranging from 3-4 mm, and is preferably on the order of 4 mm. The diameter of the full coil or tube is on the order of 6 mm. In embodiments, the coil or tube has a wall thickness on the order of 1 mm. It should be understood by those of ordinary skill in the art that any diameter or material of tubing may be used with the embodiments described herein as long as it achieves the necessary operational parameters. The coil or tube further includes an air inlet for receiving ambient, and possibly contaminated air, and an air outlet for outputting filtered, clean air. The use of a coil increases the path of air flow in a small space, thus increasing UV exposure time. Embodiments of the coil or tube will be subsequently described in context of FIGS. 25A to 25D as well, and is referenced herein for details.

In embodiments, devices of the present specification have ducts or chambers in the form of cartridges, to carry clean air. The UV lamps are incorporated along a central axis of the spiral tube/coil (see FIGS. 25B and 25C) that comprises the ducts or chambers. An interior of the duct or chamber is lined with highly reflective media that may provide up to five times amplification of UV flux density to achieve the required disinfection (0.004 J/cm²). The thickness of such media may range from 0.2 mm (approximately 80% reflectivity) to 2 mm (approximately 97% reflectivity). In different embodiments, the type and quantity of the application of reflective media is varied to achieve the required disinfection. In an embodiment, for a light power of 12 W for a lamp of radius 0.2 cm, a cartridge ID of 4 cm, cartridge length of 20 cm, and flow rate of 10 CFM, an inlet filter initial resistance may be of 0.09 in-H2O, an outlet filter initial resistance may be 0.2 in-H2O, and a dynamic viscosity may be of approximately 0.00001802 KG/m-s at 15° C.

The embodiments described in the present specification are configured to enable their wearers or users to turn head. The embodiments that are worn by the users over the ears allow the users to wear glasses.

In some alternative embodiments, a battery/UV pack is configured to be worn on a hip/belt or shoulders to allow more room for the other components that are worn around the neck or ears.

### CONSUMER MASKS

In embodiments, the air shields and air delivery systems including pump assembly, UV-C purified air, and other components, described in the context of FIGS. 5 to 19 may be used in combination with, or separately from, embodiments of face masks that are now described.

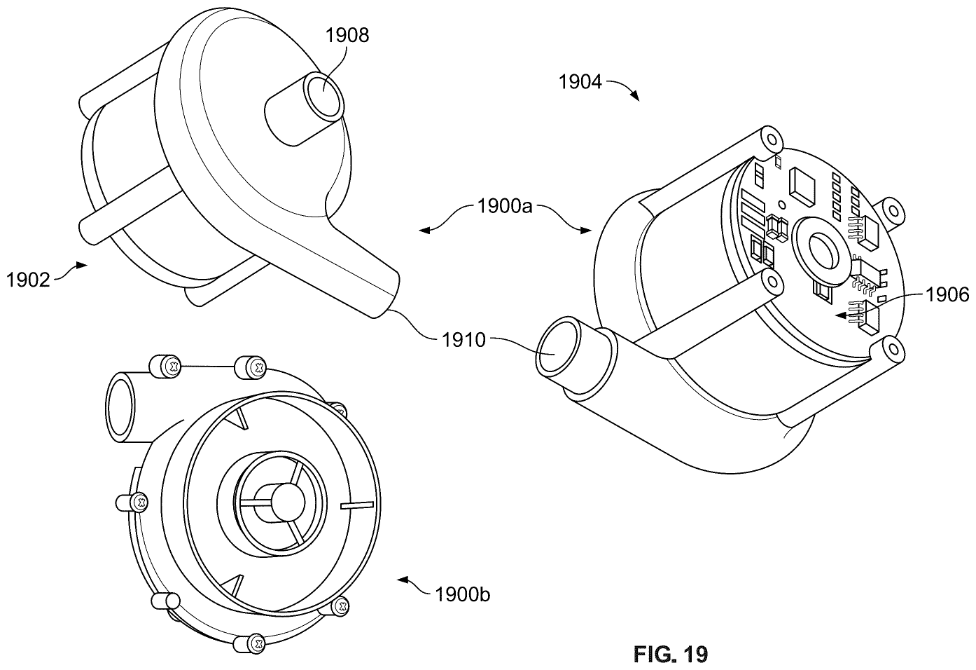

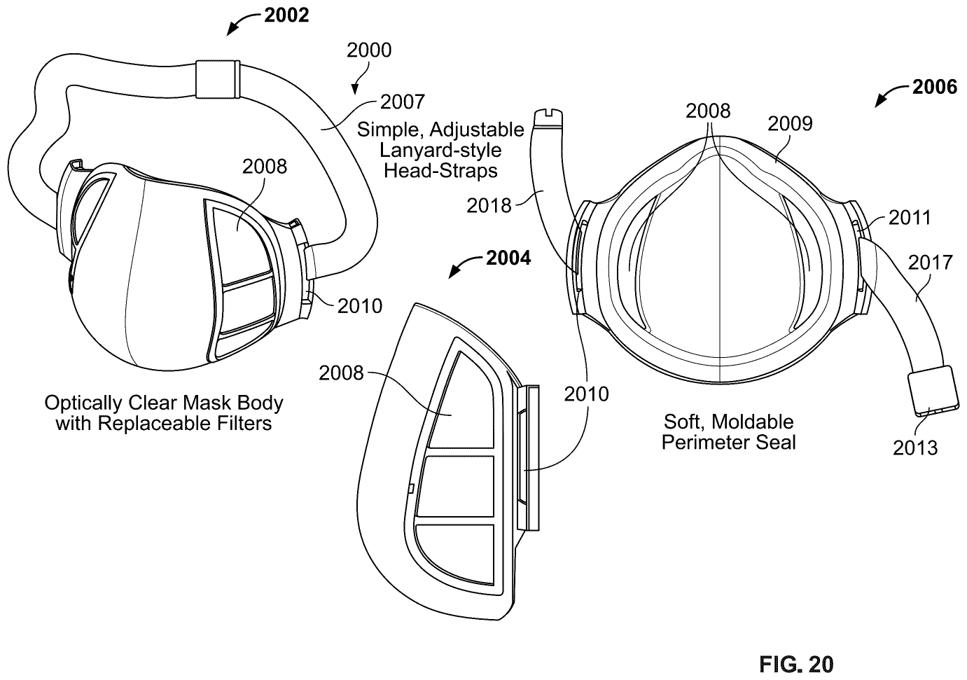

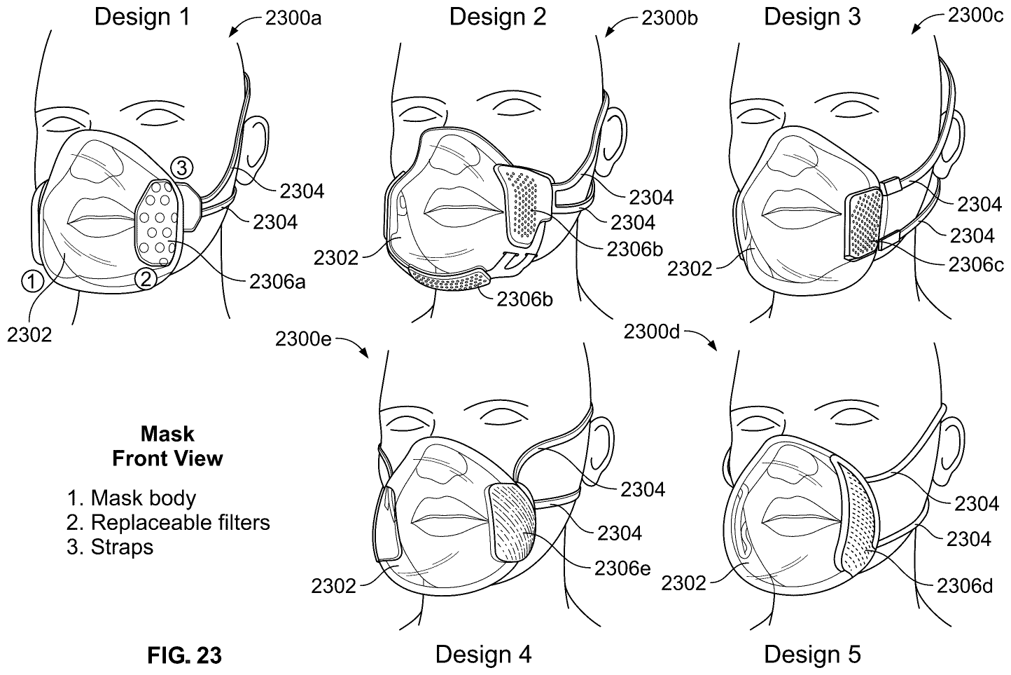

FIG. 20 illustrates a front perspective view 2002, a side view 2004, and a rear view 2006 of a mask 2000 with openings, in accordance with some embodiments of the present specification. Mask 2000 has two openings 2008—one on either side. Each opening 2008 accommodates a replaceable air filter. Mask 2000 also includes openings 2010 on either sides, at the edges of mask 2000. Opening 2010 are configured to support straps 2007 that are used by the wearer to wear and fix mask 2000 on the face. The straps are, in some embodiments, simple, adjustable Lanyard style head straps. In embodiments, the mask 2000 includes one strap 2007 extending from a first opening 2010 on a first side of the mask to a second opening 2011 on a second side of the mask. In other embodiments, the mask 2000 includes two straps 2017, 2018, a first strap 2017 extending from a first opening 2010 and a second strap 2018 extending from a second opening 2011, wherein the straps 2017, 2018 are configured to be secured together by a fastening mechanism 2013. In embodiments, the fastening mechanism comprises a clasp, latch, buckle, or tie. The straps 2007, 2017, 2018 are configured to be positioned around the back of a person's head to secure the mask in place. In some embodiments, for example, referring to FIG. 23, the mask includes more than one single strap 2007 or more than of each of the straps 2017, 2018 configured to be secured together. Referring to view 2008, a perimeter of the mask 2000 is configured with a soft moldable material that provides for a seal 2009 when the mask 2000 is worn by a user. The seal conforms with the contours on the face of the user. In various embodiments, the seal 2009 is composed of silicone, rubber, or an elastomer.

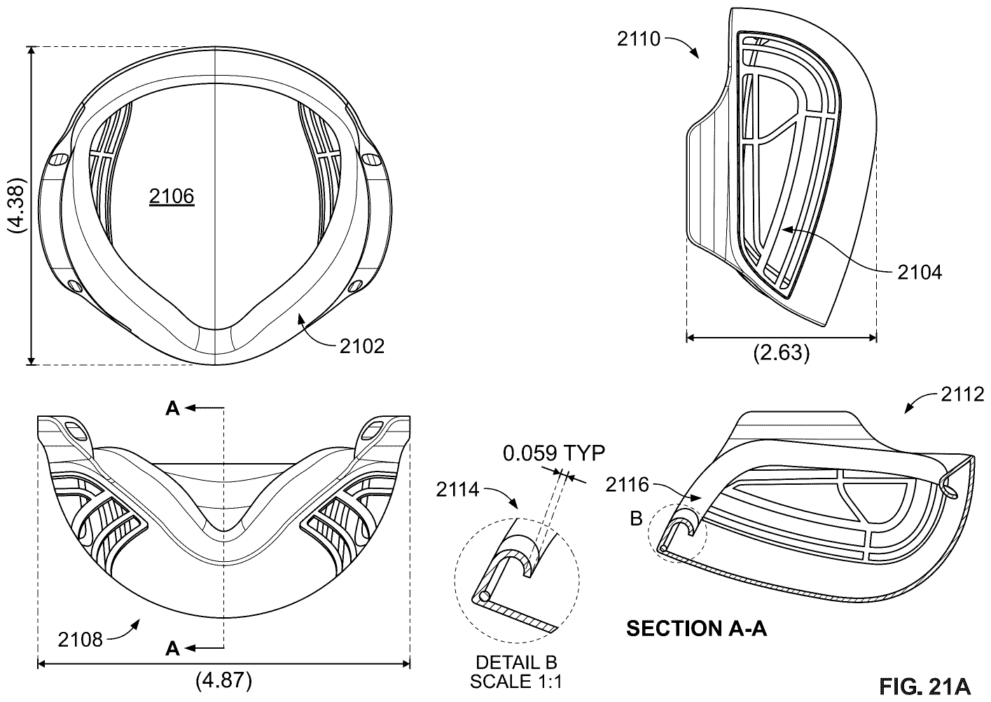

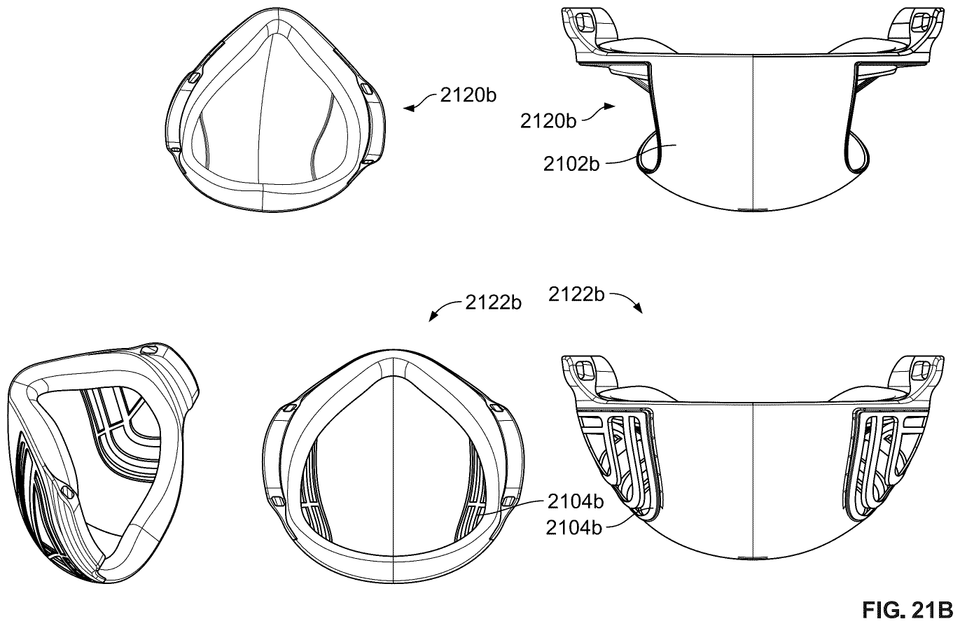

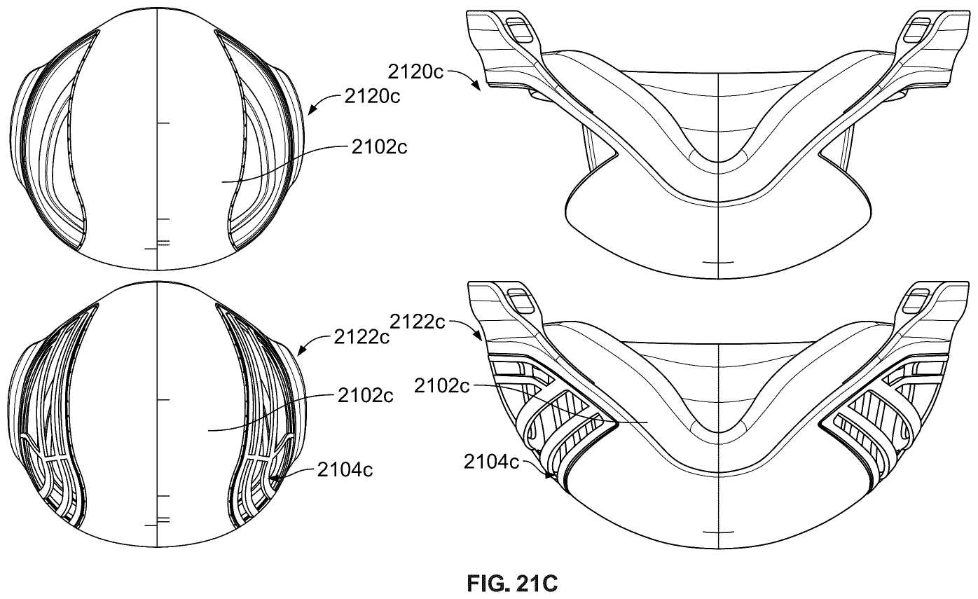

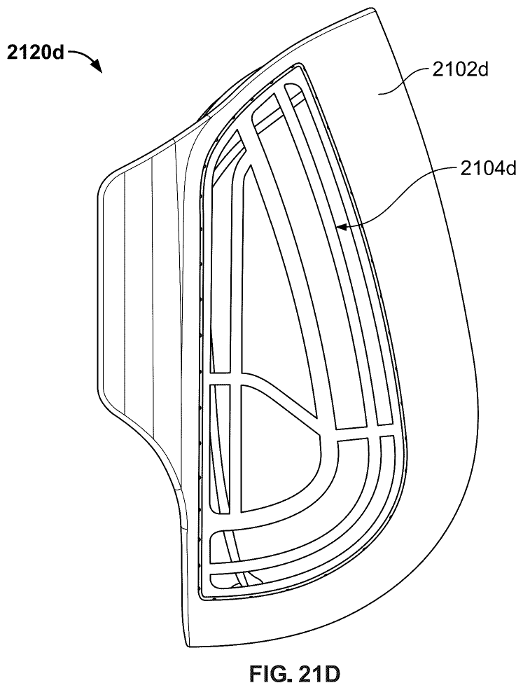

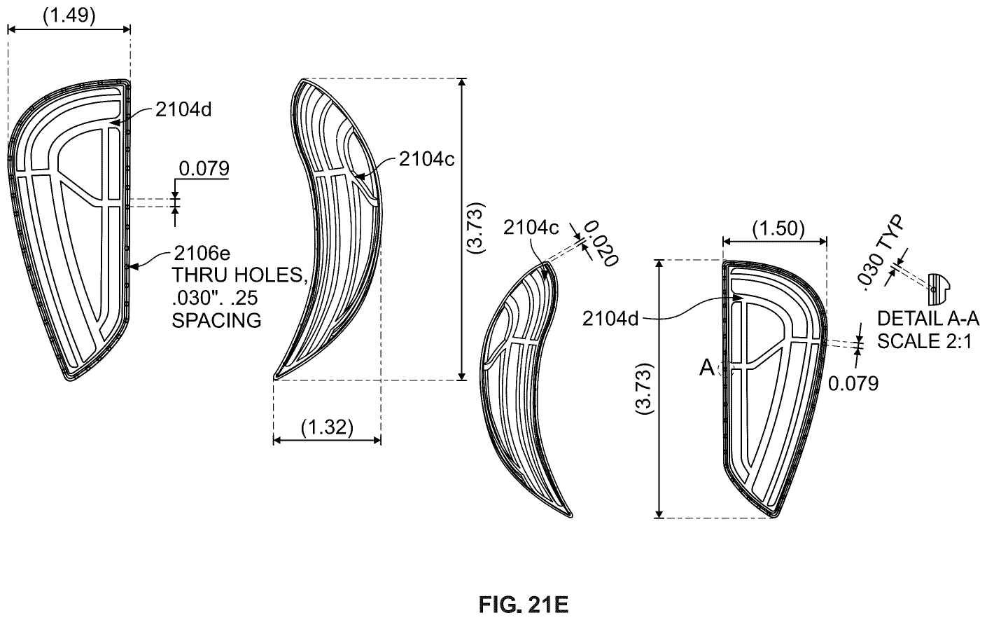

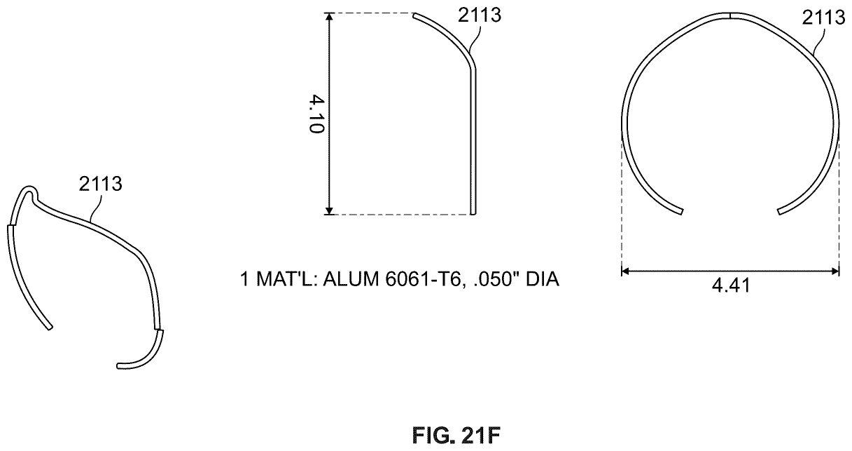

FIG. 21A illustrates different views of a silicone mask body 2102 with an insert 2104 to place a filter (not shown), in accordance with some embodiments of the present specification. In some embodiments, the insert 2104 is configured as a coarse net or a mesh within an opening/port in the shape of the filter. In other embodiments, the insert 2104 comprises a mechanical filter or destructive filter including, but not limited to, an ultra-violet (UV) filter, a photoelectrochemical oxidation (PECO) filter, a photocatalytic oxidation (PCO) filter, an ionizer, or a thermal filter. A first view 2106 illustrates a back side view of mask body 2102 the surface of which faces or comes into contact with a wearer's face. In exemplary embodiments, a vertical length of the mask body extends in a range of 2.5 to 6 inches. In some embodiments, the vertical length extends for approximately 4.38 inches. A second view 2108 illustrates a top view of the mask body 2102, showing a horizontal expanse of the body 2102 to be in a range of approximately +/−50% of 4.87 inches. A third view 2110 shows a side view of the mask body 2102, and an exemplary depth of the mask in a range of +/−50% of approximately 2.63 inches. A fourth view 2112 illustrates a cross sectional view of mask body 2102 along a central vertical cross-section. The cross-sectional view 2112 reveals a curved surface 2116 along an internal edge of body 2102, that forms a “shelf” or “ledge” or “lip”. The internal concave side of curved surface 2116 is configured to create a soft perimeter seal between the mask body and the face to prevent entry or leakage of air around the perimeter. A fifth view 2114 illustrates an enlarged sectional view of the surface 2116, which in embodiments has a thickness of approximately 0.059 inches. A wire 4 is used within through-holes in the edges of the surface 2116 to mold the mask body for a better fit. Through-holes are used to overmold a filter base into the silicone mask body. The silicone at least partially fills the through-holes to keep the filter base in place.

FIG. 21B illustrates a front and top view 2120b of a mask body 2102b with windows for inserting filters. In the same figure, back side perspective view, front view, and top view 2122b illustrate the mask body 2102b with inserts 2104b fitted within the openings/port. FIG. 21C illustrates a front and top view 2120c of a mask body 2102c with another design of openings/port for inserting filters. In the same figure, front view and top view 2122c illustrate the mask body 2102c with inserts 2104c fitted within the openings/port. FIG. 21D illustrates yet another design of an insert 2104d configured on a mask body 2102d in a side view 2120d of the mask. FIG. 21E illustrates exemplary embodiments of inserts 2104c and 2104d showing their dimensions, in accordance with some embodiments of the present specification. The figure also illustrates through-holes 2106e on a perimeter of the inserts 2104c and 2104d, which are spaced at approximately 0.25 inches from each other. In some embodiments, each through-hole 2106e has a diameter of 0.03 inches. The through-hole are configured to overmold the inserts onto the mask body. In some embodiments, the mask body is made from silicone, which at least partially fills or flows through the holes 2106e to keep the inserts in place. FIG. 21F illustrates exemplary configurations of an aluminum wire used with the inserts of the present specification. In embodiments, different malleable materials are used. The wires are used to mold the mask body around the perimeter of the mask body.

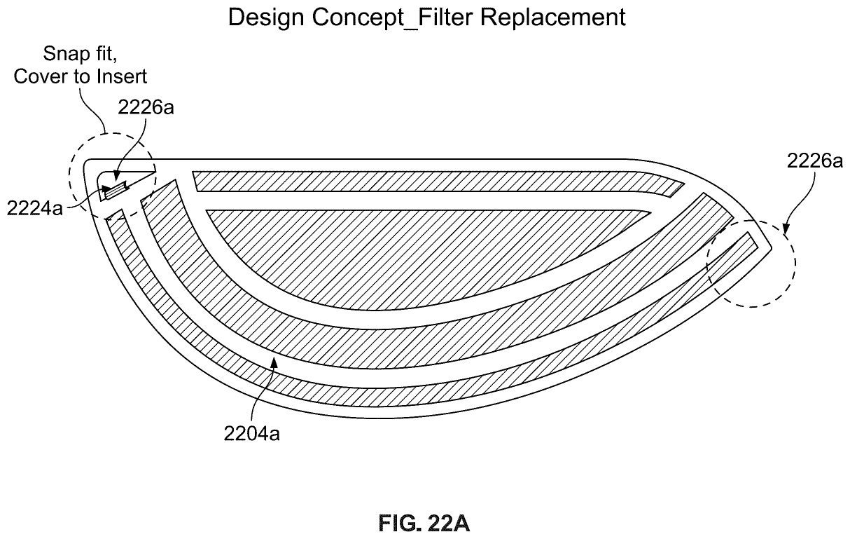

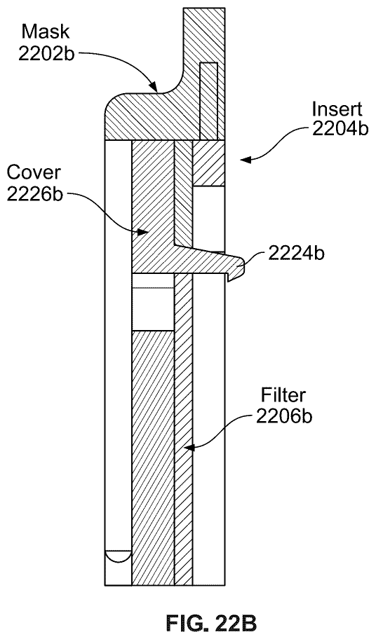

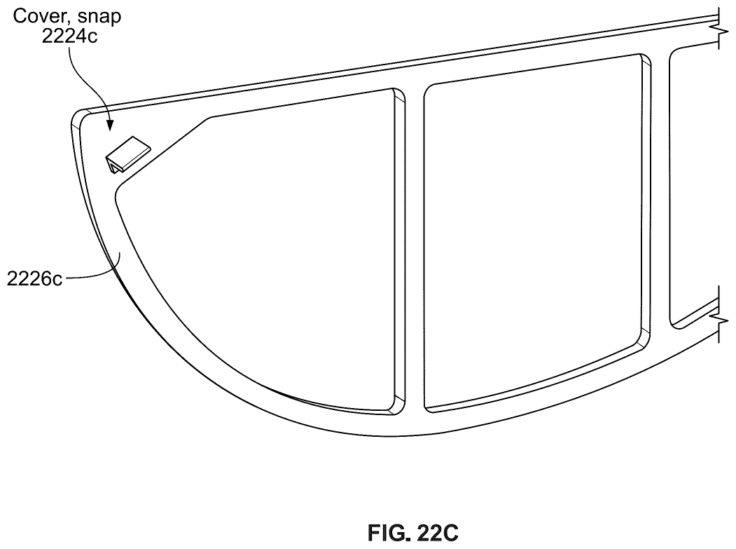

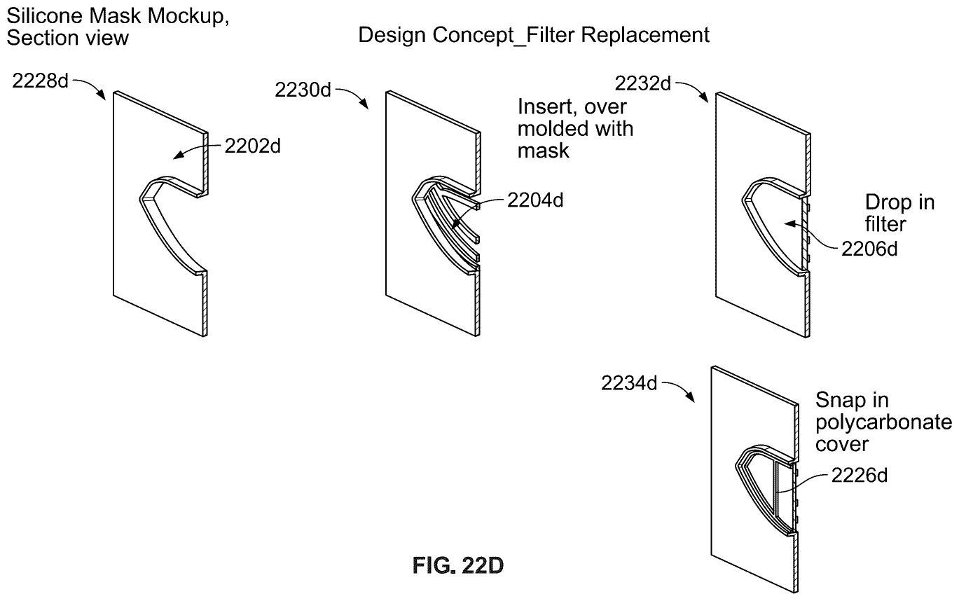

FIG. 22A illustrates an exemplary design of an insert 2204a over-molded with a snap fit attachment 2224a to cover an internal surface of a filter to support the filter that is placed between the insert 2204a and the cover 2226a (a portion of which can be seen). FIG. 22B illustrates the layers created by a cover 2226b, a filter 2206b, and an insert 2204b, in a mask body 2202b, in accordance with some embodiments of the present specification. A snap fit attachment 2224b is configured with the structure of cover 2226c in the form of a protruding hook that attaches around the insert 2204b while filter 2206b is sandwiched between cover 2226b and insert 2204b. FIG. 22C illustrates an exemplary configuration of a cover 2226c with a snap fit attachment 2224c, in accordance with some embodiments of the present specification. In embodiments, the cover 2226c is made from a polycarbonate material.

FIG. 22D is a schematic diagram illustrating a process of assembling a filter 2206d in a mock-up of a cross-section of a mask body 2202d, in accordance with some embodiments of the present specification. At step 2228d, a section view of the mask body 2202d is shown. At step 2230d, an insert 2204d is over-molded in an opening/port of the mask body 2202d. At step 2232d, filter 2206d is dropped on the insert 2204d so that the filter 2206d covers the surface of the insert 2204d. At step 2234d, a cover 2226d is snap-fitted, such as by using its snap-fit attachment (not shown), with the insert 2204d while the filter 2206d is sandwiched between the cover 2226d and the insert 2204d.

FIG. 23 illustrates five different types of mask configurations (2300a, 2300b, 2300c, 2300d, 2300e), in accordance with some embodiments of the present specification. Each configuration 2300a, 2300b, 2300c, 2300d, 2300e has a mask body 2302, straps 2304, and a unique design of filters 2306a/2306b/2306c/2306d/2306e for each configuration. In embodiments, the filters 2306a/2306b/2306c/2306d/2306e are replaceable. Configurations 2300a, 2300c, 2300d, and 2300e include filters on either sides (left and right) of a wearer's face, and configuration 2300b includes an additional filter 2306b positioned under the chin of the wearer and embedded in the mask body 2302.

In embodiments, the filters 2306a/2306b/2306c/2306d/2306e are embedded in the mask body 2302 within filter inserts. The filter inserts provide a mesh on a surface of the mask body 2302, with a space behind the mesh, to align a filter 2306 along the mesh surface, and a structure or mechanism to hold the filter 2306a/2306b/2306c/2306d/2306e in place. In some embodiments, a cover is fitted behind the filter, in an internal side of the mask, to hold the filter in its place.

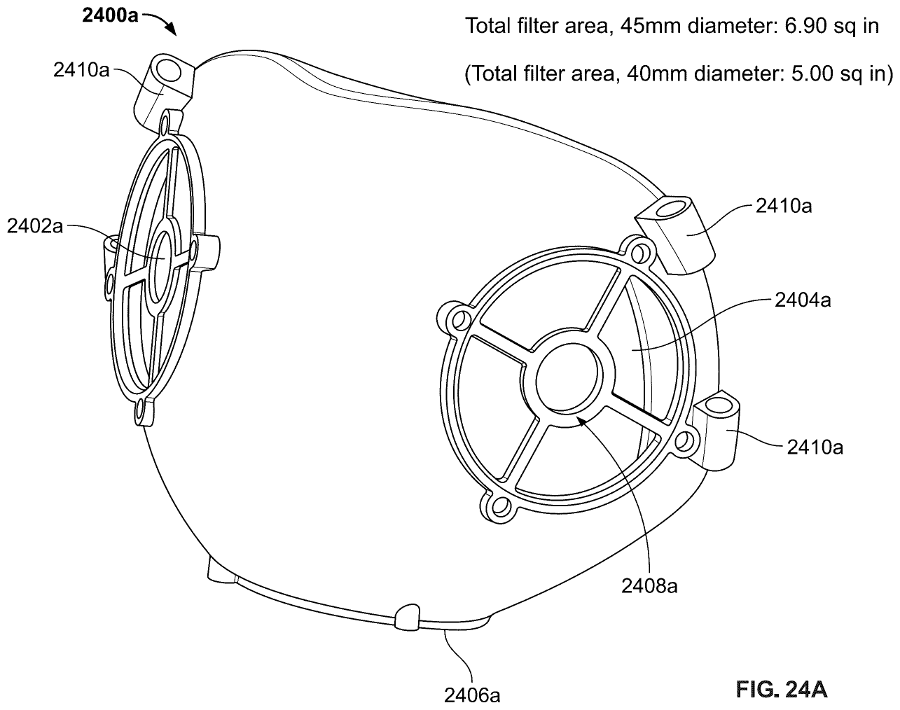

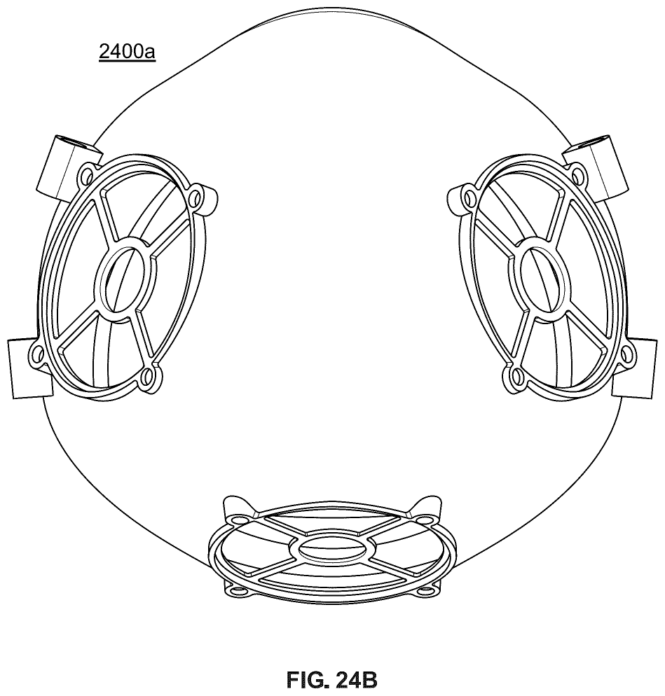

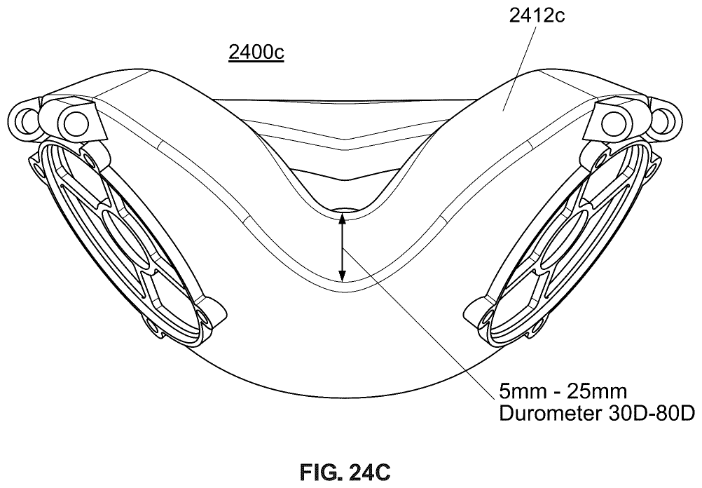

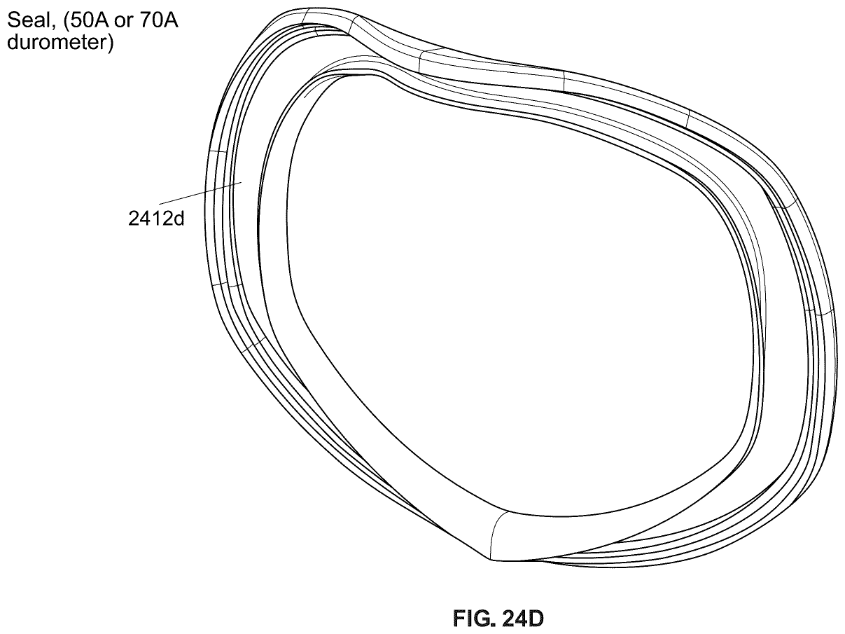

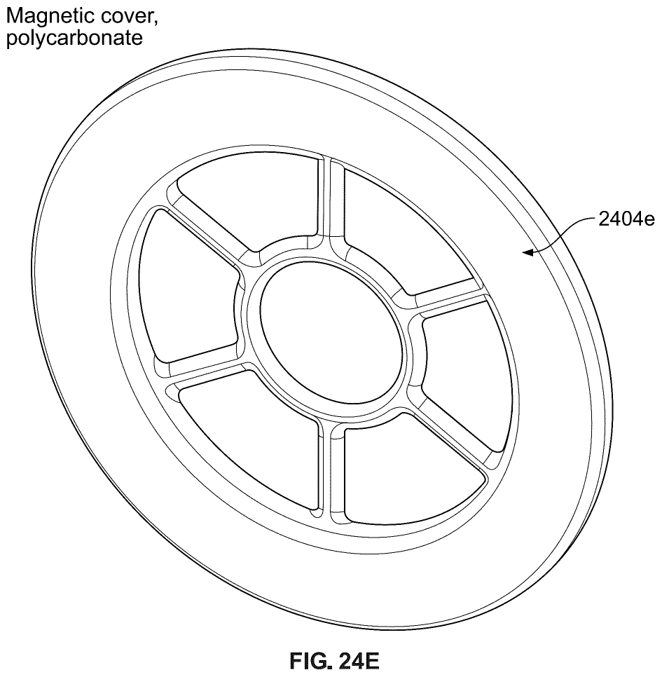

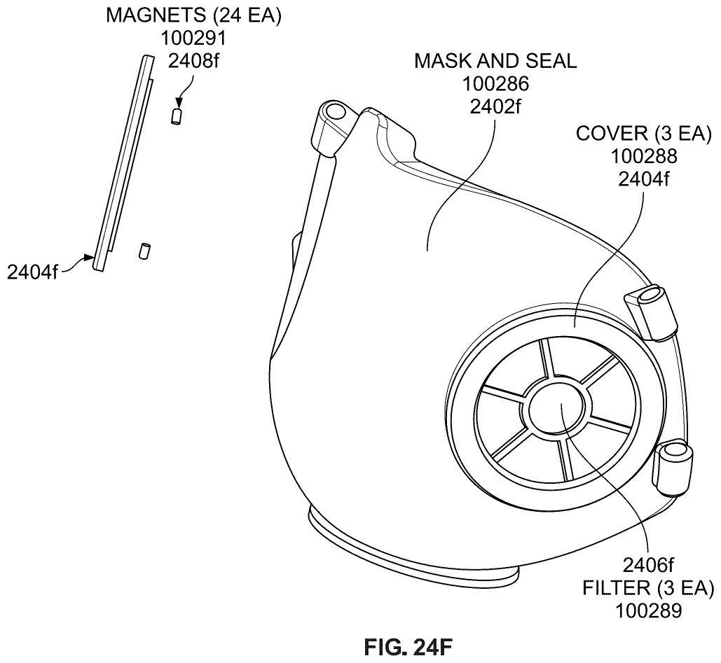

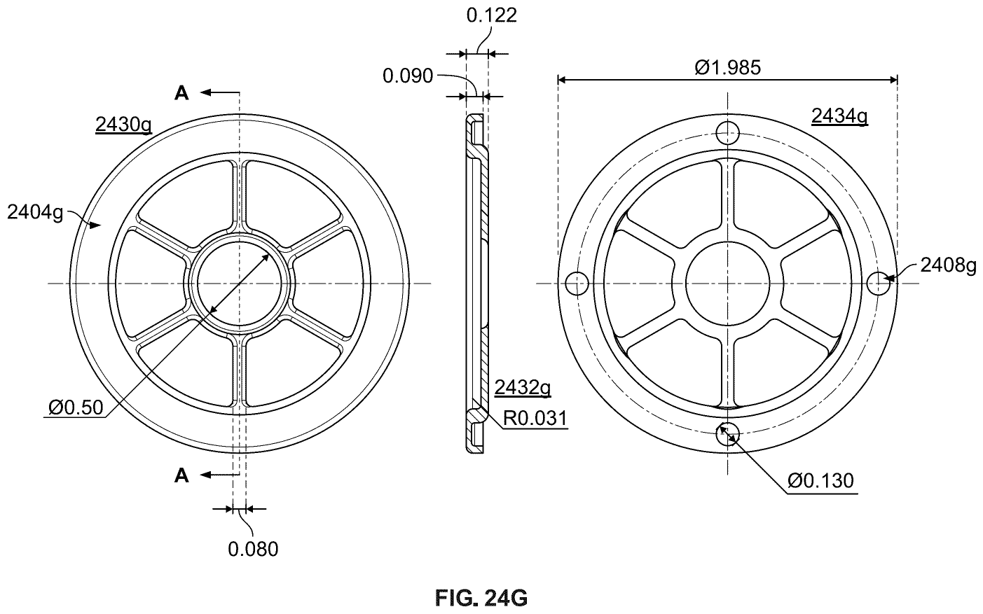

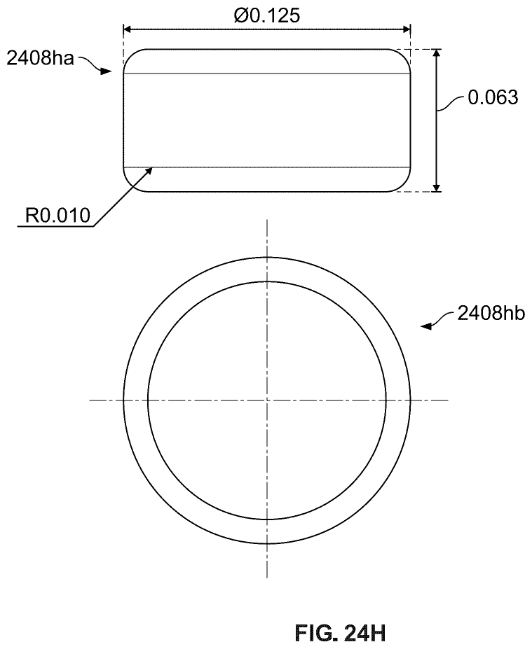

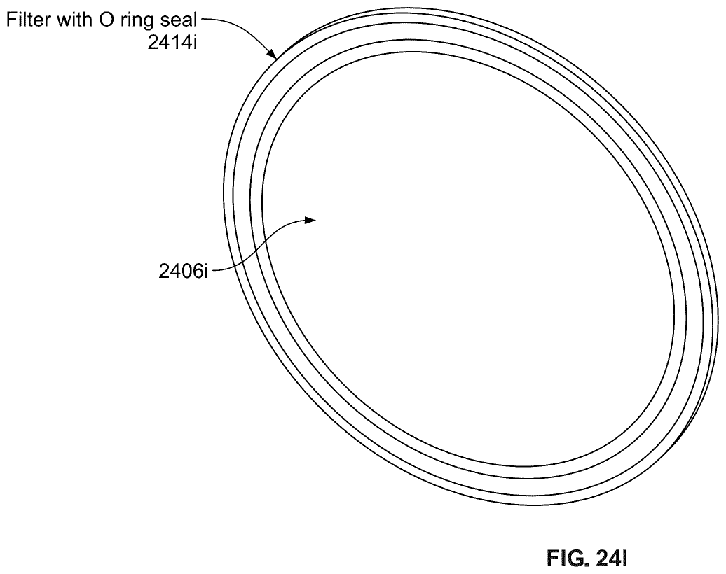

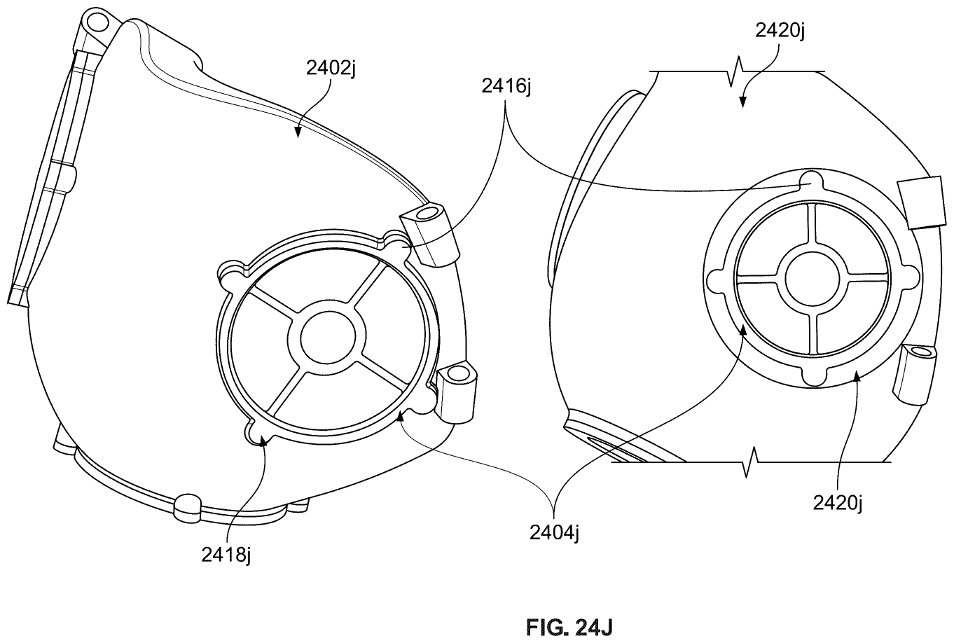

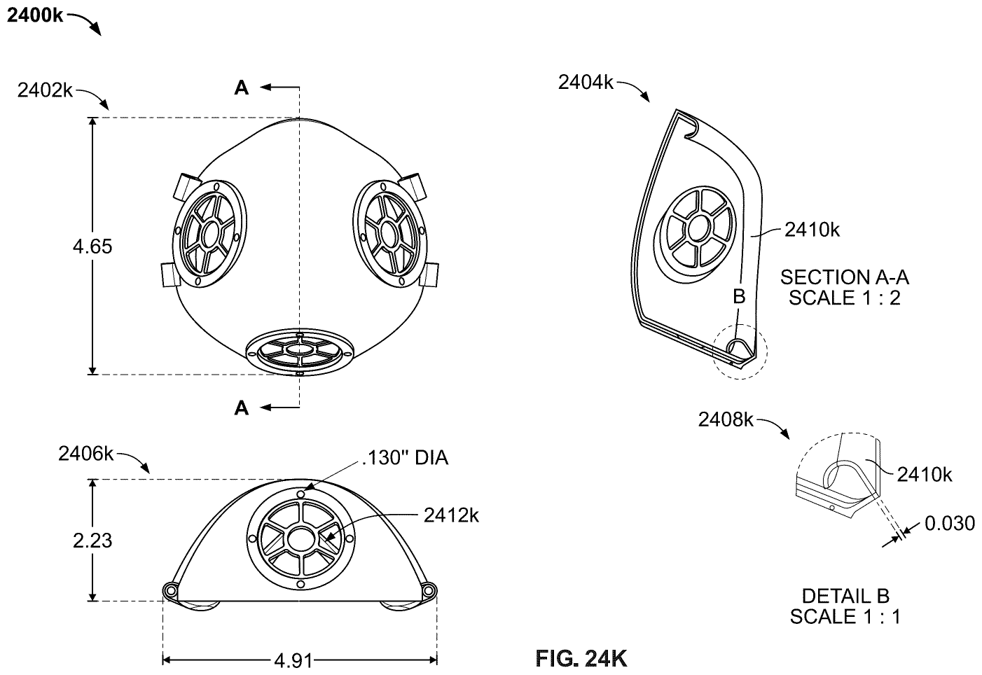

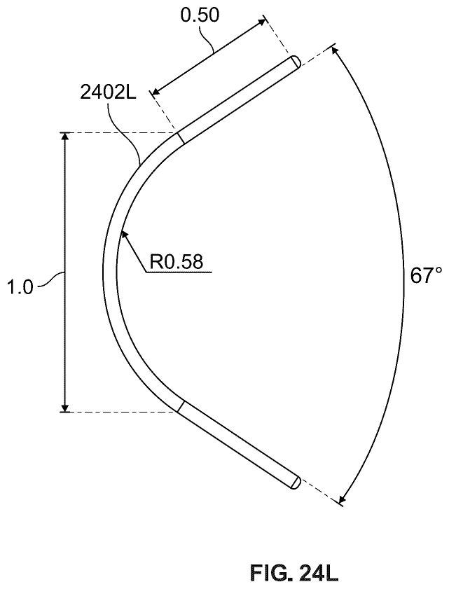

FIG. 24A illustrates a front top perspective view of a three-port mask 2400a in accordance with some embodiments of the present specification. FIG. 24B illustrates a front perspective view of the three-port mask 2400a in accordance with some embodiments of the present specification. M ask 2400a includes two ports 2402a and 2404a on the sides, with one port on either side, and a third port 2406a at the bottom side. In some embodiments, ports are circular in shape. In other embodiments, ports of different shapes are provided, and discussed subsequently. Each port is covered with a web, net or a mesh insert 2408a that supports a filter within the port. In some embodiments, each insert 2408a is made from polycarbonate material. Each port, also herein referred to as an opening or window, 2402a, 2404a, and 2406a, is covered with a filter (not shown). The filter port or filtration area diameter is 40 mm (and an area of approximately 5 square inches), and 45 mm (and area of approximately 6.90 square inches), in different embodiments. In some embodiments, the filter port or filtration area is in a range of 4.5 to 7 square inches. In embodiments, pleated filters are used wherein an area of the actual pleated filter is less than or greater than the area of the filter port or filtration area. In embodiments, the filter port or filtration area houses a filter with a surface area ranging from 50% of the filtration area to 5,000% of the filtration area. Further, in embodiments, mask 2400a includes multiple loops 2410a. Two loops 2410a are provided proximal to the outer edges of side ports 2402a and 2404a, on the surface of body of mask 2400a. Each loop is configured to accommodate an end of a strap that is used to wear the mask 2400a.

In embodiments, the insert is attached to a mask body using magnets. FIG. 24E illustrates a magnetic insert 2404e, which is circular in some embodiments. In alternate embodiments, the form of the magnetic insert 2404e is of any other shape. An internal circumferential surface of the insert 2404e may include multiple magnets that are equally spaced from adjacent magnets. The insert 2404e itself may be made from a polycarbonate material. FIG. 24F illustrates a side perspective view of a mask body 2402f with a magnetically attached insert 2404f, in accordance with some embodiments of the present specification. Magnets 2408f are attached to the internal circumferential surface of insert 2404f, and are attracted to the circular rim of each opening on external surface of mask body 2402f. The figure also shows a filter 2406f inserted within insert 2404f. In some embodiments, the filter 2406f is fitted with an O-ring seal on an internal side of insert 2404f. While the embodiments are described with reference to O-ring seals, the seal may be of any closed-ring shape, suitable for the shape of the opening. FIG. 24G illustrates a front view 2430g, a cross-sectional view 2432g, and a back view 2434g, of an exemplary insert 2404g with magnets 2408g. FIG. 24H illustrates a side elevated view 2408ha and a top view 2408hb of a magnet 2408f of FIG. 24F. In some embodiments, the magnet 2408f has a diameter of 1/8th of an inch and is made using Neodymium. FIG. 24I illustrates a filter 2406i with an O-ring seal 2414i. FIG. 24J illustrates another embodiment of an insert 2404j that includes at least four protrusions 2416j from four equally spaced edges of circumference of the circular insert 2404j. Each protrusion 2416j may include a magnet that conforms to a surface outside the port of a mask body 2402j. In some embodiments, each protrusion 2416j is met by an equivalent or similarly sized and shaped elevated protrusion 2418j on a perimeter of the opening on the body of mask. In some other embodiments, an elevated boundary 2420j encircles the opening, and the protrusions 2416j are placed anywhere on the boundary 2420j on the body of the mask.

FIG. 24K illustrates additional views of the mask 2400k of FIGS. 24I and 24J, along with exemplary dimensions of some of the components. A front view 2402k of mask 2400k illustrates a length at the central longitudinal axis, extending from a top to a bottom of the mask 2400k, which is approximately 4.65 inches. A section view 2404k along the same axis illustrates a tube-like structure 2410k on an inner perimeter of the mask 2400k. In some embodiments, a wire is inserted inside the structure 2410k to support the shape of mask 2400k. A bottom view 2406k illustrates a third opening 2412k on a bottom surface of mask 2400k. A depth of the mask extending from one end to another along a central axis on the bottom surface of mask 2400k, is approximately 2.23 inches, which covers the area under the chin of a face of the user wearing the mask 2400k. An enlarged view 2408k of a section of the structure 2410k illustrates a thickness of the cover on structure 2408k to be approximately 0.03 inches.

FIG. 24L illustrates an exemplary wire form 20421 of a face mask of FIG. 24K, in accordance with some embodiments of the present specification.

FIG. 24C illustrates a top view of a mask 2400c in accordance with some embodiments of the present specification. A seal 2412c is attached to the inner edge of mask 2400c. FIG. 24D illustrates a seal 2412d without the mask. Seal 2412c/2412d is provided to encompass the complete rim formed by an inner edge of body of mask 2400c. The seal 2412c/2412d contacts the surface of a wearer's face who wears the mask 2400c. In embodiments, seal 2412c/2412d is approximately 5 to 25 mm wide extending perpendicular to the mask's edge, towards the side that will contact wearer's face. In some embodiments, the body of the mask and the seal are composed of the same material. In other embodiments, the body of the mask and the seal are composed of different materials. In embodiments, the seal 2412c/2412d is made from a relatively soft material compared to body of mask 2400c. In various embodiments, the seal material comprises silicone, rubber, non-latex rubber, or a thermoplastic elastomer. In various embodiments, the seal material has a durometer rating that is less than a durometer rating of the body material. In embodiments, the durometer rating of the seal material is in a range of 30D to 80D.

M asks of FIG. 21A to 24F, and other embodiments illustrated in the present specification, are configured in different sizes. In some embodiments, the masks of the present specification are configured in a small size for small-sized faces, a medium size for medium-sized heads, and a large size for large-sized heads. In embodiments, the sizes of the masks may vary from extra small to small to medium to large, wherein each size is associated with a height and/or a width. In embodiments, the height is measured from the bottommost portion of the mask to the topmost portion of the mask, along a straight vertical distance. In embodiments, the width is measured at the maximum width of the mask, along a straight horizontal distance. In embodiments, the width of the mask ranges from 55 mm to 115 mm. In embodiments the height of the mask ranges from 55 mm to 115 mm. In embodiments, the mask has a width of 55 mm and a height of 55 mm. In embodiments, the mask has a width of 65 mm and a height of 65 mm. In embodiments, the mask has a width of 85 mm and a height of 85 mm. In embodiments, the mask has a width of 115 mm and a height of 115 mm. Small-sized masks may be appropriate for children of different ages, which larger sizes may be suitable for adults, or teens with faces of different sizes.

FIG. 25A illustrates different face sizes 2502 and a table 2504 showing common face widths in millimeter (mm) that correspond to common face lengths (in mm). In embodiments of the present specification, mask designs (bladder soft seal/malleable perimeter wire/flexible mask body/strap attachment design) allows for a mask fit to NIOSH ASTM standard F3407 in face lengths 98.5 mm-138.5 mm and face width of 120.5 mm-158.5 mm in more than 50% of users without needing sizing using a formal fit test.

FIG. 25B illustrates an anatomy of a human face, and specifically an anterior view of the superficial facial muscles of a human face. The various embodiments of the mask in accordance with the present specification are configured with a transparent surface configured to flexibly seal against a skin surface of the user. In embodiments, flexibly seal refers to a perimeter of the transparent surface positioned on the skin such that no airflow is permitted to pass across the sealed perimeter. The skin surface is positioned over at least a portion of the user's zygomaticus major muscle 2502b, at least a portion of the user's zygomaticus minor muscle 2504b, all of the user's orbicularis oris muscle 2506b, at least a portion of the user's risorius muscle 2508b, all of the user's depressor muscle 2510b, and all of the user's mentalis muscle 2512b. A non-filtration surface of the transparent surface of the mask is also configured to be positioned at least a distance of +/−10 mm from the junction of the bony and cartilaginous parts of the nose. A bottom of the mask is configured to be positioned under the chin (between the tip of the chin to the junction of bottom of the face and the neck). The non-filtration surface is also non-porous so that the surface does not permit any air flow to pass through the surface.

### Filter

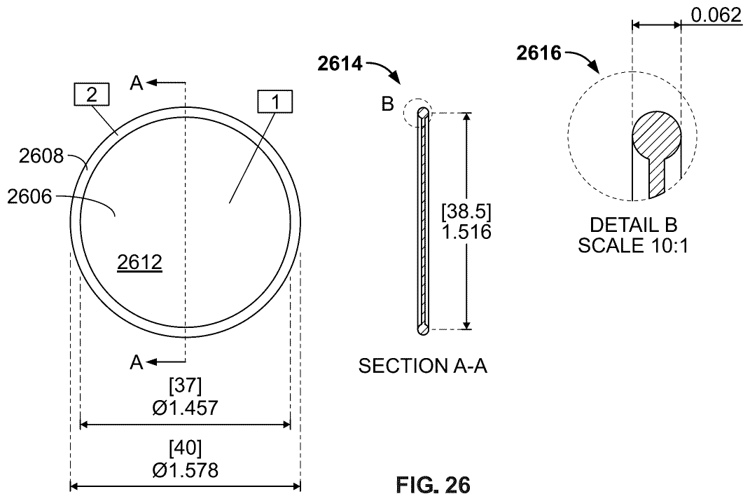

FIG. 26 illustrates an exemplary front view 2612, a cross sectional view 2614, a detailed view of an edge of cross-sectional view 2616, of a filter 2606, in accordance with some embodiments of the present specification. In some embodiments, the filter 2606 is made from fabric suitable for an N95 filter. An edge 2608 of filter 2606 is made from silicone, and has a width of approximately 0.062 inches. The edge 2608 is configured as a closed-ring to fit the filter 2606 within an opening/port/window of a mask body. An inner diameter of the filter 2606 is approximately 1.457 inches, and an outer diameter, including the edge 2608, is approximately 1.578 inches.

In embodiments of the present specification, the filters are configured to be of medical-grade efficacy. A material used in the filter is compatible with three or four ply construction of N95 or PM 2.5 masks. In embodiments, a total surface area of a filter is >5 inch². In embodiments, handling and sealing of each filter is enhanced by an O-ring which enables a firm seal. While the specification is described with reference to an O-ring, the shape of the seal could be any closed-ring shape that is suitable to cover the opening meant for the filter. O-ring made from biocompatible soft silicone is preferably used. The filter is configured to be breathable and meets NIOSH inhale/exhale specifications, such that a pressure difference achieved is <25 Pa. In embodiments, the filter has an anti-microbial coating, such as Ag/ZnO, or other coating. In some embodiments, the filter material has desiccating properties.

Following table summarizes test results for filter material:

| | | | | | | |
|---|---|---|---|---|---|---|
| | | | Standard | | | Test Method/ |
| Test Items | Description | Unit | Requirement | Results | Conclusions | Remarks |

1 White Non-woven Fabric 17.5 × 200 cm

| | | | | | | |
|---|---|---|---|---|---|---|
| Pressure Difference | / | Pa | ≤49 | 22 | Pass | YY 0469-2011 |
| (BFE) | / | % | ≥95 | 1: >99.99 | Pass | YY 0469-2011 |
| Bacterial Filtration | | | | 2: >99.99 | | |
| Efficiency | | | | 3: >99.99 | | |
| (BFE) | | | | | | |
| (PFE) | / | % | ≥30 | 1: 99.95 | Pass | YY 0469-2011 |
| Particle Filtration | | | | 2: 99.97 | | |
| Efficiency | | | | 3: 99.96 | | |
| (BFE) | | | | | | |

In some embodiments, the filter material achieves a filtration efficiency ≥99% against a sodium chloride (NaCl) aerosol challenge with a count median diameter of 75±20 nm and a geometric standard deviation of 1.86 at a flowrate of 85±4 Lpm at a face velocity of 10 cm/sec. The filter material also achieves inhalation airflow resistance of ≤15 mm H2O at a flowrate of 85 ±4 L pm at a face velocity of 10 cm/sec. In some embodiments, a face mask composed of filtration surfaces (<75% of mask surface area) and non-filtration surface (>25% of mask surface area), wherein the filtration surface has a filtration efficiency ≥95% against a sodium chloride (NaCl) aerosol challenge with a count median diameter of 75±20 nm and a geometric standard deviation of 1.86 and an inhalation airflow resistance of ≤15 mm H₂O at a flowrate of 85±4 LPM and at a face velocity of 10 cm/sec, the mask results in an average reduction of inhaled bioaerosol of <10 microns by >33%.

In embodiments of the masks formed by creating air shields, the wearable face structure is configured to weigh <250 gms and is composed of air delivery ports covering ≤95% of the surface area wherein the face structure has a clean air delivery rate of ≥30 LPM and a noise level of ≤65 dB and results in an average reduction of inhaled bioaerosol of ≤10 microns by ≥33%. In some embodiments, a wearable face structure does not form or require an airtight seal around a patient's nose or mouth like a mask.

### Bladder/Seal

In embodiments, a bladder-like facial seal is configured to achieve same or higher decrease in aerosol inhalation when compared to an N95 mask. The seal is configured from a material that is same as the mask body to enable a single mold for manufacturing. The seal is made from a biocompatible material such as food or FDA grade silicone. In embodiments, a seal is easily formed when wearing mask with strap and is maintained with facial movements. A wearer of the mask with the seal of present specification stays comfortable for at least twice the hours longer than a user wearing an N95 mask. In embodiments, the mask has a flexible perimeter seal that fits >50% of face structures across two different ISO digital head forms created using NIOSH Anthropomorphic Data. The flexibility of the seal ensures that a perimeter of the transparent surface is positioned on the skin such that no airflow is permitted to pass across the sealed perimeter.

### Strap

In embodiments, a commercial off-the-shelf strap is used, which is made from a stretchable material. The strap configuration allows the mask to hang around the neck when not in use. The embodiments of present specification allow multiple strap configurations to be used with the various mask embodiments. The various configurations include two strap configuration, bungee configuration, unibody configuration, ear loop configuration, among other configurations. Head and ear straps, or a combination of both, is designed for >1,000 on/off uses. The embodiments offer ease of attachment/detachment of the strap to/from the mask (if needed). Embodiments of the strap meet ASTM F3407 specifications, which are currently met by N95 mask only. The strap configurations of the present specification are compatible with various hair styles and type (long, short, smooth, coarse, wavy, straight).

### A Esthetic Considerations and Packaging

In embodiments, filter assembly of the present specification is flush with the mask body so that there are no protrusions on the mask surface and a cover can be placed inside the mask. The masks of present specification have a smooth contour and is more aesthetic than a “gas mask” or a “jock strap”.

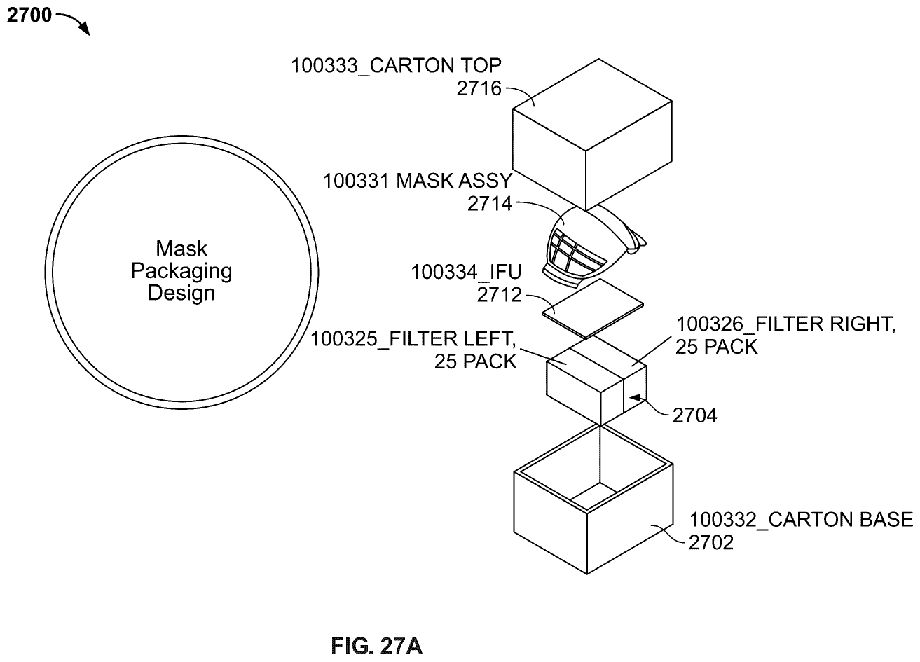

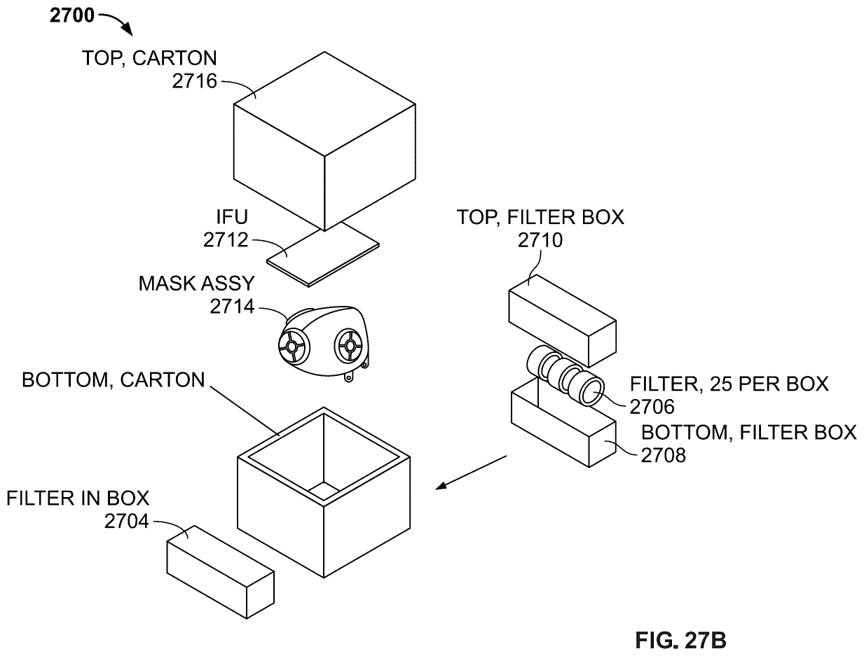

FIG. 27A illustrates a break-out view of a mask packaging design, in accordance with some embodiments of the present specification. FIG. 27B illustrates another view of the break-out of components within the mask packaging design of FIG. 27A, additionally showing the components of one filter box and its contents. Referring simultaneously to FIGS. 27A and 27B, the packaging 2700 includes a carton base 2702 containing at least two symmetrical boxes 2704 to store filters. In some embodiments, each box is provided with 25 filters 2706. The boxes 2704 comprise a bottom filter box 2708 that holds the filter 2706, and a top filter box 2710 that covers the bottom box 2708. Above the boxes 2704, an IFU 2712 is placed in a flat position. Above IFU 2712, a mask assembly 2714 is kept, which includes the mask body with wires and inserts among other components of the mask. In some cases, the IFU 2712 is placed above the mask assembly 2714. Finally, a top carton 2716 is placed to cover the carton base 2702 and its contents. In embodiments, the masks are packaged aesthetically and in a format that is robust for storage and protection during transportation. The packaging includes six-month supply of filters with each mask. In some embodiments, each filter box includes about 25 filters. The packaging is modern and minimalistic. In embodiments, the masks of the present specification can be used for six or more months. The masks are washable with soap/water and can be reused.

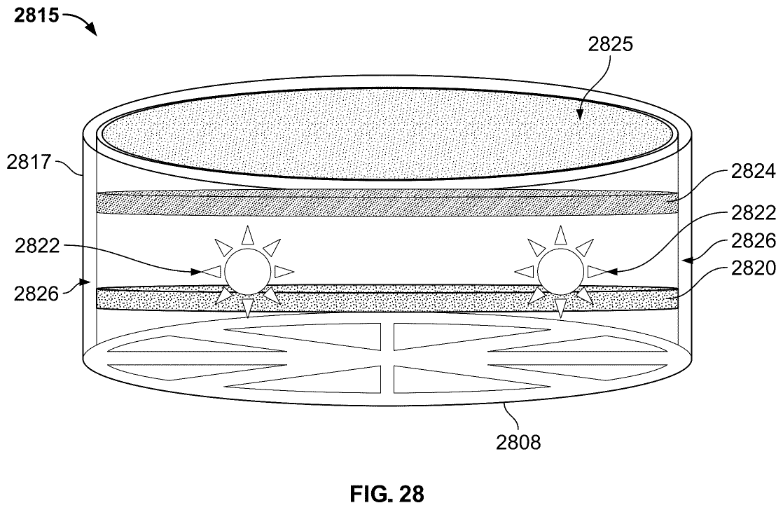

FIG. 28 illustrates an exploded view of a filter with layers. As shown in FIG. 28, in some embodiments, the air intake blower or pump 2808 is in fluidic communication with a proximate attachment 2815 that includes an ultra-violet (UV)/Photocatalytic Oxidation (PCO) filter. Filters are placeholders for filtration that can combine mechanical filtration, ventilation, and destructive filtration. In some embodiments, the air passing through the filter is exposed to UV or an ionizer. In embodiments, filter attachments 2815 fit into the filter area of a mask.

As shown, the attachment 2815 has a generally cylindrical housing 2817 that incorporates a first TiO₂ filter 2820, one or more UV lights sources 2822 such as, for example, LEDs generating light in the wavelength range of 210 nm to 495 nm, a second charcoal filter 2824 followed by a third particulate filter 2825 that meets at least the U.S. National Institute for Occupational Safety and Health (NIOSH) N90 classification of air filtration. In some embodiments, the internal surfaces of the housing 2817 has UV reflective coating 2826 having reflectivity in the range of 25% to 99%.

### PATIENT/PROCEDURAL MASK

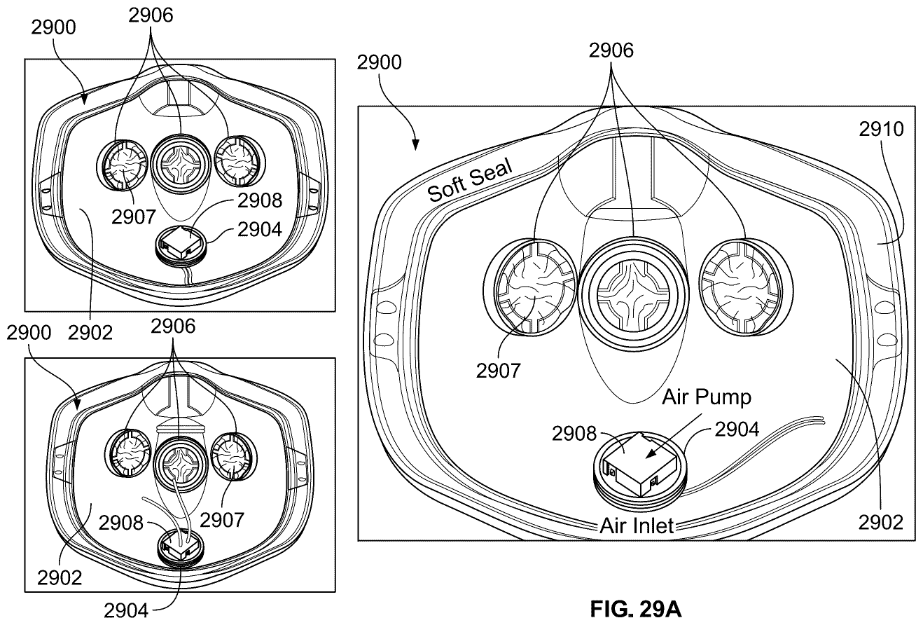

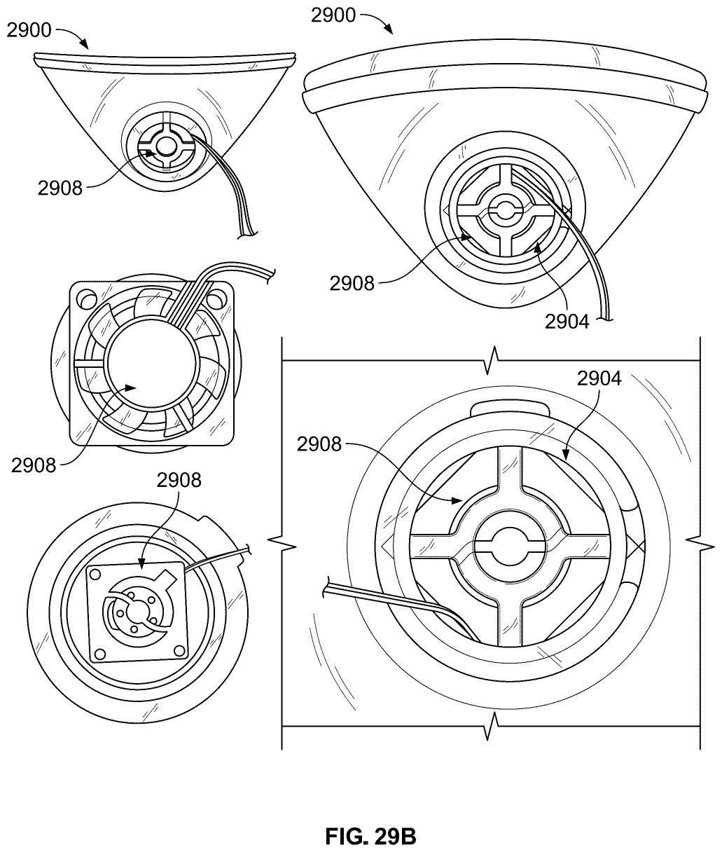

FIG. 29A shows a first view 2901, a second view 2903, and a third view 2905 of a positive air pressure (PAP) mask 2900, in accordance with some embodiments of the present specification. As shown, the mask 2900 includes mask body 2902, at least one air inlet 2904, and a plurality of air outlets 2906. In some embodiments, the at least one air inlet 2904 and each of the plurality of air outlets may include a filter 2907 having a specification of PM 2.5 (or filters 95% of particulate matter above 0.3 μ). In some embodiments, the mask 2900 includes three air outlets 2906. In some embodiments, the at least one air inlet 2904 and each of the plurality of air outlets 2906 have at least one dimension (such as, for example, a diameter where the inlet and outlets are substantially circular) greater than 25 mm.

In embodiments, the at least one air inlet 2904 incorporates an air intake blower or pump 2908 that modulates the flow and pressure of air entering the mask from the at least one air inlet 2904 and exiting through the plurality of air outlets 2906. In embodiments, the air intake blower or pump 2908 enables modulation of the flow of the air from the at least one air inlet 2904 to maintain a therapeutic positive pressure (within the mask) that is transmitted to a patient's airway to maintain a patent airway. In some embodiments, the air intake blower or pump 2908 is in data communication with a processor that executes a plurality of instructions or programmatic code (an event detection software) to increase the pressure gradually in response to air flow changes until adequate patency is detected. In some embodiments, the air intake blower or pump 2908 enables generation of a lowest pressure, within the mask, that is necessary to maintain upper airway patency. In embodiments, the air intake blower or pump 2908 changes the flow of air to change a pressure in the mask during inspiration to assist with an inspiratory function of the patient. In embodiments, the air intake blower or pump 2908 changes the flow of air to change a pressure in the mask during expiration to assist with an expiratory function of the patient.

In embodiments, the plurality of air outlets 2906 resist the outflow of air and the resistance of the outlets determines the positive pressure within the mask 2900. In some embodiments, a resistance of the outflow of air through each of the plurality of air outlets 2906 can be modulated to change the pressure in the mask. In some embodiments, the resistance of the outflow of air through each of the plurality of air outlets 2906 is controlled by changing the resistive filter 2907. In some embodiments, the resistance of the outflow of air through each of the plurality of air outlets 2906 is controlled by changing a cross-sectional dimension, such as a diameter, of each of the plurality of air outlets 2906.

When worn by a patient, the rim 2910 of the mask 2900 forms a soft seal with the face of the patient. In some embodiments, the seal is formed to withstand, within the mask 2900, a pressure ranging from 1 cm of H₂O to 50 cm of H₂O. In some embodiments, the seal withstands a pressure ranging from 2-25 cm of H₂O. In some embodiments, the seal withstands a minimum pressure of 5 cm H₂O and a maximum pressure of 20 cm H₂O.

FIG. 29B shows various views of the air intake blower or pump 2908 positioned in the at least one air inlet 2904 of the mask 2900. Additional embodiments of air purifier/blower/pump configurations have also been described above in the context of FIGS. 24 to 26, which should also be considered here.

Referring back to FIGS. 29A, 29B, in some embodiments, at least 50% or more of the inhaled air enters through the at least one inlet 2904. In some embodiments, an optional fan or blower is attached to one or more of the plurality of air outlets 2906 to vent air from the mask. In some embodiments, at least 50% or more of the air exits through the plurality of air outlets 2906.

In some embodiments, one or more sensors are positioned within the mask 2900 to measure one or more respiratory parameters and to control one or more functions of the air intake blower or pump 2908. In some embodiments, the mask 2900 includes a plurality of sensors configured to monitor the patient's health or deliver therapy to the patient. In various embodiments, the plurality of sensors include those related to determine EKG, EEG, oxygen saturation, eTCO2, pressure, detect sound (microphone), and detect traces of alcohol.

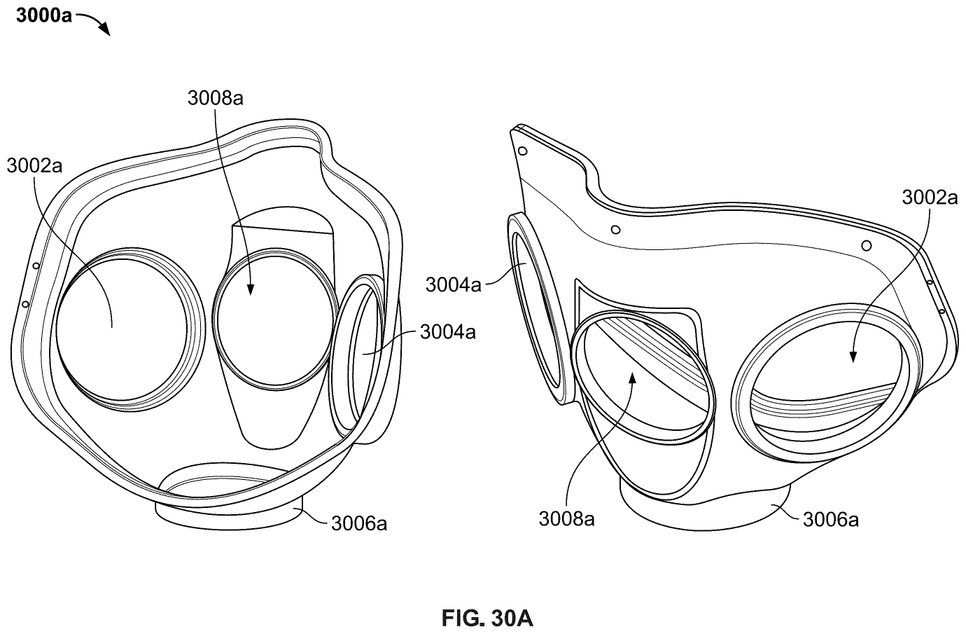

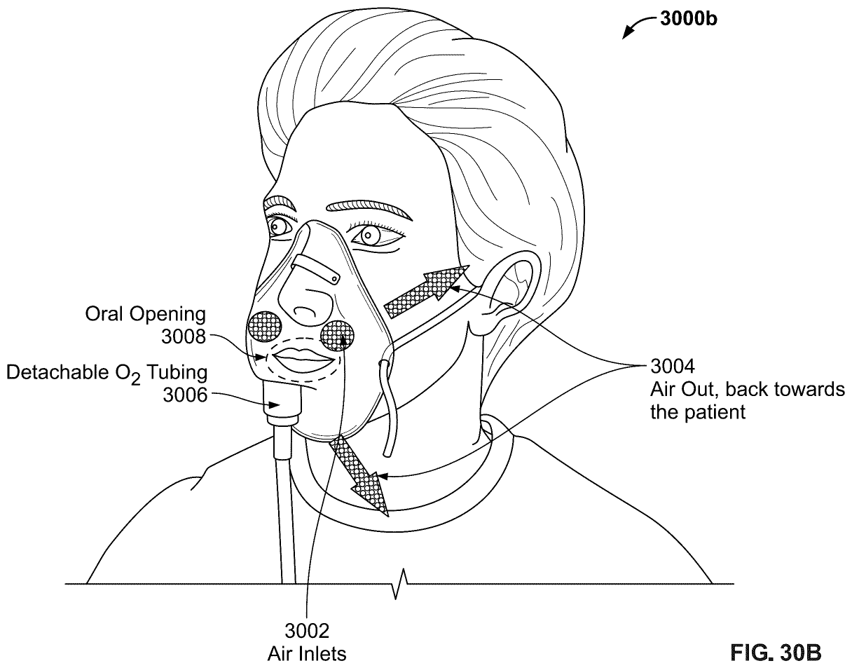

FIG. 30A is an illustration showing different views of a three-port mask 3000a in accordance with some embodiments of the present specification. The illustration shows two openings 3002a and 3004a on the surfaces of the left side and the right side of the mask 3000a; and a third opening 3006a on a bottom side surface. In embodiments, the bottom opening 3006a is optionally used for attaching tubing, such as to supply oxygen. The openings 3002a and 3004a on the sides are optionally for placing filters and provide air inlets. A front oral opening 3008a is also provided for accommodating oral instruments. FIG. 30B is an illustration of a user/patient wearing three-port mask 3000b in accordance with some embodiments of the present specification, to prevent airborne pathogen droplet spread. The various embodiments of masks, described in accordance with the present specification are beneficial for essential services workers, and in particular, healthcare workers. In embodiments, mask 3000 includes air inlets 3002, air outlets (towards the back of the user) 3004, a detachable tubing (for oxygen) 3006, and an oral opening 3008. In some embodiments, mask 3000 is worn by a patient during a surgical procedure, such as an endoscopy.

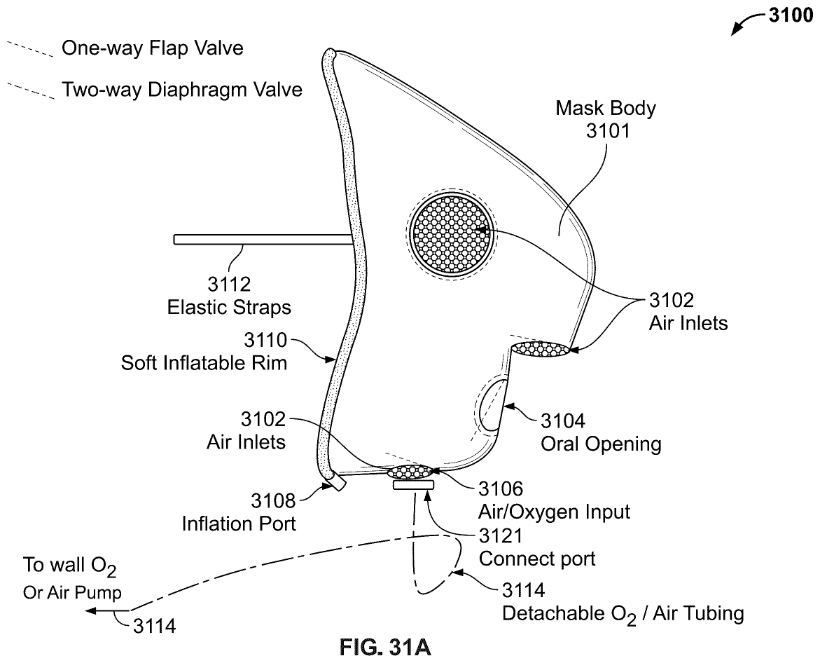

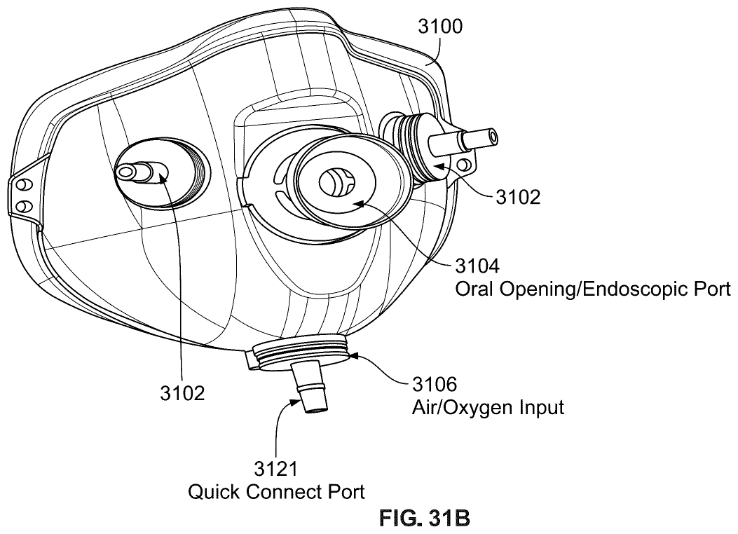

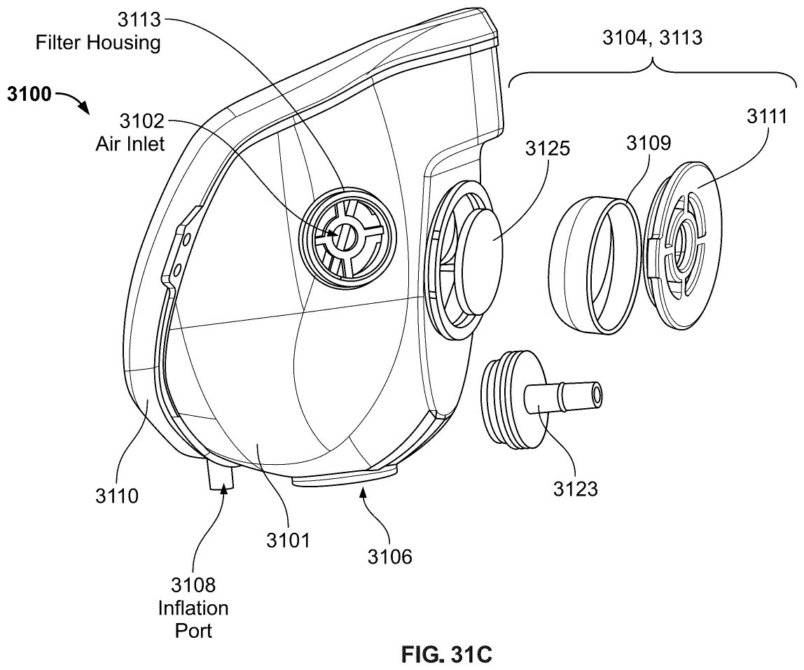

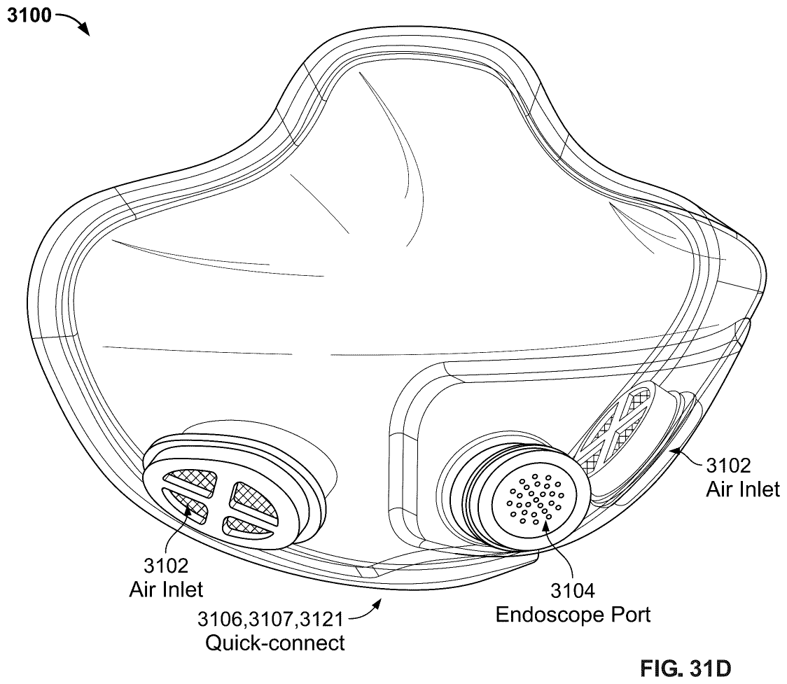

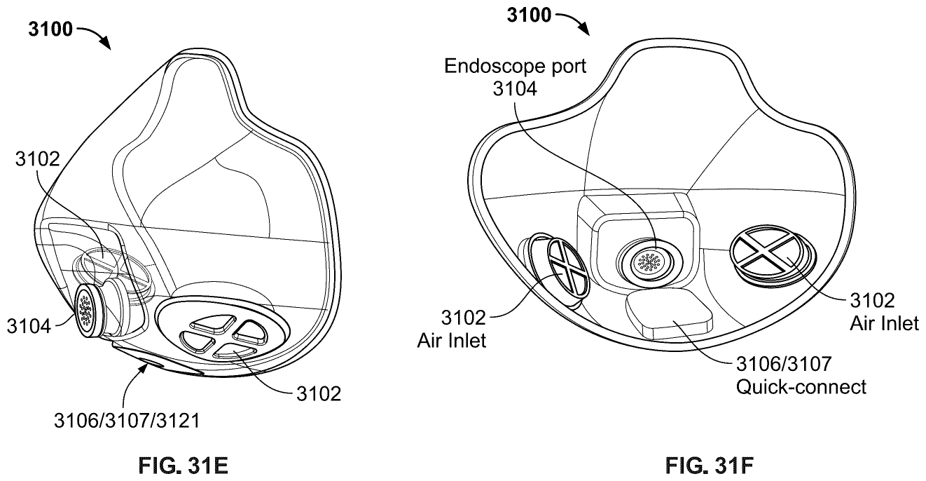

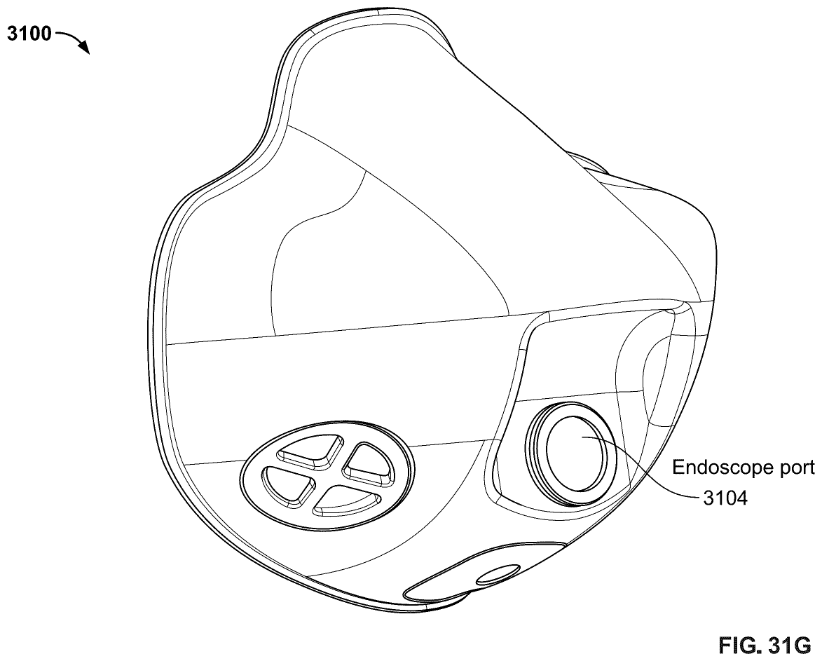

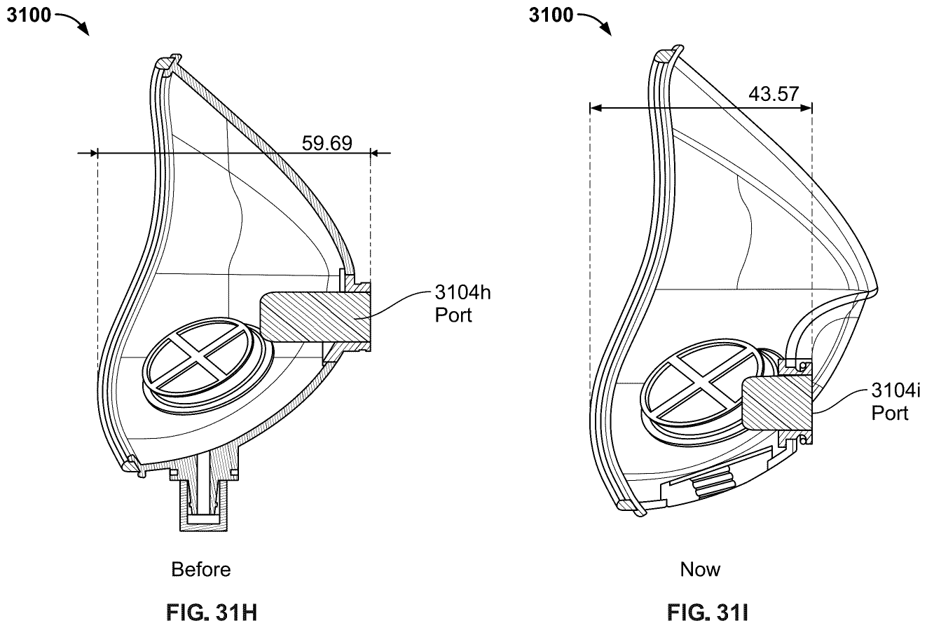

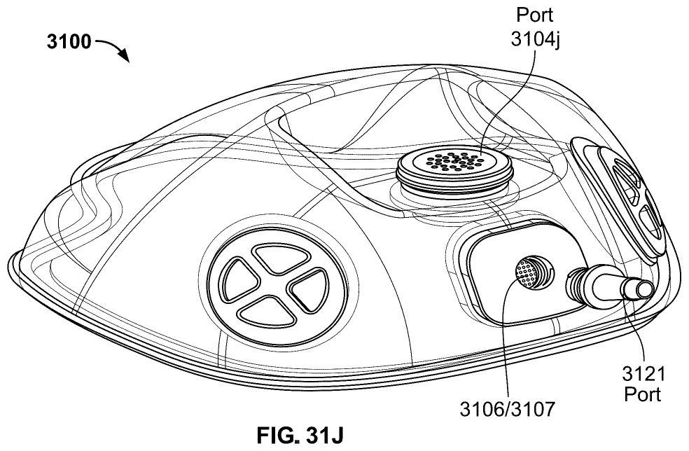

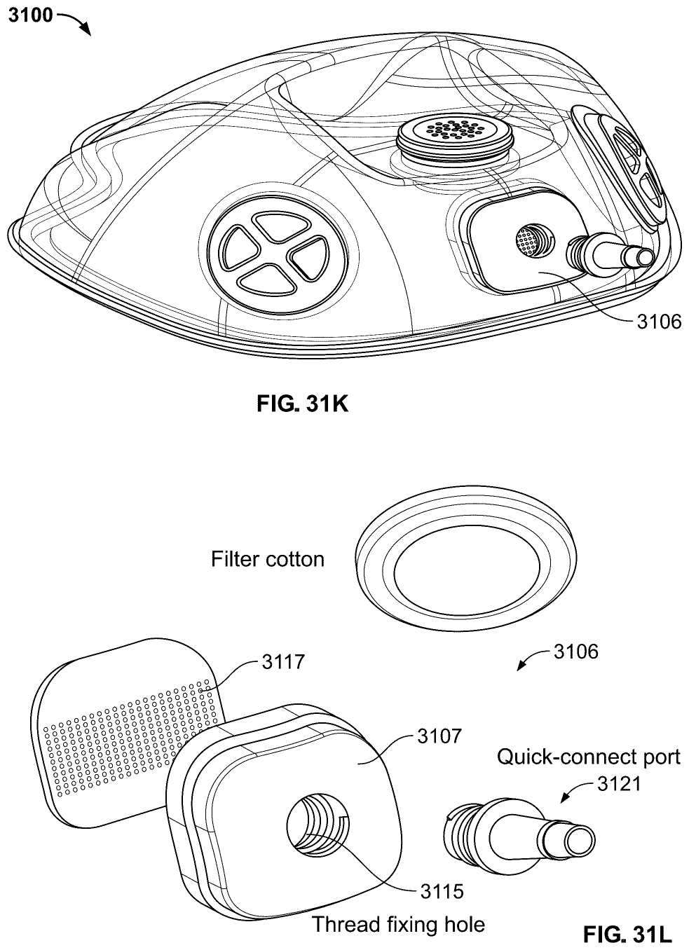

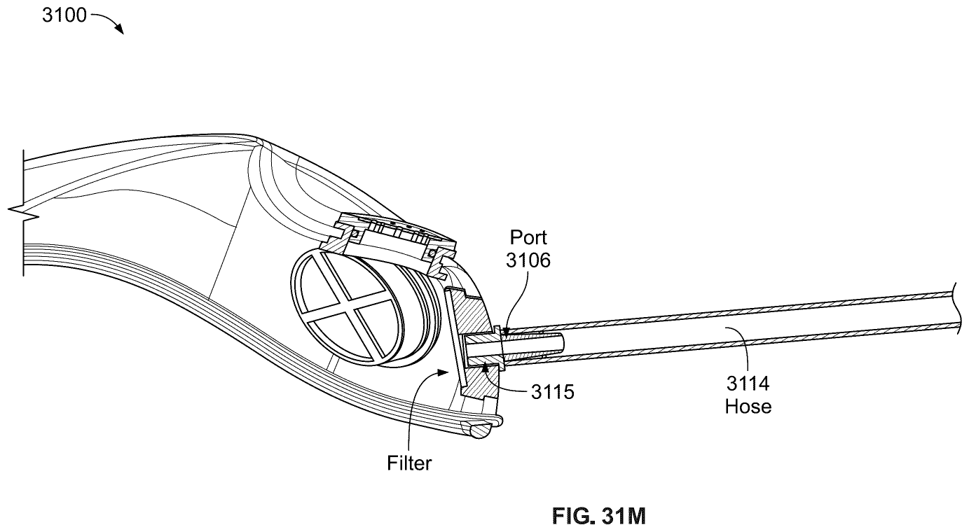

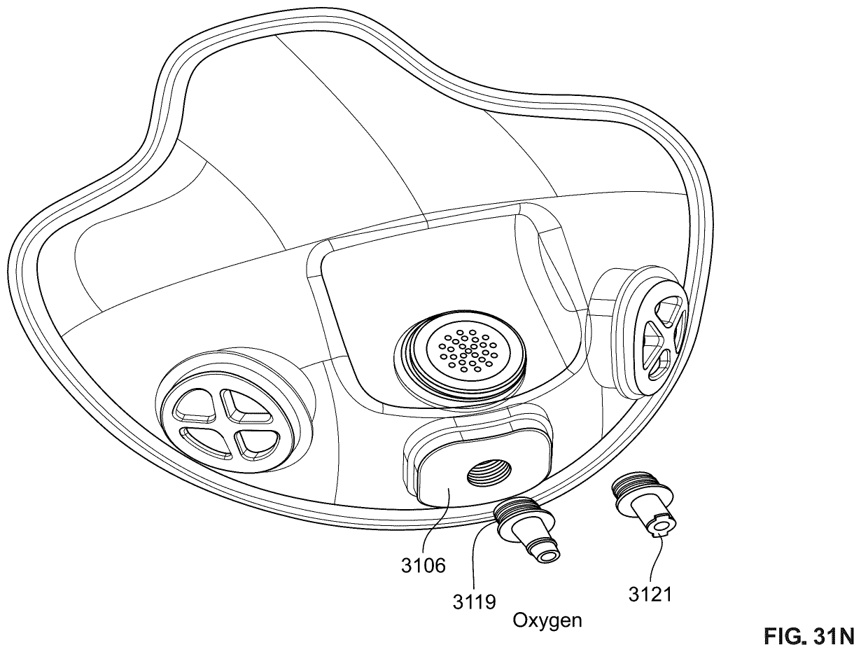

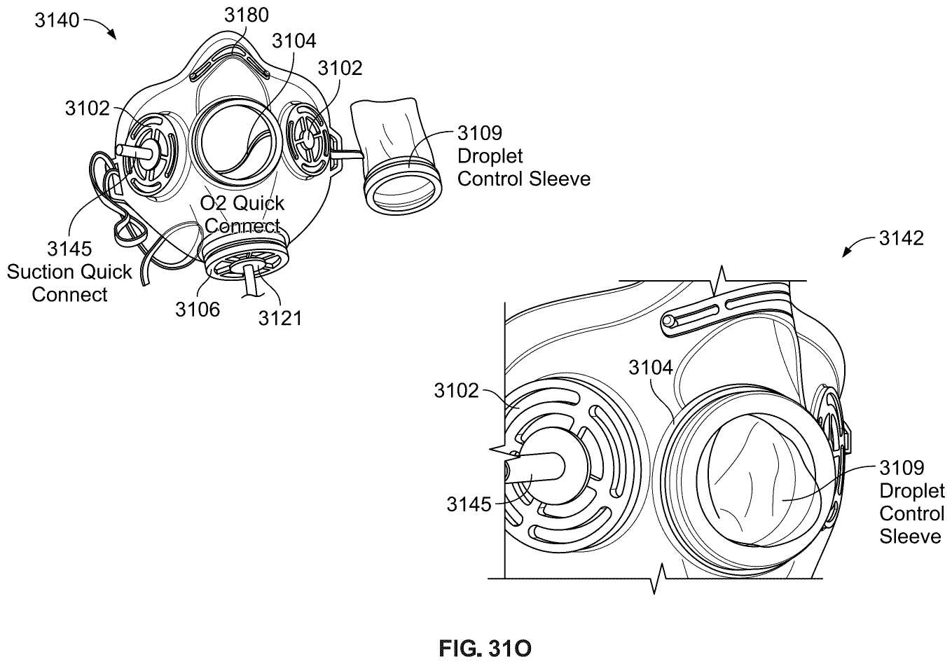

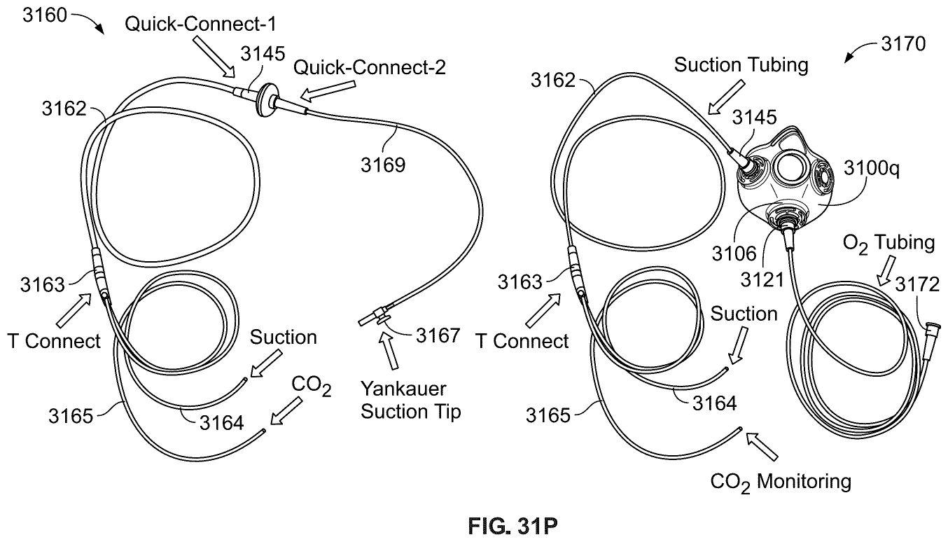

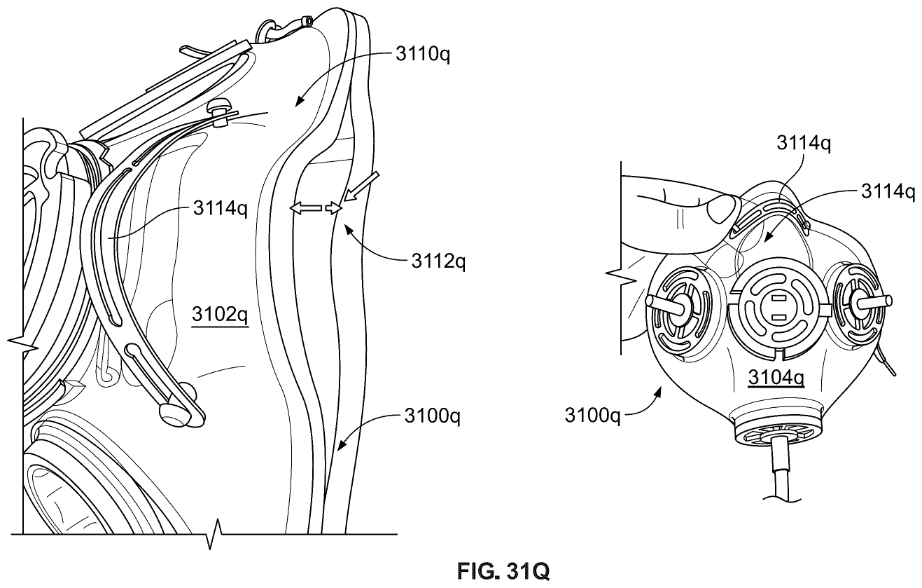

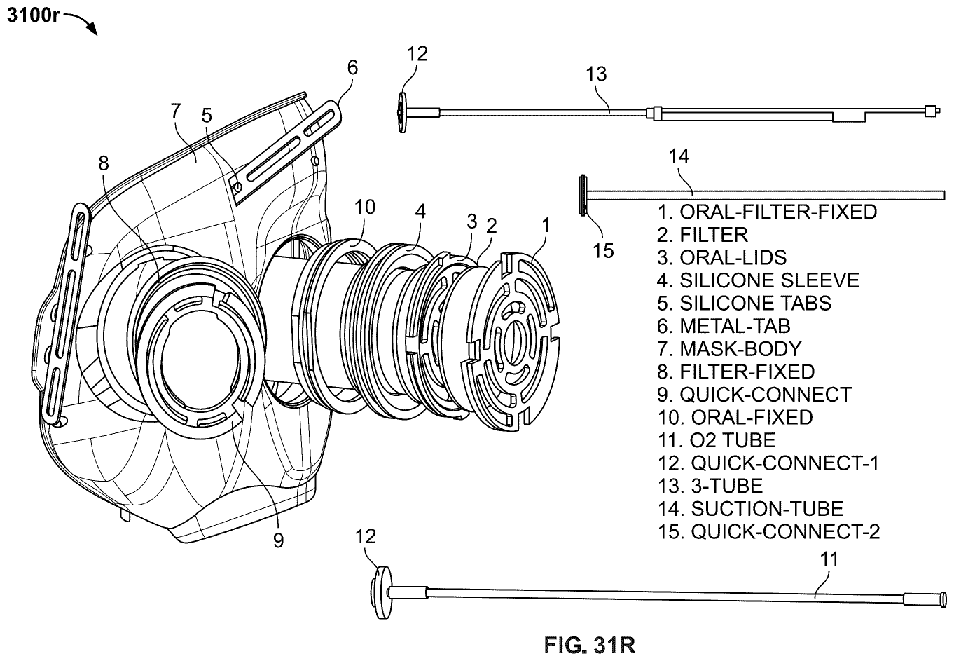

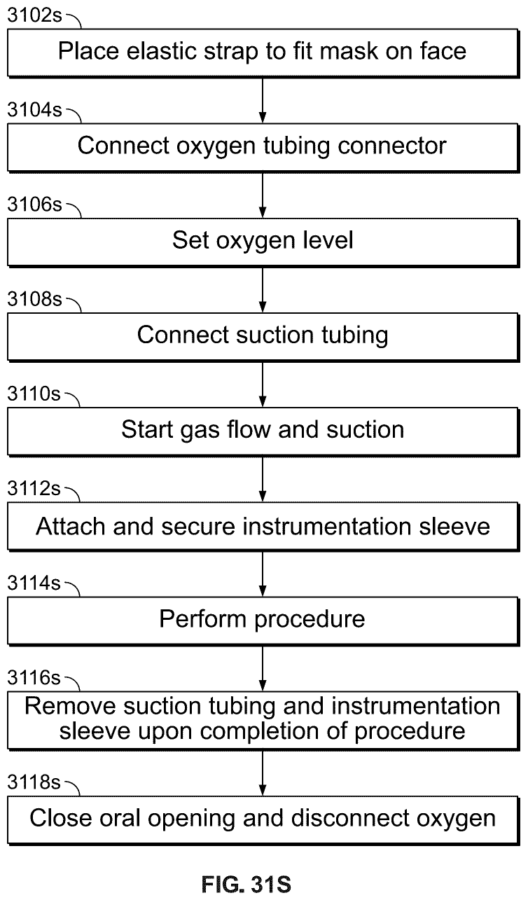

FIG. 31A is a side elevation view of the mask shown in FIG. 30B. As shown in FIG. 31A, mask 3100 includes air inlets 3102 that comprise a valve that allows for air entry into the mask during breathing but restricts droplet flow out of the mask during breathing, coughing, or sneezing. The air inlets 3102 include replaceable filters that can be incorporated therein using a filter housing and associated components, as further described below. In addition, mask 3100 includes an oral opening/valve 3104 which is comprised of a two-way diaphragm valve that allows for endoscopic insertion and a one-way flap valve to prevent any aerosolized oropharyngeal content from escaping the mask during coughing or sneezing. It should be noted herein that the valve configurations are not limiting and that other valve configurations may be employed to achieve the same function. Mask 3100 also includes an air/oxygen input 3106 that further includes an inlet valve and a filter housing (shown in FIG. 31C) and associated filter components and is optionally equipped with a tubing connector/quick connect port 3121 located below the oral opening 3104 such that it does not interfere with instrument/endoscopic insertion. Thus, the patient can be disconnected from wall oxygen while keeping the mask on for ambulation. Tubing 3114 may be connected to an air pump or oxygen, such as wall oxygen or an oxygen tank. An inflation port 3108 is provided and integrated with a self-adjusting, soft inflatable rim or bladder 3110, which can be inflated with air or water for a soft seal around the mask through the inflation port 3108. In embodiments, soft silicone rubber rung structures 3110 may be used to create the soft seal. In embodiments, mask 3100 includes elastic straps 3112 for fastening the mask to a patient.

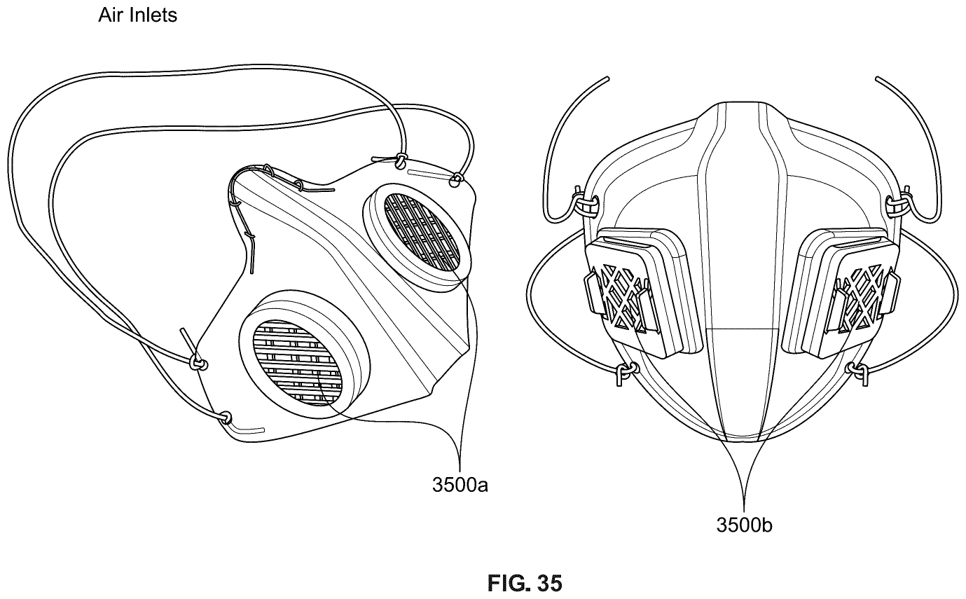

In embodiments, the soft inflatable rim 3110 may be pre-inflated with fluid. In a fluid-filled bladder embodiment, fluid may occupy 5% to 95% of the bladder volume, at a pressure at or below atmospheric pressure. Preferably, pressure is applied equally to all areas where the self-fitting bladder rim or soft inflatable rim contacts a person's face. In embodiments, the soft inflatable rim 3110 may be inflated with air via a pump mechanism to inflate the rim of the mask for a custom, comfortable yet tight fit. In embodiments, the snug fit ensures N-95 standard fit capabilities. In embodiments, the pump is a bladder type pump (such as a Reebok Pump). In embodiments, the pump may sit at or under chin level for pumping air into the rim via the inflation port 3108. In embodiments, the soft inflatable or self-fitting rim has an initial first shape and conforms to a second shape after worn by a person to self-adapt to a contour of a person's face, creating an airtight seal, thus not allowing air to enter into the inside of the mask from around the rim of the mask. In embodiments, a formal “fit test” is not required and the user does not need to adjust the shape of the mask during wear. FIG. 35A, described in detail below, includes steps for a method for sizing a mask without the need for a fit test. In embodiments, force is applied on an area greater than the mask body thickness, thus distributing the force over a large area, distributing pressure on the person's face and reducing pressure points and associated discomfort. In embodiments, the mask body 3101 and soft inflatable rim or bladder 3110 are composed of different materials. In embodiments, the stiffness of the bladder material is less than at least one component of the mask body 3101. In embodiments, the soft inflatable rim or bladder 3110 material has a durometer rating less than a durometer rating of the mask body 3101.

FIG. 31B is a three-dimensional pictorial representation of the mask shown in FIG. 31A. As shown in FIG. 31B, mask 3100 includes air inlets 3102, oral opening or endoscopic port 3104, and an air/oxygen input 3106 that includes a filter housing (shown in FIG. 31C) and a tubing connector/quick connect port 3121. In various embodiments, all inputs/openings/ports (3102, 3104, 3106) are designed to receive at least one air filter. In some embodiment, suction can be applied to any of the ports to remove droplets created during various respiratory activities via suction. In another embodiment, any of the ports can be used to create access for instrumentation for naso-oropharyngeal procedures.

FIG. 31C is an exploded, perspective view of the mask shown in FIG. 31A. As shown in FIG. 31C, mask 3100 includes mask body 3101, one or more air inlet(s) 3102, which includes filters housed in filter housing 3113 as described above; inflation port 3108; soft inflatable rim 3110, which in an embodiment is a self-fitting rim; and oral opening or endoscopic port 3104 also comprising a filter housing 3113 and associated filters and ports. In an embodiment, the oral opening or endoscopic port optionally includes a droplet control sleeve 3109 with an opening ranging from 2 mm and expanding up to 36 mm to fit snugly around the shaft of an endoscope which expands with the passage of the endoscope. In embodiments, the droplet control sleeve is a replaceable, removable sleeve that may be connected to the filter housing 3113 to provide access to the nasal or oral passages via instrumentation. The sleeve is used to reduce the spread of droplets while allowing for the passage of air and instrumentation, such as but not limited to, an endoscope. In embodiments, the droplet control sleeve may be fabricated using filter material (such as N-95 or N-99) allowing for air to pass through the sleeve body without allowing droplets to pass. In various embodiments, the one or more ports can be configured to receive the droplet control sleeve to receive various instruments for medical/surgical procedures.

In embodiments, filter housing 3113 that is used with the oral opening 3104 includes a filter cap 3111 (such as, but not limited to a flap valve) that is used to cover the opening so that there are no aerosol leaks when the endoscope is removed or not present and also serves to secure the filter to the filter housing 3113. A filter connector or connector port 3123 is also provided and connects to the replaceable/removable filter 3125, housing 3113, or to the filter cap 3111. In embodiments, quick connect port 3123 connects to the rim of filter cap 3111 without interfering with the function of the filter. In embodiments, the connector port 3123 may be used to attach tubing for filtered air/oxygen administration, suction (such as suctioning droplets during a naso-oropharyngeal procedure, for example), among other functions or to attach additional filter components to the filter to augment the function of the filter. It should be noted that the filter housing 3113 and at least a portion of its associated components may be included in each of the mask “openings”, including but not limited to air inlets 3102, oral opening 3104, and air/oxygen input 3106 which is used to attach air or oxygen tubing. In embodiments, one or more of the filter housing 3113 and/or the filter caps 3111 are designed to accept quick-connect port 3123 to connect tubing for gas delivery or apply suction to the inside of the mask.

In embodiments, the replaceable/removable filter 3125 has a filter rating of N90 or greater as defined by US National Institute for Occupational Safety and Health (NIOSH) classification of air filtration, meaning that it filters at least 90% of airborne particles. In embodiments, the filter 3125 is used to filter out droplets greater than 0.1 μm. In some embodiments, the filter 3125 is used to filter out droplets greater than 0.3 μm. In embodiments, the filter 3125 is used to filter out droplets greater than 0.5 μm. In embodiments, the filter 3125 thickness ranges from 0.1 to 1.5 mm. In embodiments, the filter 3125 has a thickness of less than 5 mm. In embodiments, the thickness of filter 3125 ranges from 1 to 1.5 mm. In embodiments, a filter material is employed to cover greater than or equal to 10 percent and less than or equal to 95 percent of the mask body 3101. In some embodiments, the filter 3125 is an electrostatic filter and made out of electrically charged material.

FIG. 31D is a perspective view of the mask shown in FIG. 31A. As shown in FIG. 31D, mask 3100 includes air inlets 3102, an air/oxygen input 3106 with an optional tubing connector 3121, and an endoscope port 3104. Air/oxygen input 3106 is also fitted with a filter housing (such as housing 3113 of FIG. 31C) as its associated components, including a filter like filter 3125, as described above. FIG. 31E is a side elevation view of the mask 3100 shown in FIG. 31D. FIG. 31F is a rear elevation view of the mask 3100 shown in FIG. 31D. As seen in FIG. 31F, mask 3100 includes air inlets 3102, air/oxygen inlet input 3106, and an endoscope port 3104. The air/oxygen input 3106 includes an inlet valve 3107 and is used for attaching oxygen or air tubing either directly or via an optional tubing connector or port 3121. In various embodiments, any of the inputs/ports/openings/or access points of the mask can be adapted to provide any of the functions such as the use of instrumentation during a medical procedure, gas delivery, and/or suction.

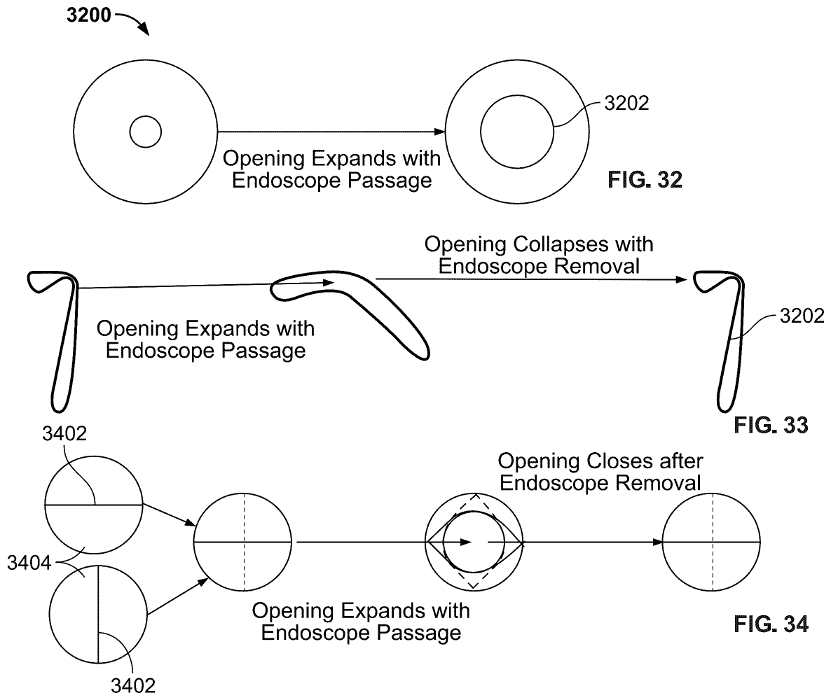

FIG. 31G is a perspective view of the mask 3100, showing a low-profile oral endoscope port 3104 in accordance with an embodiment of the present specification. In an embodiment, the endoscope 3104 port has a low profile in order to incorporate a removable sleeve, as described above with reference to FIG. 31C and below with reference to FIG. 32. By way of comparison, FIG. 31H is a side elevation view of a mask 3100 in accordance with an embodiment of the present specification showing a high-profile oral endoscope port 3104h, while FIG. 311 is a side elevation view of a mask 3100 in accordance with a preferred embodiment of the present specification showing a low-profile oral endoscope port 3104i. FIG. 31H shows the position of port 3100h at the most distant surface on the curved surface of the mask, such that in an exemplary embodiment, a horizontal span of the mask from the inflatable rim to the oral endoscope port 3104h is approximately 59.69 mm. Whereas in FIG. 311, the horizontal span of the mask, viewed from the side elevation of the mask, is approximately 43.57 mm. In some embodiments, the port 3100i is at the same horizontal level as the air inlet ports on the mask.

FIG. 31J is a bottom perspective view of a mask in accordance an embodiment of the present specification having an oral port 3104j, wherein the oral port is preferably low profile. In addition, it is preferred that the oral port is flat or flush with the mask and not protruding. In other embodiments, it is also preferred that the other openings, such as the air/oxygen input 3106 are flush with the mask or not protruding. For example, instead of using a tubing connector or port 3121, it is possible (and preferred) to screw an oxygen tubing around the filter portion of inlet valve 3107 (where oxygen is delivered directly through the filter). In other embodiments, a low-profile and/or flat quick connect mechanism may be employed. In embodiments, the mask is shaped to conform to a human face such that the oral port/opening/input 3104 is positioned closer to the oral cavity for improved use of instrumentation.

FIG. 31K is a bottom perspective view of a mask 3100 in accordance with an embodiment of the present specification, with a quick connect port (3121 of FIG. 31J) removed. FIG. 31L is an exploded, perspective view of the quick connect-to-tubing mechanism shown in FIG. 31J. As shown in FIG. 31L, the air/oxygen input 3106 includes a tubing connector or port 3121, a valve 3107 that includes a thread fixing hole 3115, and a filter 3117. In embodiments, any suitable filter may be selected. In embodiments, the filter is made of cotton, polytetrafluoroethylene (PTFE), expanded polytetrafluoroethylene (ePTFE), glass fiber, nylon, or any other filter material known in the art.

FIG. 31M is a side perspective view of the quick connect mechanism shown in FIG. 31L, connected to a hose/tubing. As shown in FIG. 31M, an oxygen port 3119 is connected to a hose/tubing 3114 via the air/oxygen input port 3106, through a threaded hole 3115. FIG. 31N is a top front perspective view of the air/oxygen input port 3106, where an oxygen connector port 3119 is threaded into the thread fixing hole 3115 shown in FIG. 31L in lieu of tubing connector/quick connect port 3121.

FIG. 31O shows a first perspective view 3140 and a second close-up view 3142 of an actual photograph of the mask shown in FIG. 31A that is configured for application of suction and also configured to receive an optional droplet control sleeve, in accordance with some embodiments of the present specification. As shown in the views 3140, 3142 at least one of the air inlets 3102 includes a tubing connector/quick connect port 3145 for connection to a hosing or tubing through which suction may be applied to the port 3145 in order to remove droplets created during various respiratory activities via suction. View 3140 shows a droplet control sleeve 3109 removed from the oral opening or endoscopic port 3104 while view 3142 shows the droplet control sleeve 3109 fitted into the oral opening or endoscopic port 3104. View 3140 also shows the air/oxygen input 3106 with the tubing connector/quick connect port 3121.

In some embodiments, the mask provides a soft nasal bridge adjustment 3180 which allows the length of the mask from the chin and the nose to be adjusted to accommodate the variable length depending upon whether the patient's mouth is open or closed during a procedure. Also, the mask is shaped according to the contoured surface of the nose so that the mask does not slip up on the nose bridge, accidently irritating/damaging the eyes or the cornea.

FIG. 31P illustrates first and second views 3160, 3170 showing the connector/quick connect port 3145 connected to a first tubing 3162 that further connects to a second tubing 3164 and a third tubing 3165 via a T connect 3163. Suction is applied at a proximal end of the second tubing 3164 while a proximal end of the third tubing 3165 leads to a sensor for carbon-dioxide monitoring. As shown in the first view 3160, a Yankauer suction tip 3167 is in fluidic connection with the connector/quick connect port 3145 through a fourth tubing 3169. Application of suction at the proximal end of the second tubing 3164 causes suction of oropharyngeal secretions of a patient using the mask. View 3170 shows a fifth tubing 3172 connected to the tubing connector/quick connect port 3121 of the air/oxygen input 3106.

In various embodiments, the mask is configured to enable performing a procedure through the mouth or nose of a patient, wherein the mask includes an oral opening or endoscopic port to pass a medical instrument and a suction port. In some embodiments, application of suction, through the suction port, reduces the number of respiratory droplets released during the procedure into the room air by at least 10%.

FIG. 31Q illustrates a soft nasal bridge adjustment 3112q that is provided on a top portion of a seal 3110q for a procedural mask 3100q, in accordance with some embodiments of the present specification. A first view 3102q shows the side elevation of mask 3100q, and a view 3104q shows the front view of the mask 3100q. The adjustment 3112q allows a length of the mask from the chin and the nose to be adjusted to accommodate the variable facial length depending upon whether the wearer's mouth is open or closed during a procedure. In some embodiments, a curved steel clip 3114q is provided on an external surface of bridge adjustment 3112q to further adjust by tightening or loosening the grip of bridge adjustment 3112q over the nose. To ensure a good fit with mouth open, chin portion of the seal pulls mask down, as it is configured like an accordion that allows for an extra length on the vertical movement so that a user can open and close the mouth and the seal will still cover their nose. Additionally, in some embodiments, the upper portion of the mask body is shaped according to shape of a nose so that the mask does not slip up on the nose bridge, accidently irritating/damaging the eyes or the cornea.

FIG. 31R illustrates an exploded view of a procedural mask 3100r assembled with various components, in accordance with some embodiments of the present specification. FIG. 31S is a flow chart illustrating exemplary process of using the procedural mask 3100r, in accordance with some embodiments of the present specification. Referring simultaneously to FIGS. 31R and 31S, the mask 3100r is preferably placed over a wearer's face, such as a patient, centering on both the nose and the mouth. At step 3102s, an elastic strap (not shown) attached to the mask 3100r, is placed below the ear and around the back of the head. Ends of the strap may be pulled and adjusted for comfort and fit. At step 3104s, prior to a procedure, an oxygen tubing connector 13 is attached to one of the tubing connections on the filter ports (left, right or bottom ports as needed), and secured tightly. Tubing connector 13 is attached to an oxygen source. At step 3106s, the oxygen is set to the desired flow and oxygen flow through the mask is checked. At step 3108s, a suction tubing 14 is attached to one of the tubing connections 13 on the filter ports (left, right or bottom ports as needed), and secured tightly. Tubing 14 is attached to a suction source. At step 3110s, once all connections are secure, gas flow and suction are started. At step 3112s, an instrumentation sleeve 4 is attached after removing an opening cover 1 from the main body of mask 3100r by unscrewing counterclockwise. In embodiments, the cover 1 is replaced with instrumentation sleeve 4 which is then secured in place. The cover 1 may be saved for use after the procedure. At step 3114s, the procedure is performed. During a procedure, an instrument is inserted in the opening through the instrumentation sleeve 4. At step 3116s, upon completion of the procedure, the suction tubing 14 and instrumentation sleeve 4 is removed from the mask 3100r. At step 3118s, the oral opening is closed with an air filter 8 cover. The oxygen tubing connector 13 is disconnected from mask 3100r in the recovery prior to discharge.

FIG. 32 illustrates an embodiment of an oral opening/valve 3200 shown as opening/valve 3104 in FIG. 31A. In an embodiment, the oral opening/valve 3200 is a diaphragm membrane with an opening 3202 ranging from 2 mm and expanding up to 22 mm to fit snugly around the shaft of an endoscope. Thus, the opening 3202 expands with the passage of an endoscope. A flap valve is used to cover the opening so that there are no aerosol leaks when the endoscope is removed or not present.

FIG. 33 illustrates another embodiment of the oral opening/valve shown in FIG. 31A. In this embodiment, the oral opening includes a sleeve 3302 that fits tightly around the endoscope during insertion yet closes itself completely when there is no endoscope in place.

FIG. 34 illustrates yet another embodiment of the oral opening/valve shown in FIG. 31A. Oral opening/valve 3400 includes slits 3402 in two membranes 3404 which are perpendicular to each other completely closing the oral opening when there is no endoscope inserted.

FIG. 35 illustrates two exemplary air inlets that may be used with embodiments of the present specification as described with respect to FIGS. 30A/30B and 31A. The air inlet embodiments may have replaceable antimicrobial or particulate filters. Air inlet 3500a has a circular form, while air inlet 3500b has a square configuration. In embodiments, the air inlet has any shape that may be configured on the sides of the mask.

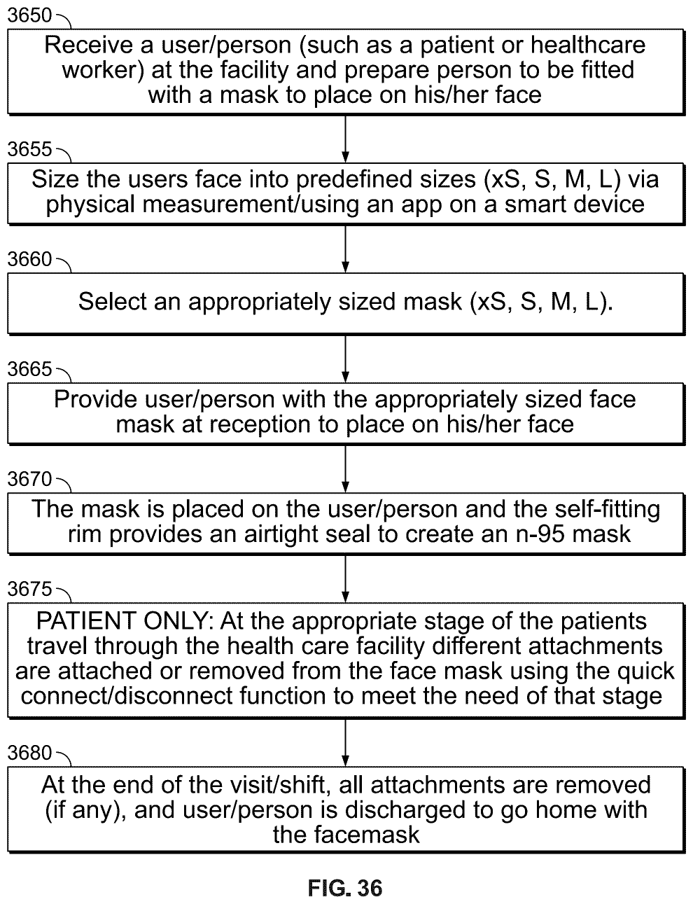

FIG. 36 is a flow chart that describes the steps involved in using a procedural/healthcare worker mask in accordance with the present specification. When a user/person/patient arrives at or checks into a hospital, clinic, surgery center or other sterile setting, at step 3650, they are prepared to be fitted with at least one mask described in embodiments of the present specification. In embodiments, the user's face is fitted with a mask of a predefined size, in step 3655, via a physical measurement or using an app on a smart device. In embodiments, the predefined sizes may include sizes such as extra small, small, medium, large or extra-large, or any other type of size categorization. In embodiments, the smart device may include a smart phone, a computing device, or any other device that is suitable for the purpose. In embodiments, the smart device employs facial recognition software. In step 3660, an appropriately sized mask is selected based on the measurement. In step 3665, an appropriately sized mask is provided to the user based on the selection. In step 3670, the mask is placed on the user/patient/healthcare worker, whereby the self-fitting rim provides an airtight seal to create an N-90 or an N-95 mask. The user will wear this mask throughout their shift (in the case of a healthcare worker) or encounter (in the case of a patient), thus preventing passage of aerosolized pathogens to and from the user to patients or healthcare workers or anyone present at the facility. In the case of use with a patient, it should be noted that prior to the procedure, the patient can freely ambulate without oxygen tubing. A plurality of the ports on the front of the mask direct the air inward and outward through the filters while trapping the respiratory droplets. In the case of use with a patient, as the patient travels through a healthcare facility, in step 3675, different components are attached or removed from the face mask using the quick connect/disconnect mechanisms, thus meeting the need of that particular stage of patient care. For example, the detachable oxygen tubing may be attached when the patient is ready for their procedure. In addition, the mask is fitted with an oral opening with a one-way valve for passage of an endoscope where the valve fits tightly around the endoscope, preventing leakage of any aerosolized oropharyngeal secretions from the patient. The oxygen tubing enters the mask below the oral opening so as not to interfere with insertion of the endoscope. On conclusion of the procedure, at step 3680, the oxygen tubing is detached, and patient is discharged home with the mask. In the case of a healthcare worker, at step 3680, at the end of the shift, the healthcare worker is free to go home with the mask, thus ensuring minimal pathogen exposure throughout their shift. In this manner, the pathogen exposure to the user and from the user from the entrance of the hospital/surgery center/facility/retirement home throughout the user's stay is minimized.

Multiple embodiments of a procedural mask can be configured in accordance with the present specification, as used in the method described in FIG. 36A. In some embodiments, the mask includes an oral opening and may include optional air filters. In embodiments, the oral opening ranges in diameter from 10 mm to 30 mm, covering a surface area in a range of 75 square mm to 700 square mm. A detachable oxygen tubing is attached to mask at the beginning of a procedure. An optional, detachable rebreather bag is also attached to mask at the beginning of a procedure, wherein the bag has a volumetric capacity ranging from 100 cc to 500 cc. The rebreather bag serves to both increase FiO2 and trap aerosol during retching, coughing, or sneezing. The mask may be configured without optional air filters. An oral tool is optionally inserted through the oral opening of mask. In embodiments, the oral tool is an endoscope. An oral valve (which is fitted with the oral opening) fits snugly around the shaft of an endoscope so as to minimize passage of aerosol through the oral opening while the oral tool or instrument is inserted. Further, mask is fitted with at least one flap valve covering at least one side port to minimize or prevent passage of aerosol. The oral valve collapses like a sleeve to minimize or prevent passage of aerosol through the oral opening while the oral tool is not present or is withdrawn. An additional opening may be configured in the mask, which is similar to the oral opening, to pass additional instrumentation through.

A mask as used in some embodiments of the present specification, has a mask body with various filter inputs/ports/openings. Tubing connectors/quick connector ports are positioned within filter housings that include associated filter components. In embodiments, the mask has a horizontal width spanning from a left side of the mask to a right side, of approximately 115 mm. In various embodiments, the top portion of the mask rests on the lower bridge of the nose, substantially proximate to the junction of the cartilaginous and bony part of the nose allowing for the upper nasal bridge to be available for wearing eyewear. In various embodiments, the bottom of the mask rests under the chin, preventing the mask from sliding upwards with jaw movements. The soft, flexible compressible rim allows for a tight, uniform, and comfortable fit around the entire circumference of the mask. The flexibility of the seal ensures that a perimeter of the transparent surface is positioned on the skin such that no airflow is permitted to pass across the sealed perimeter.

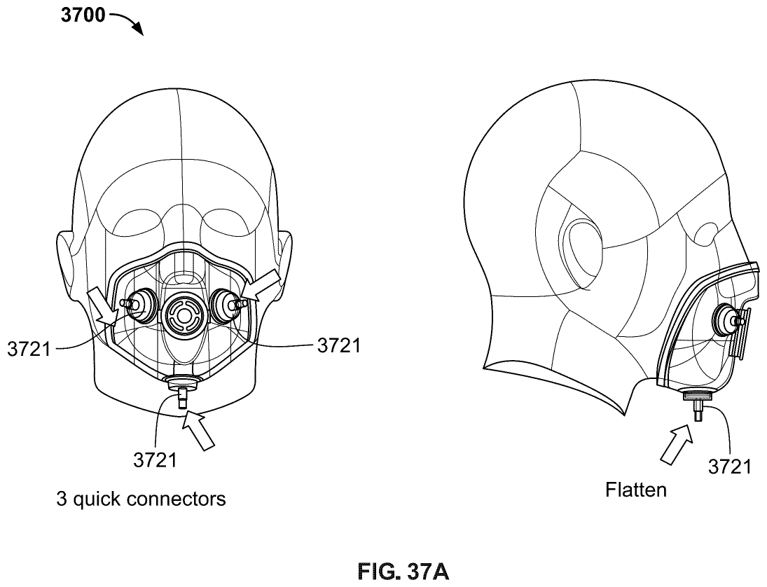

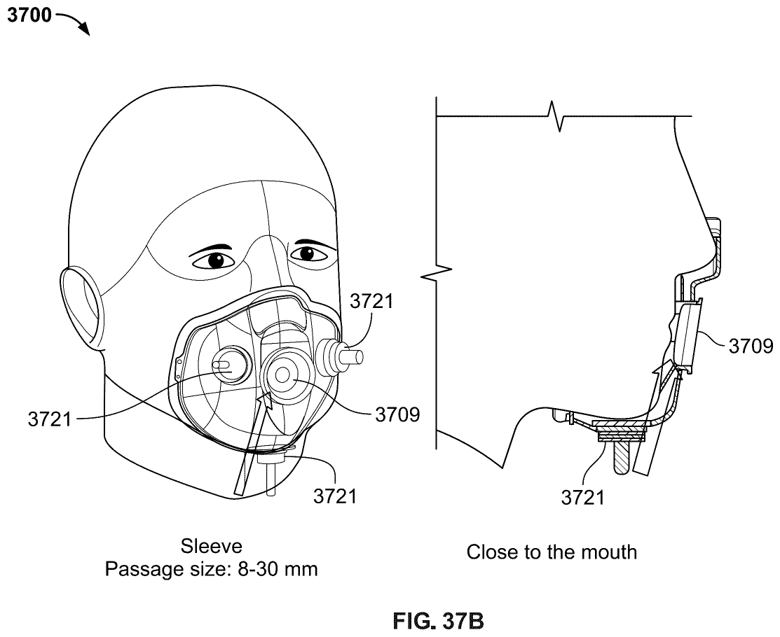

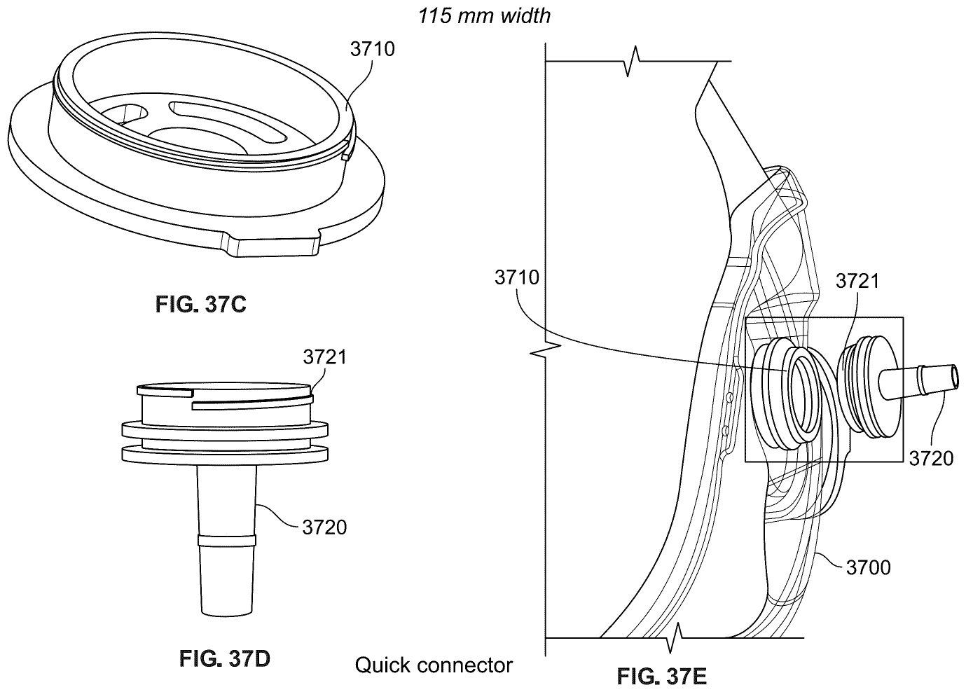

FIG. 37A shows front and side views of a person wearing a mask in accordance with some embodiments of the present specification, where the mask includes three tubing connectors/quick connection ports 3721 located within filter housings for connection to a housing or tubing for clinical uses such as oxygen delivery or suction. It should be noted herein that the descriptions with respect to particular uses of any of the openings/ports/inputs described herein are meant to be exemplary and not limiting. Thus, all openings/ports/inputs can be used interchangeably for instrumentation during various medical/surgical procedures.

FIG. 37B shows front and side views of a person wearing a mask 3700, in accordance with some embodiments of the present specification. The mask 3700 includes a sleeve 3709 positioned within an oral/instrument valve opening. In embodiments, the sleeve 3709 allows for the passage of an instrument or device having a diameter ranging from 8 mm to 30 mm. The mask is shaped to approximate the facial contour of a human placing the oral port close to the mouth/oral cavity for easy instrumentation.

FIG. 37C is a perspective view of a filter housing 3710. FIG. 37D is a perspective view of a quick connect port 3720 with threading 3721. FIG. 37E is a side-rear view of a person wearing a mask 3700 in accordance with some embodiments of the present specification, where the mask includes a quick connect port 3720 with a threaded connection 3721 to a filter housing 3710 as shown in FIGS. 37C and 37D. Any quick-connect mechanism known in FIGS. 37C, 37D, 37E can be used to connect a multitude of detachable connectors that may be provided with the mask.

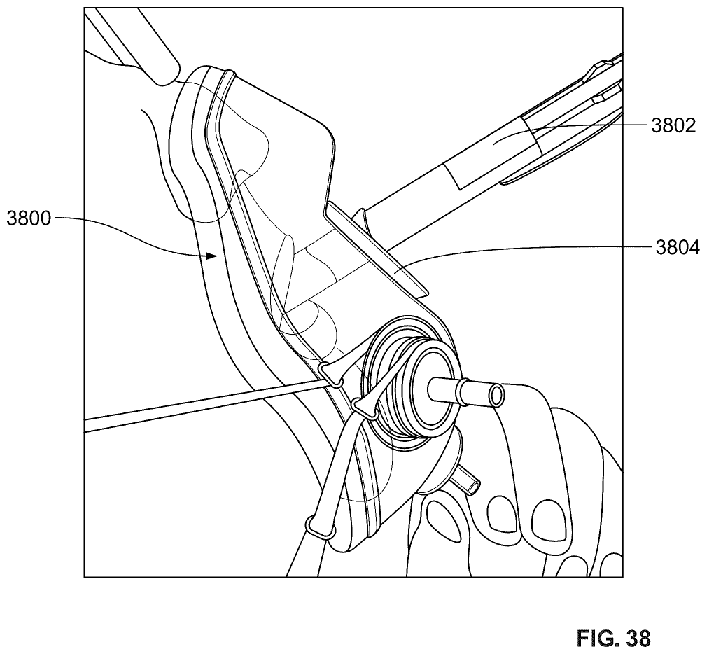

As described previously, the multiple embodiments of masks of the present specification are configured in different sizes to fit persons of different face or head sizes. In some embodiments, the masks are configured with a width of 115 mm. In some other embodiments, the masks are configured with a width of 85 mm. FIG. 38 illustrates an exemplary embodiment of an 85 mm mask worn by a user, with a tool 3802 inserted through an oral opening 3804 of the mask 3800, in accordance with some embodiments.

### MASK STERILIZER

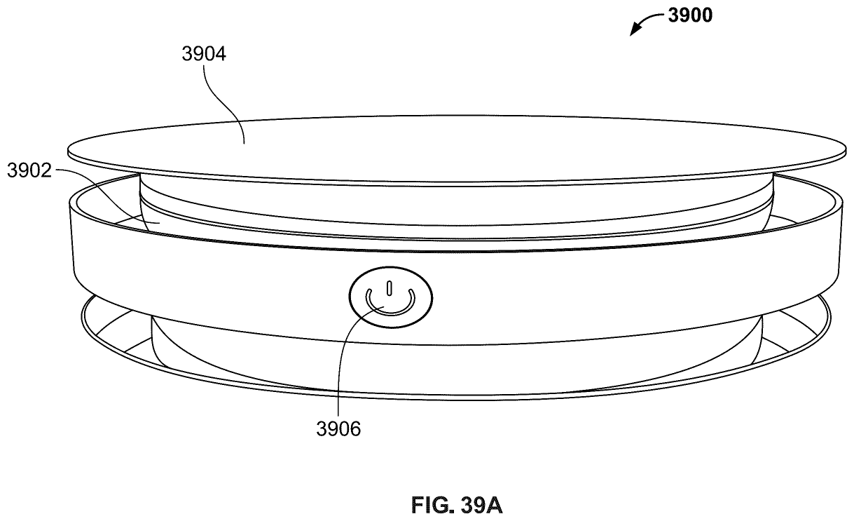

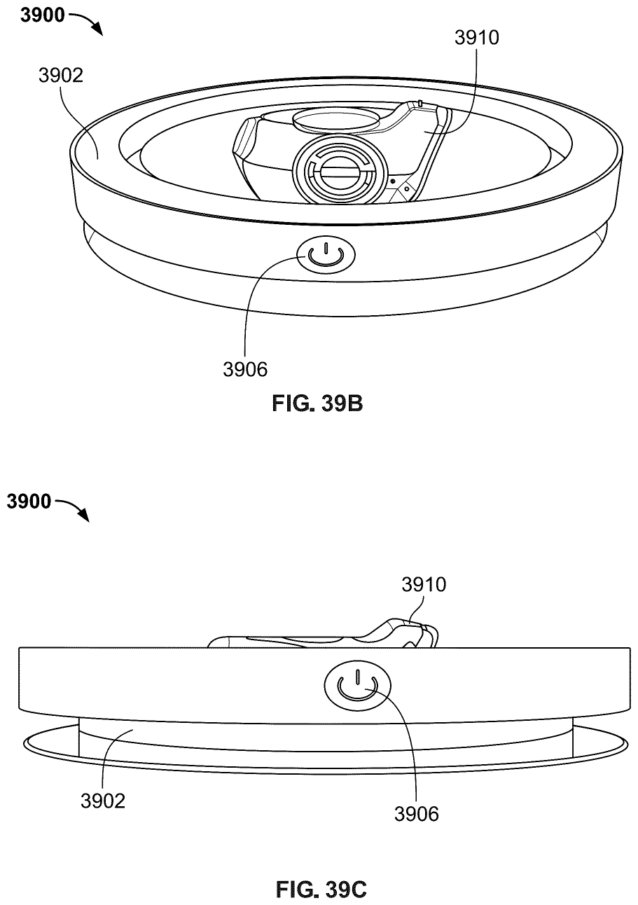

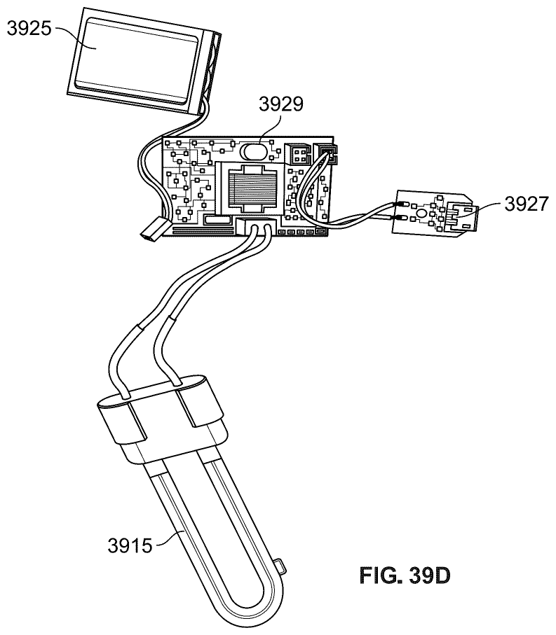

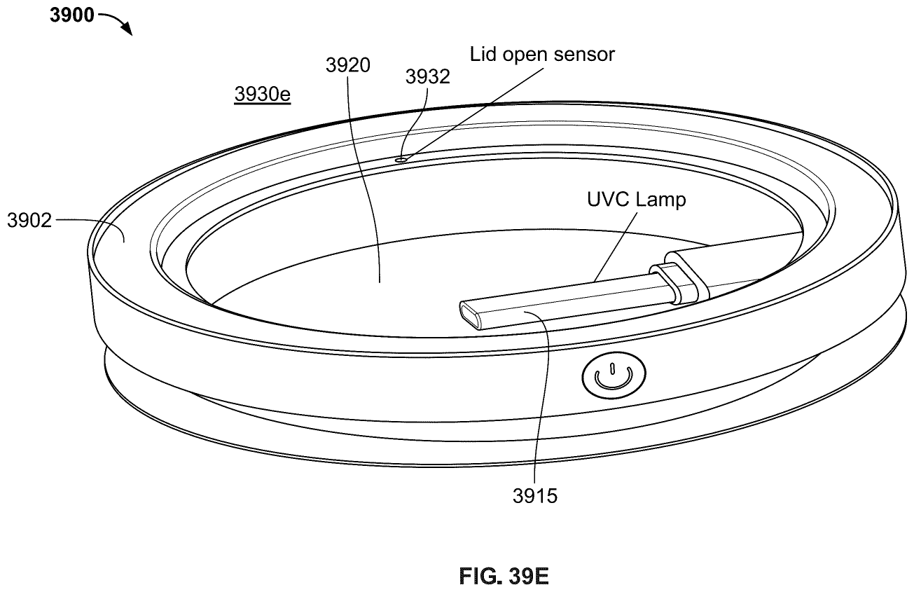

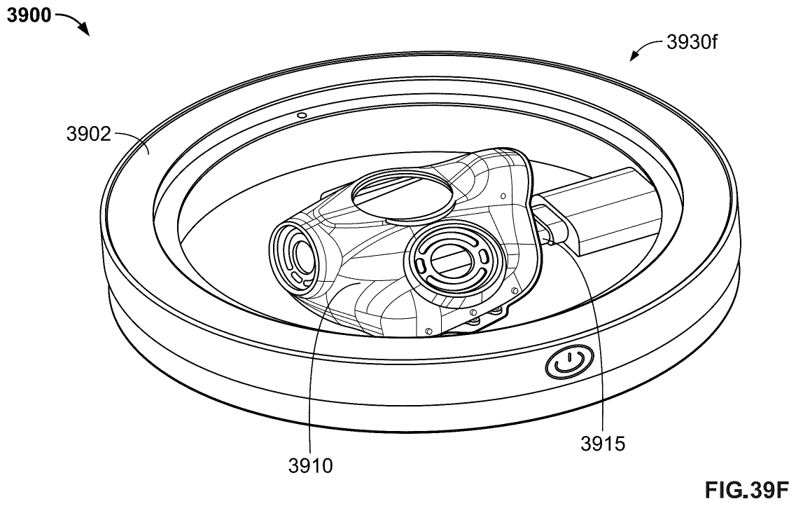

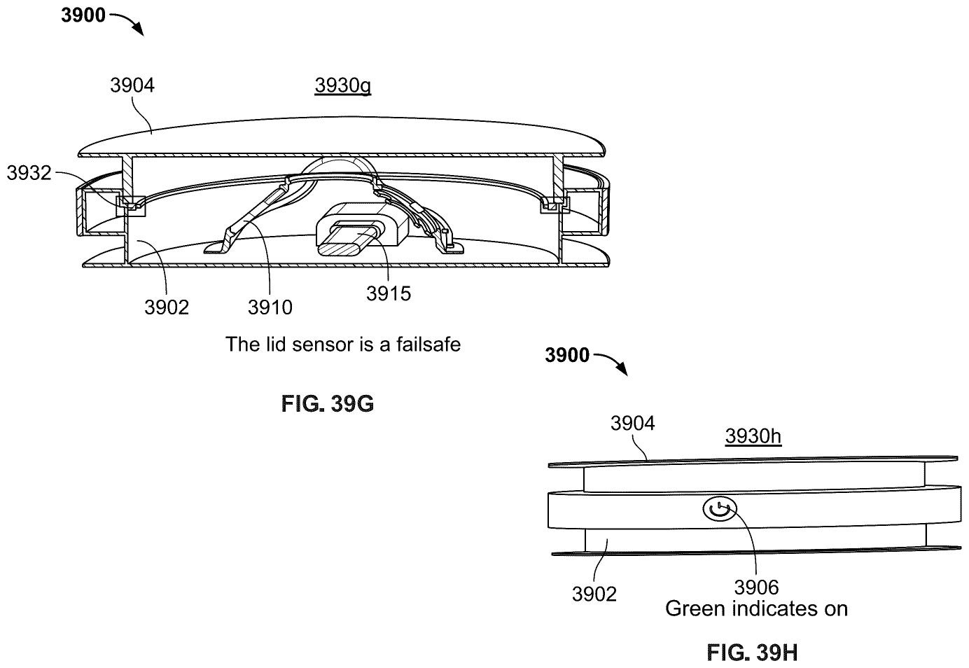

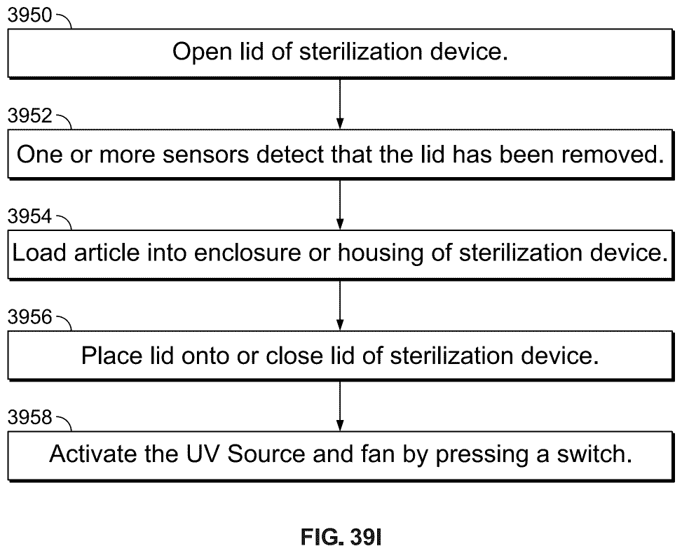

FIG. 39A shows a perspective view of an Ultra-Violet C (UV-C) sterilizer device 3900, in accordance with some embodiments of the present specification. The device 3900 has an enclosure or housing 3902, a removable lid 3904 and a switch 3906 to switch on or off the device 3900. FIGS. 39B and 39C show first and second side views of the device 3900 with the lid 3904 removed and an article 3910 positioned within the housing 3902 for sterilization.

In various embodiments, the internal surfaces of the enclosure or housing 3902 have a reflective coating. As shown in FIGS. 39E, 39F and 39G, the enclosure or housing 3902 includes a UV-C light source 3915, a first surface 3920 coated with TiO₂ and exposed to the UV source 3915, an activated charcoal filter and a fan to circulate air. In embodiments, the fan is positioned anywhere to create an air circulation. Air is circulated through the filters in the mask and in and around the mask. The flow of the air is also routed through the filter which, in embodiments, is located at the intake or the outflow of the fan. The activated charcoal filter is configured to absorb harmful chemicals, for example, formaldehyde (HCHO) and volatile organic compounds (VOCs) which could be generated by photocatalytic oxidation (PCO), ionization, or by breakage of organic material in the mask. For sterilization, an article 3910 such as, for example, a mask of the present specification is positioned within the enclosure or housing 3902 so that the article 3910 sits between the TiO₂ surface 3920 and activated charcoal filter. The fan circulates the ions generated from the TiO₂ surface 3920 along the surface of the article 3910. The UV source 3915 sterilizes the article 3910.

FIG. 39D shows a lithium ion battery 3925, a micro USB connector 3927 and the UV lamp 3915 of the sterilizer device 3900 attached to a printed circuit board 3929.

Referring now to FIG. 391, with reference to FIGS. 39E through 39H, in order to sterilize the article 3910, as shown in 3930e, at step 3950, the lid 3904 (FIG. 39A) of the device 3900 is opened. At step 3952, one or more sensors 3932 detect that the lid 3904 has been removed. At step 3954, and as shown as 3930f, the article 3910 is loaded in the enclosure or housing 3902. At step 3956, and as shown as 3930g, the lid 3904 is placed in order to close the enclosure or housing 3902. At step 3958, and as shown as 3920h, the switch 3906 is actuated (such as by pressing the switch for a few seconds) causing the UV source 3915 and the fan to be activated.

### NASAL PRONGS

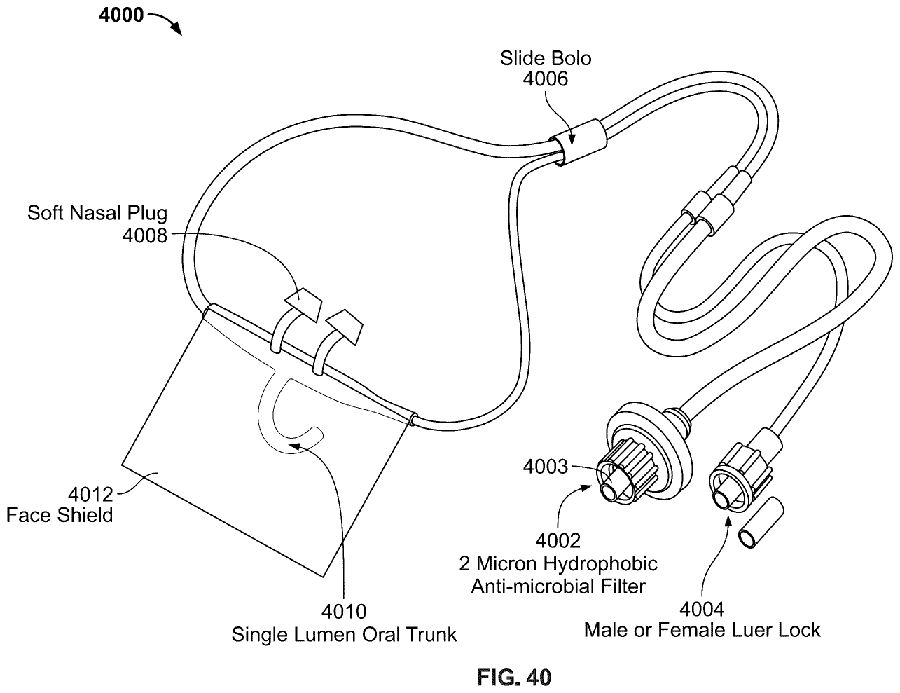

FIG. 40 is an illustration of an exemplary personal air delivery device that may be used in embodiments of the present specification. Personal air delivery device 4000 may include, in embodiments, a hydrophobic anti-microbial filter 4002 fitted with port 4003 and a port 4004. In embodiments, ports 4003 and 4004 include a male or female luer lock. In embodiments, either one or both ports 4003 and/or 4004 may be used for connection to an air pump (not shown). In embodiments, a second air pump may be fitted to increase air flow. In embodiments, personal air delivery device 4000 may also include a slide bolo 4006 for proper fitting onto a person, a soft nasal plug 4008; a single lumen oral trunk 4010; and a face shield 4012. The face shield provides, in embodiments, additional mechanical protection in addition to the protection provided by the air shield. A face shield may be ideal for use in high-risk areas, such as hospitals or meat-packing plants. The face shield also concentrates the flow of filtered air around the naso-oral area such that the air flows outwards around the face shield preventing infected air from entering behind the air shield.

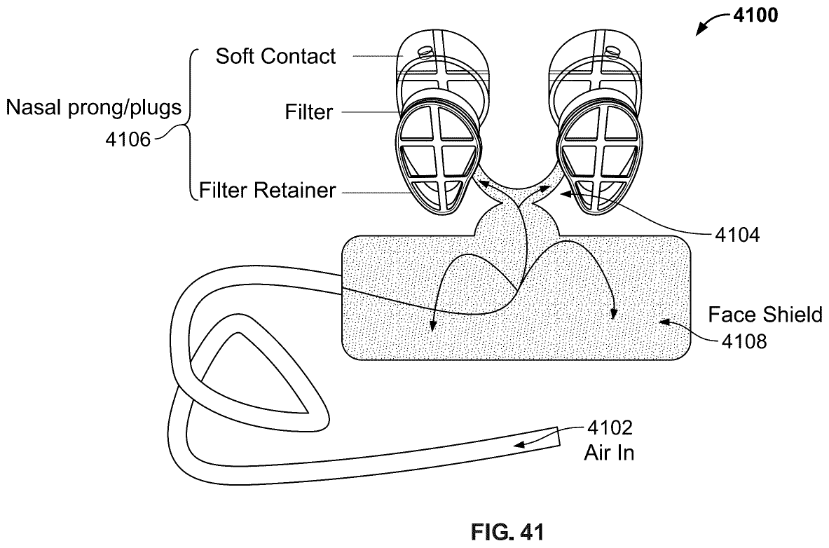

FIG. 41 is an illustration of a low-profile filtered air delivery system 4100 and integrated shield. System 4100 includes a tubing 4102 for receiving air at one end and for connection to a personal air delivery device 4104 at the other end. The personal air delivery device 4104 includes nasal prong/plugs 4106, which further includes a soft contact, filter, and filter retainer. In addition, personal air delivery device 4104 includes a face shield 4108. In this embodiment, the filtered air is partially delivered to a patient's nasal passages preventing unfiltered air from entering the nasal passages. In addition, the nasal plugs 4106 with integrated filters are used to filter any unfiltered air that enters the nasal passages. Majority of the air enters through the face shield 4108 to create an air shield around the mouth and face of the person, mechanically dislocating, circumventing, or deflecting infected or unfiltered air away from the face and mouth of the person.

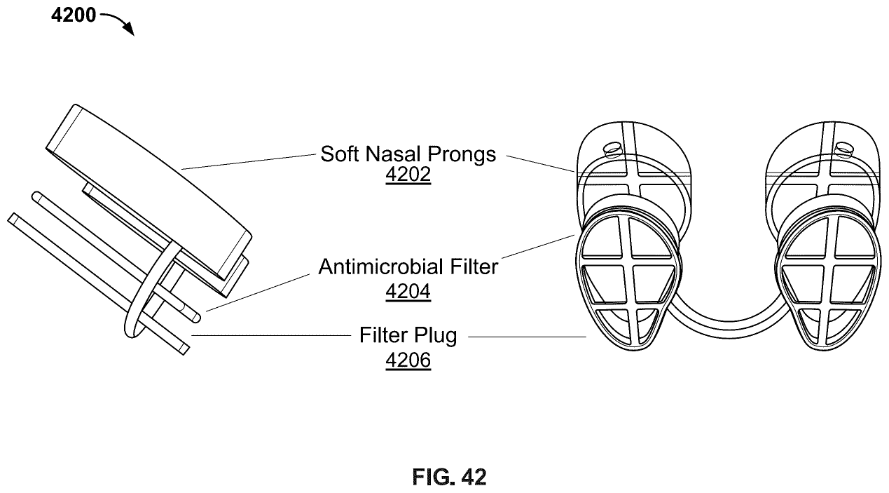

FIG. 42 is an illustration showing a nasal prong filter 4200 (nasal plug 4106 of FIG. 41) that may be used in embodiments of the present specification. The nasal filter 4200 includes a soft prong 4202 for comfort and long-term wear in the nares, a removable/disposable antimicrobial filter 4204, and a filter plug 4206 to hold the filter in place within the nasal prongs 4202.

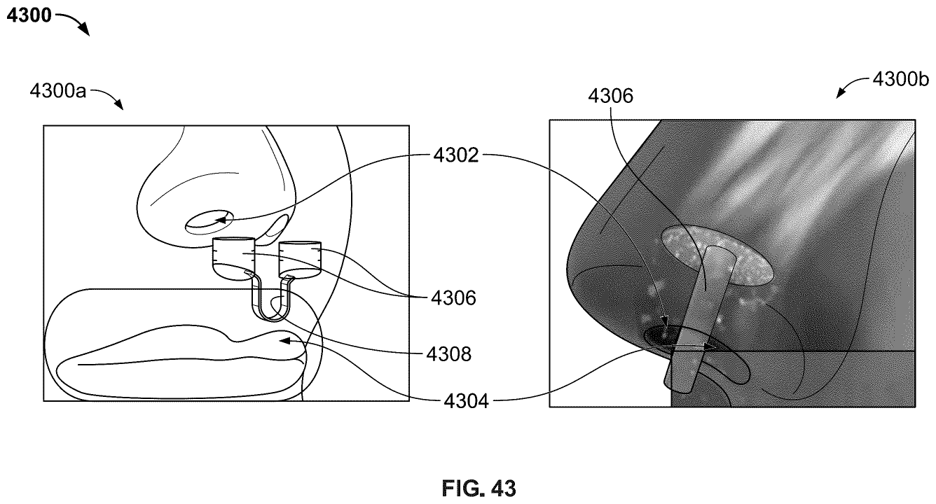

FIG. 43 is an illustration showing another embodiment of a nasal prong filter 4300 that may be used with the present specification. The figure also illustrates the filter 4300 as worn by a user in nares 4302 with a face shield 4304 covering the mouth of the user. A first view 4300a shows the position of filter 4300 proximal to and outside the nares 4302. A second view 4300b shows a transparent side view of the filter 4300 that has entered through nares 4302 inside the nose. In embodiments, the filter includes a pair of prongs 4306 that are cylindrical in shape so that they can glide through the nares into the chambers of the nose. The pair of prongs 4306 are positioned adjacent to each other, with a small gap, and are connected at the bottom with a bridge 4308. The bridge 4308 is a tube that further connects to a source of filtered air, such as an air filtration and pump configuration. Filtered air is delivered through the nasal prongs 4306 and through the face shield 4304 in and around the mouth, creating an air shield. A percentage of the filtered air is used by the person for breathing while majority of the air flows around the naso-oral region, creating the protective air shield.

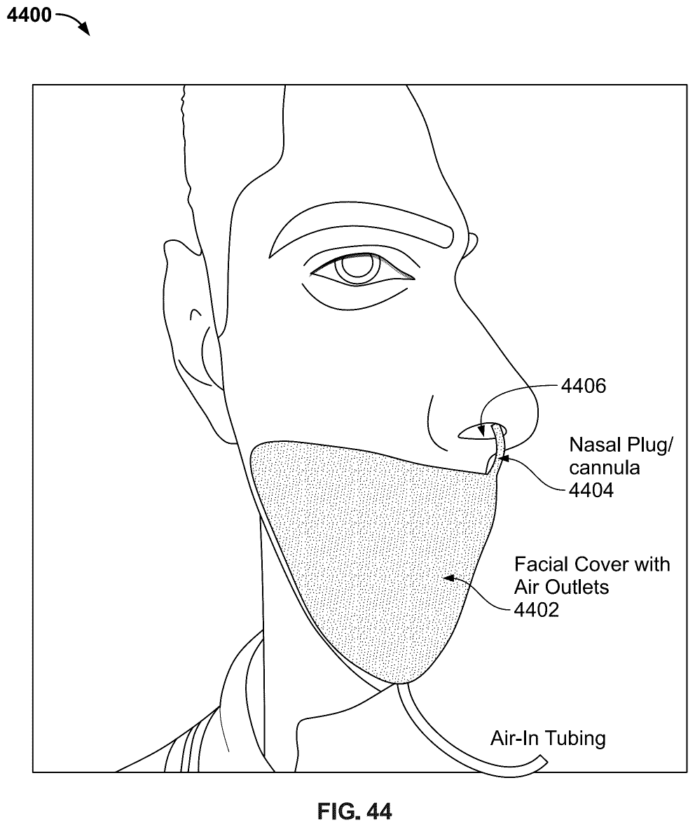

FIG. 44 is an illustration of a low-profile filtered air delivery system 4400 and integrated facial cover 4402 as worn by a user/patient. The two nasal prongs 4404 deliver a portion of the filtered air into the patient's nares 4406 for breathing and also for displacing/replacing any unfiltered air around the nares 4406. The two nasal prongs 4404 deliver a portion of the filtered air into the patient's nares 4406 for breathing and displacing or replacing any unfiltered air around the nares 4406. A percentage of the filtered air is used by the person for breathing while majority of the air flows around the naso-oral region, creating the protective air shield.

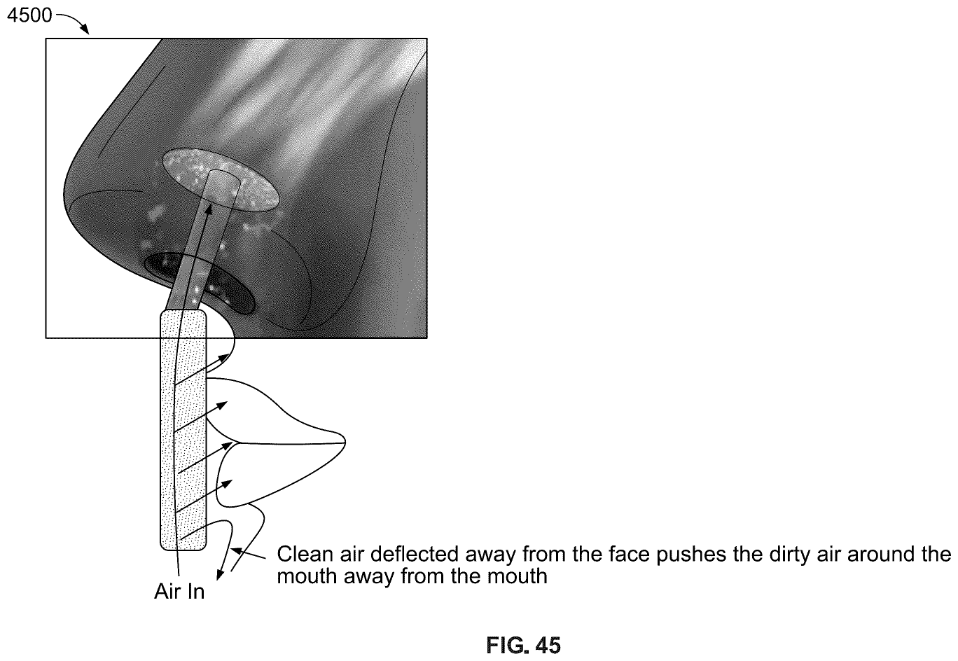

FIG. 45 is an illustration of the operation and air flow of a low-profile filtered air delivery system 4500, when worn by a user. Filtered air is provided to the naso-oral area with a pump (not shown). The filtered air delivered by the pump, and not inhaled by the user, deflects away from the face of the user, and displaces the air in and around the facial area away from the face. The outward flow of air in and around the face forces the pathogens away from the naso-oral area compared to a normal breathing situation where the contaminated air flows toward the naso-oral area and pathogens enter a patient's lungs. In addition, clean air is deflected away from the face and pushes the dirty air that may be around the user's mouth to away from the user's mouth as described with respect to FIG. 6.

### AIR FILTRATION

Embodiments of an air filtration and management system can be implemented in the present specification. The air filtration and management system includes a battery-operated air pump with an air inlet, which, in embodiments, is fitted with an electrostatic filter. The system is also fitted with an inline filter that is connected to one end of an air tubing. The other end of air tubing is further connected to a personal air delivery device, such as a mask, at its other end. In embodiments, the flow of air is on the order of twice that of a user's respiratory requirement, such that the majority of the filtered air is used to create the airflow moving away from the person's naso-oral area resisting flow of infected air towards the person's naso-oral area.

An air pump includes, in embodiments, a battery, an air filter, a microbial filter, and a connection port for connecting an air tubing that is then connected to a personal air delivery device, such as a mask. In embodiments, the pump dimensions range from 1 inch to 4 inches. In embodiments, the battery power ranges from 3V to 24V, with a battery life ranging from 3 hours to 12 hours. In embodiments, the battery is rechargeable.

A particulate filter may be used with embodiments of the present specification. In embodiments, particulate filter 2400 is a hydrophilic PES, PTFE, glass microfiber membrane, or nylon filter that is housed in a polypropylene housing. In embodiments, various materials may be used for the housing. In embodiments, the particulate filter ranges in size from 5 mm to 100 mm. In embodiments, the pore sizes of the particulate filter ranges from 0.1 μm to 100 μm. In some embodiments, an electrostatic filter may be used due to its lower-resistance to airflow.

In embodiments, different types of antimicrobial filters may be used. In some embodiments, an antimicrobial filter is a hydrophobic PES, PTFE, glass microfiber membrane, or HEPA filter that is housed in a polypropylene or styrene housing. In embodiments, various materials may be used for the housing. In embodiments, the antimicrobial filter ranges in size from 5 mm to 100 mm. In embodiments, the pore size of the antimicrobial filter ranges from 0.1 μm to 100 μm.

In some embodiments, the air filtration system performs filtration using centrifugal, non-laminar air flow. The air filtration system operates at a noise level of less than 65 decibel during operation. Additionally, in embodiments, the rate of delivery of filtered air is greater than 30 L PM. Moreover, the air delivery ports that deliver the filtered air cover less than 95% of a surface of the wearable structure of the mask.

In some embodiments, a UV filter is employed and serves to kill airborne pathogens, and the filtration therefore performed is termed as destructive filtration. In some embodiments, a far Ultraviolet (UV-C) filter can be used with or without the mechanical antimicrobial filter. The use of a UV-C filter is advantageous as it has a very low resistance to airflow, improving the desired high flow rate. In embodiments of the present specification, the wavelength of the UV filter ranges from 207-280 nm. In embodiments, UV-C light is employed and ranges from 207 nm to 222 nm. In embodiments, the preferred wavelength is 222 nm. In embodiments, the UV-C light is sourced from either a UV-C LED or a UV-C lamp.

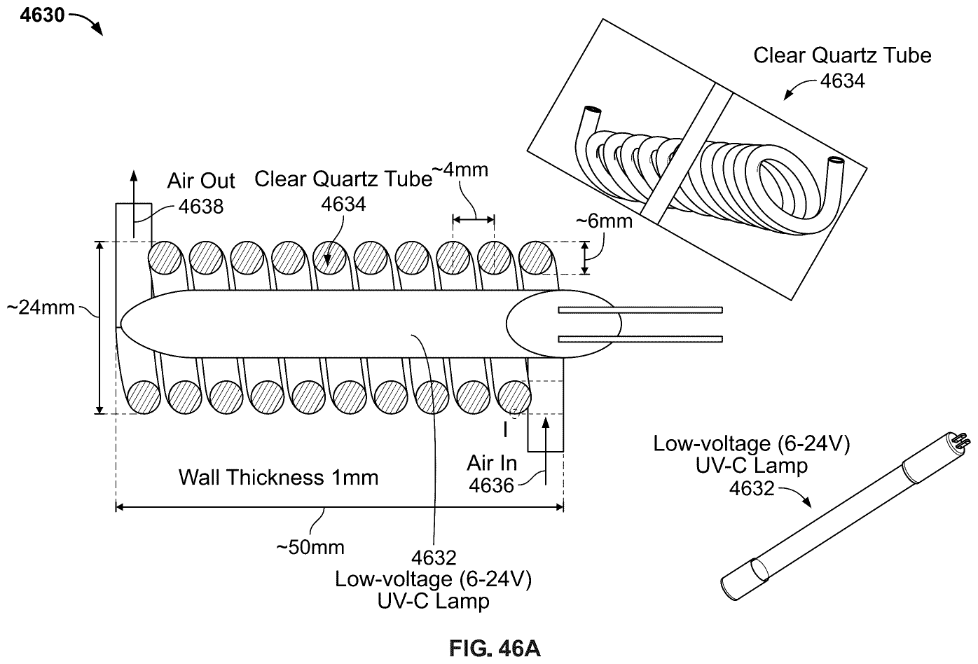

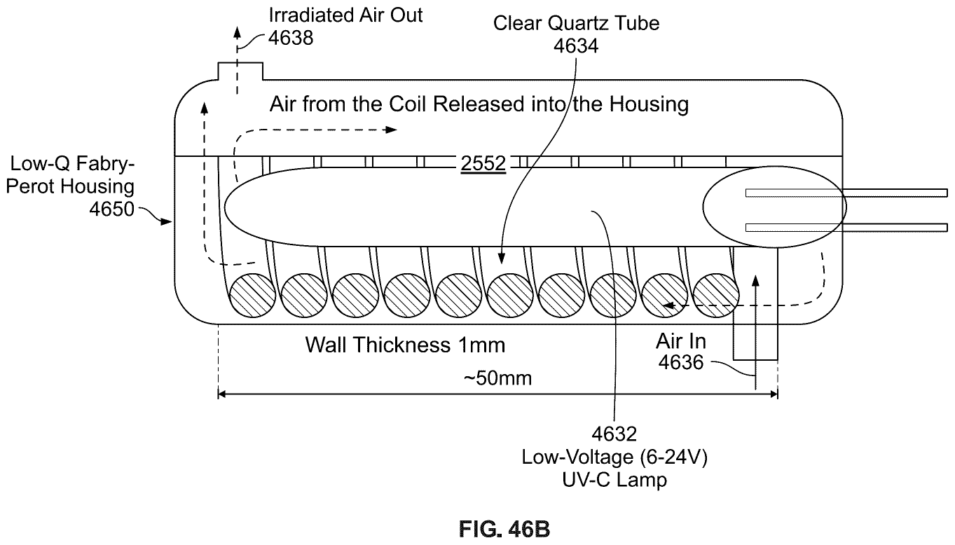

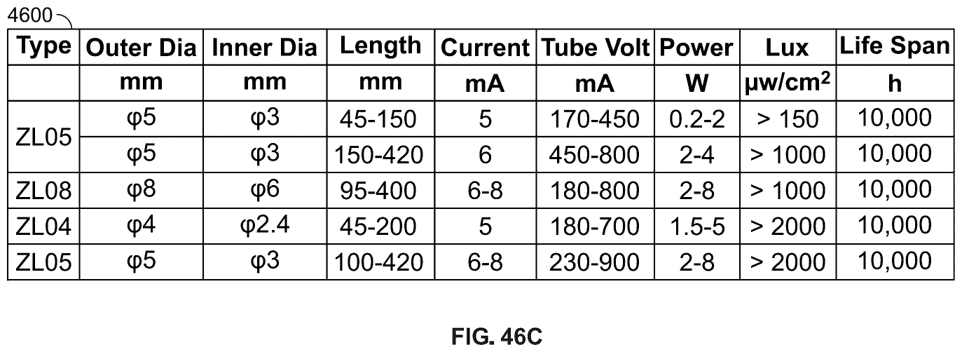

FIG. 46A is an illustration of an embodiment of a UV-C anti-microbial filter that may be used in conjunction with the embodiments described in the present specification. In an embodiment, the UV-C anti-microbial filter 4630 comprises a UV-C lamp 4632. In an embodiment, the UV-C lamp 4632 is of low voltage (ranging from 6V to 24V). The length of the lamp 4632 is in a range of 50 mm to 115 mm, and a width in a range of 5 to 15 mm. In embodiments, the UV-C lamp operates on the order of greater than 25 μW/cm², and ideally, greater than 100 μW/cm² (220 nm to 280 nm wavelength). The temperature of the lamp ranges from 25 degrees Celsius to 60 degrees Celsius. In embodiments, the exposure time of air to the lamp is greater than or equal to 1 second for optimal efficacy. A coil or tube 4634, which is fabricated from Ti doped clear quartz, Vycor, or high silica glass is coiled around UV-C lamp 4632. In some embodiments, a height of the UV-C lamp with the coil 4634 wound around it is approximately 24 mm. In some embodiments, the coil or tube 4634 has an inner diameter ranging from 3-4 mm, and is preferably 4 mm. The outer diameter of the coil or tube 4634 is approximately 4 to 6 mm. In embodiments, the coil or tube 4634 has a wall thickness of approximately 1 mm. It should be understood by those of ordinary skill in the art that any diameter or material of tubing may be used with the embodiments described herein as long as it achieves the necessary operational parameters. Coil or tube 4634 further includes an air inlet 4636 for receiving ambient, and possibly contaminated air, and an air outlet 4638 for outputting filtered, clean air. The use of a coil increases the path of air flow in a small space, thus increasing UV exposure time.

FIG. 46B is a schematic diagram of the filter shown in FIG. 46A encased in a housing. A low-Q Fabry-Perot cavity is created by coating the interior of the housing 4650 with a UV reflective material to increase the efficiency of the UV-C lamp 4632. Air is released from the coil 4634 into the housing 4650 to provide for additional circulation and contact time for air and UV-C rays outside the coil 4634 at the same time, thus cooling the UV-C lamp 4632 to an ambient operational temperature ranging from 30 degrees Celsius to 50 degrees Celsius, and preferably 40 degrees Celsius. In a plane 4652 that is defined by the source of the UV-C, when released into the housing or chamber, the air moves in a first direction orthogonal to the UV-C lamp 4632, a second direction planar to the UV-C lamp 4632, and a third direction orthogonal to the UV-C lamp 4632 while being irradiated by the UV-C lamp 4632. FIG. 46C shows various parameters of different types of UV-C sources 4600 that may be used with the embodiments described in the present specification.

A pump is assembled with various filters that may be used with embodiments of the present specification. A pump assembly may include a battery connected to a particulate filter, which is connected to a humidification chamber, which is further connected to an antimicrobial filter. Moreover, the antimicrobial filter is further connected to an air tubing for delivering filtered air through an air delivery loop, such as those used with nasal prongs. The air that flows through the nasal prongs and facial air ports create an air shield around a person's face and mouth.

In various embodiments, the PAFS and face masks described in the present specification are configured such that their total weight is less than 5 lbs and, more preferably, less than 1.33 lbs or approximately 600 grams. In embodiments, each PAFS individually and each face mask individually weighs less than 5 lbs and, more preferably, less than 1.33 lbs or approximately 600 grams.

Devices in the various embodiments of the present specification utilize air filtration mechanisms to purify circulated air to provide clean air to the user. The air is forced to pass through a HEPA filter for example, by a blower. In embodiments, the blower speed is regulated based on feedback from a user's breathing. In embodiments, the feedback mechanism may be mechanical or electromechanical. In one embodiment, the pressure changes in air due to a user's breathing rate, is measured by an onboard pressure sensor. The sensor signal is converted to an electrical signal that provides an input for adjusting the blower speed. The blower includes a fan mechanism that is operated with a motor. Heavy breathing increases the motor speed and therefore current consumption while slow breathing reduces the speed of the blower and the current consumption.

### ADVANTAGES

In accordance with various aspects, the masks described in the present specification are optically-clear and preferably comply with specifications of N95 masks or higher, that combine a comfortable clear elastomeric mask with a plurality of ports to house interchangeable replaceable filters, which serve both inhalation and exhalation functions and are optimized to minimize the resistance to airflow. The filters offer more than 25% reduction in aerosol inhalation over approximately 1 minute of sampling under test conditions. The optically-clear and flexible mask body yields more effective communication by allowing visualization of the wearer's face while also creating a secure seal around the perimeter of the mask and enabling it to be worn comfortably over long periods. The flexibility of the seal ensures that a perimeter of the transparent surface is positioned on the skin such that no airflow is permitted to pass across the sealed perimeter. In embodiments, greater than 4 square inches, which corresponds to approximately 25% to 90% of the surface area of the mask offers optical clarity for facial recognition.

Feasibility of the efficacy of the masks of the present specification has been established via bioaerosol-chamber ventilate manikin inhalation bioaerosol testing while mask-fit over a wide range of facial structures sizes has been established via the NIOSH facial anthropometric database model. In accordance with some aspects, one size fits most individuals (based on NIOSH anthropometric measurement). The NIOSH small size is suitable for children, while the medium and large sizes are suitable for teens and adults. Some embodiments, have an over molded Aluminum wire around the periphery for further size/shape adjustment to a particular face shape. Also, self-sealing and seal testing features in the mask assure the user of a proper seal. In embodiments, elastomeric seal functionality is used, which further improves the contact between the seal and the facial structures of a wearer. In various embodiments, the masks of the present specification use Lanyard type single-loop head strap built to ASTM Standard F3407. The Lanyard type, single-loop behind the head strap meets the NIOSH N-95 standards, has ease of wearability of the air-loop strap without the discomfort of air-loop straps and allows the mask to hang-around wearer's neck when not in use for easy access when needed.

In various embodiments, the masks of the present specification have a modular design that allows for addition of electronic features such as sanitization (UV/Ionization). In some embodiments, the modular design allows for application of low-cost, bipolar ionization for destructive filtration into the face mask.

The masks of the present specification have 1) high filtration efficiency providing an N-95 or greater protection, 2) comfortable fit and flexibility of the mask materials and form factor that fits most head sizes without the need for formal “fit-testing”, and 3) the overall utility afforded by its reusable design, replaceable filters, and see-through material creating an affordable mask which is environmentally responsible. Specifically, the masks overcome the barrier to fit by use of a flexible seal material and overcome the barrier to comfort by increased contact surface area resulting in the reduction of skin pressure points, flexible mask body, and high-flow filters. The mask addresses many of the significant shortcomings of prior art designs, including low efficacy, obstruction of facial features, poor medium-and long-term wearability, and reprocessing challenges. Further, mask body of the present specification has a durometer such that the in and out motion of the soft mask body with respiration tells the wearer that a proper seal has been established.

In accordance with some aspects, the filters (of the masks of the present specification) are non-biodegradable so by reducing the filter size and increasing filter use duration, the waste foot-print of the masks is reduced. The efficiency and the life of the filters can be further improved by adding bipolar ionization to the mask.

Embodiments of the present specification offer several advantages over some characteristics of the masks of the prior art. Existing configurations of masks are fraught with communication and security issues, since they often enable poor visibility of a wearer's face, leading to inability in identification of the wearer. Current configurations also interfere with the wearer's hearing and learning abilities. In contrast, the masks of the present specification are optically clear with filters that offset laterally, thereby creating a zone of visibility around the user's mouth and allowing for facial recognition.

Currently used mask configurations, such as, for example, masks made of cloth or non-woven fabric, have low filtration efficacy. In contrast, the masks of the present specification, in some embodiments, use N 95+/N 99+ filters.

Prior art masks present significant reprocessing challenges, for example, cloth masks need to be laundered and N95 respirators are meant to be disposed. In contrast, the masks of the present specification are reusable and have replaceable filters. In some embodiments, a packaged mask of the present specification with 26 filters lasts for six months with weekly filter changes. In some embodiments, the masks are washable with soap and water, and filters are configured to be changed weekly so that the masks can be used continuously for six months before being discarded.

Fit testing is needed for prior art N95 respirators, which is often unsuitable for consumer use. Further many individuals fail the fit testing. In contrast, the masks of the present specification have an elastomeric body that conforms to a wide range face types. Mask design features such as, and not limited to, bladder soft seal, malleable perimeter wire, flexible mask body, strap attachment design, allows for the masks to fit to NIOSH ASTM standard F3407 in face lengths ranging from 98.5 mm-138.5 mm and face widths ranging from 120.5 mm-158.5 mm in more than 50% of users without need for sizing using a formal fit test.

Prior art masks are fraught with issues of physical discomfort especially in humid environments, breathing difficulty, and contact dermatitis. In contrast, masks of the present specification have high-efficiency filters and broader skin contact area allowing for a better and more comfortable seal, low air flow resistance of 22 Pa. Also, use of FDA grade silicone in embodiments of the present specification eliminates contact dermatitis making it comfortable to wear over long period of time.

### PHYSICAL CHARACTERISTICS

In various embodiments, the masks described in the present specification have the following characteristics with respect to various mask elements.

### Mask Body

The body of various embodiments of the mask of present specification is configured to be optically clear. The optical clarity offered by the masks of present specification enable phone or digital facial image recognition and facial feature visibility. The optical clarity of the mask body is achieved by using material such as LSR-4350 Silicone 50A durometer, Elkem or equivalent, or Dow Corning Optical grade M S 1002 silicone or equivalent. The mask body is finished with polished A 2. In embodiments, a thickness of the mask body ranges within 0.050 +/−10%.

In embodiments, the mask body is further configured with an anti-fog surfactant or a polycarbonate insert. Materials used for the surfactant include SP-3300, 2 pph or equivalent. Additionally, the mask body of the present specification offers features such as anti-glare, is non-sticky so that it does not attract hair or dirt, is configured using odorless materials, and supports anti-microbial coating. The mask body includes an integrated sealed perimeter using integrated wire(s) to sustain 500 adjustments (for example, single wire design vs. 3 wires across nose, cheek bones and across jawline). Material used for the wire(s) may include stainless steel or aluminum. Further, in embodiments integrated strap anchors are incorporated with the mask body, which enable user-performed strap changes. Additionally, the strap attachment allows for various strap options including, but not limited to, two strap, bungie strap, and ear loop. Mask bodies of the present specification are capable of functioning for six months of general use and wash. The embodiments prevent fogging of eye glasses of the wearer, making it easy to wear with eye glasses.

### Filter Ports

Embodiments of the present specification enable use of one or more filter ports. The port location(s) are optimized for breathability and filter size while not covering frontal facial features so as to enable phone/digital image recognition. Filters are secured in place while in use and are easy to change. For example, in one embodiment, filters are held in place using magnets, under 1.5 lb pull force, as described with respect to FIGS. 24E and 24F. Surface area of the filters is maximized for breathability. In some embodiments, the surface area is >5 inch². In embodiment, the support structure on the mask, such as web-like structure, that is configured to secure the filter in place, is reduced. The filter embodiments of present specification are capable of functioning for at least six months of general use and wash. Further, in embodiments, an airtight seal is provided with filter port cover.

Filter port covers secure each filter in place while the mask is in use. In embodiments, material for filter port covers include biocompatible plastics and magnets. Further, the embodiments allow for easy replacement of filters by a user. The filters can be replaced by the user with minimal handling. The embodiments allow sustaining approximately 50 filter replacements by the user. In embodiments, filter cover is positioned on the inside of the mask, to minimize protrusion from mask surface. In embodiments, the web with the filter is minimal to maximize airflow. In embodiments an airtight seal is provided with the filter port.

### Air Content Monitoring and Control

Air molecule-specific gas sensors are incorporated with the masks of the present specification, to measure content of any combination of air elements such as nitrogen, oxygen, carbon dioxide, hydrogen, and other. In embodiments, a moisture sensor is incorporated to measure air humidity. In embodiments, one or more sensors are incorporated outside of mask body or mask ports to measure incoming ambient air. In embodiments, one or more sensors are incorporated inside mask surface or mask ports to measure exhaled air and/or filtered air. The one or more sensors are electronically powered and connected. The one or more sensors comprise thin film substrates, solid state substrates, or other types of substrates. The one or more sensors are configured to be connected through wire or wireless means, such as via Bluetooth, to a remote digital device, such as a smartphone, to report measurements.

In embodiments, air content is limited for a specific molecule or a combination of molecules thereof by employing air molecule-specific filtration techniques at the inlet and/or outlet ports. The air molecule-specific filtration can include non-electrical filtration mechanisms or those requiring electronics. The electronic or non-electronic air control mechanism can be wearable directly on the mask of the present specification, or connected to the mask and worn elsewhere on the body.

In embodiments, fresh air content is limited by feeding expired air out a plurality of output ports, and fresh air is routed directly to a plurality of input ports, where both types of ports are valve-controlled for desired flow directionality. In embodiments, internal humidity within the mask of the present specification is limited by incorporating a desiccant inside the mask. The air control is augmented by controlling flow of ambient air or filtered ambient air through a plurality of input ports that are valve-controlled for desired directionality. In further embodiments, the control mechanism is augmented by incorporating fans to assist desired air flow(s). In embodiments, a microcontroller is used for effecting the control. In embodiments, the microcontroller is used to have a closed-loop control over specific air molecule(s) percentage to be breathed in. In embodiments, closed-loop control can set a percentage or modulate the percentage anywhere between 0-100% over time.

The mask embodiments of the present specification can be used in several applications. For example, to provide an oxygen-enriched environment for people with respiratory diseases such as COPD; in a lower humidity environment for people with respiratory diseases such as COPD; in an air assisted environment for people with respiratory diseases such as COPD; in an oxygen-starved environment for people training such as in athletics, marathon, and military, to simulate high altitude; to provide a stable air environment to prevent asthma; to provide a stable environment to prevent allergic reactions to airborne allergens. Further, the embodiments of mask of present specification may be substituted for N-95 applications in medicine and for consumers. The mask may also assist in breathing.

### Breathing Quality Monitoring

Embodiments of the present specification are used for monitoring quality of breathing of the wearer. Air molecule-specific gas sensors are incorporated in the mask to measure content of exhaled air. Further, pressure sensors are incorporated to measure strength of exhaled breathing and also respiration rate. In embodiments, flow sensors are incorporated to measure speed of exhaled and inhaled breathing (and calculate tidal volume) and also respiration rate. In embodiments, the sensors are electronically powered and connected. Thin film substrates, solid state substrates, or other types of substrates are used to configure the sensors. In embodiments, the sensors communicate over wire or wireless connections such as Bluetooth to communicate with remote devices such as a smartphone, to report measurements.

Breathing quality is monitored to monitor efficacy of breathing, determine severity (acute and/or chronic) of breathing disorders, monitor consumption of respiratory medications by measuring exhaled air content, tidal volume, breathing pressure, and breathing rate. In some embodiments, the monitoring enables detection of cancer progression by measuring exhaled air content. In some embodiments, alcohol level is detected by embodiments of the present specification. The various monitored parameters may also aid the wearer or a caretaker with assisting to sleep.

### Sleep Apnea Diagnosis, Monitoring, and Control

Embodiments of the present specification are used to diagnose, monitor, and control breathing related disorders such as sleep apnea. In embodiments, pressure sensors are incorporated within a mask chamber of the present specification, to detect for possible air leaks (mask not seated properly). Proper fitting without air leaks, allows for accurate monitoring of respiration rate. In embodiments, skin impedance sensors are incorporated on the silicone seal to detect for possible air leaks (in cases where mask is not seated properly). In embodiments, flow sensors are incorporated to measure speed of exhaled and inhaled breathing (and calculate tidal volume) as well as monitor respiration rate (and detect apnea events). In embodiments, an electronic microphone is incorporated to monitor respiration rate as well snoring events (and detect apnea events). In embodiments, at least two electrodes are incorporated to monitor heart rate. A 3-axis accelerometer is also incorporated in some embodiments to monitor sleeping position and activity. The monitors/sensors are electronically powered and connected using thin film substrates, solid state substrates, or others, and are enabled for wire or wireless communication with a remote device such as a smartphone.

In embodiments, sleep apnea or other respiration disorders are controlled by the present specification such as by incorporating a fan to assist breathing during sleep, as well as to maintain a minimum pressure within the sealed mask. In embodiments, the control is enabled by a microcontroller. The microcontroller may have a closed-loop control over the fan, using any or all of the above sensors as inputs. A closed-loop control can turn the fan on, off, or modulate its speed.

Monitoring and controlling respiratory disorders such as sleep apnea, by means of the embodiments of the present specification, provide benefits of wearer's sleep quality assessment without need for a sleep lab. Further, wearer's likelihood of having a disorder such as sleep apnea likelihood assessment is determined without need for a sleep lab. The embodiments of present specification are much more cost effective and economical than a sleep lab. The embodiments offer a CPAP alternative to treating sleep apnea and monitoring sleep quality (smaller device, not tethered, no cleaning easier to travel).

The above examples are merely illustrative of the many applications of the systems, methods, and apparatuses of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

1. A device adapted to generate a flow of air around a user's head, comprising:
a band configured to fit on, and encircle, at least a portion of a user's head or neck such that the user's eyes, mouth, and nose remain uncovered by the device;
at least one air inlet port coupled to the band;
a pump positioned inside the band and configured to draw air through the plurality of air inlet ports;
an air filtration system positioned inside the band and in fluid communication with the air inlet ports; and
a plurality of air outlet ports in fluid communication with the air filtration system, wherein the air outlet ports are configured to direct an outflow of air over at least the user's nose and mouth.

2. The device of claim 1, wherein, in operation, the outflow of air generates a clean air bubble surrounding the user's nose and/or mouth and having a volume of at least one liter.

3. The device of claim 1, wherein the pump provides a clean air delivery rate (CADR) ranging from 10 L/minute to 500 L/minute.

4. The device of claim 1, wherein the pump operates at a pressure less than or equal to 50 psi.

5. The device of claim 1, wherein the air filtration system comprises one or more of: an ultraviolet destruction mechanism ranging from 200 nm to 260 nm, photochemical oxidation (PCO) or photo-electrochemical oxidation (PECO) destruction components, or negative ion, bipolar, or plasma ion antimicrobial destruction systems.

6. The device of claim 1, wherein the air filtration system comprises one or more particulate filters and one or more antimicrobial filters.

7. The device of claim 6, wherein the antimicrobial filters comprise one of a hydrophobic PES, PTFE, glass microfiber membrane, or HEPA filter housed in a polypropylene or styrene housing.

8. The device of claim 7, wherein each of the antimicrobial filters ranges in size from 5 mm to 100 mm having a pore size ranging from 0.1 μm to 100 μm.

9. The device of claim 6, wherein the particulate filters comprise one of a hydrophilic PES, PTFE, glass microfiber membrane, electrostatic filter, or nylon filter housed in a polypropylene housing.

10. The device of claim 9, wherein each of the particulate filters ranges in size from 5 mm to 100 mm having pore sizes ranging from 0.1 μm to 100 μm.

11. The device of claim 1, wherein the air filtration system comprises one or more activated charcoal or carbon filter for filtering harmful ions, ozone, and/or volatile organic compounds (VOCs).

12. The device of claim 1, wherein the air filtration system comprises at least one of a low efficiency filter having a MERV rating of less than or equal to 11 or a high efficiency filter having a MERV rating of greater than or equal to 11.

13. The device of claim 1, wherein the air filtration system comprises at least one far Ultraviolet (UV-C) filter having a wavelength ranging from 207-280 nm.

14. The device of claim 1, wherein the band comprises a structure positioned at a center portion of the band, the structure housing the plurality of air outlet ports for creating a flow of air over the user's face, wherein the structure is adapted to be tilted for directing the flow of air to create an air shield around the user's head by directing the flow of air over the user's face.

15. The device of claim 1, wherein the band is configured to be placed on the user's head and comprises a first end and a second end opposite the first end, wherein the first end and the second end each comprise at least some of the air outlet ports for creating a flow of air over the user's face to create an air shield around the user's head.

16. The device of claim 1, wherein the band is configured to be placed on the user's head and has a length defined by a first end and a second end opposite the first end, wherein the plurality of air outlet ports for creating a flow of air over the user's face to create an air shield around the user's head by directing the flow of air over the user's face is positioned in the length.

17. The device of claim 1, wherein the band is configured to be placed on the user's neck and has a length defined by a first end and a second end opposite the first end, wherein the plurality of air outlet ports for creating a flow of air upwards and away the user's face to create an air shield around the user's head are positioned in the length.

18. The device of claim 1, wherein the band comprises a frame for fitting around the user's eyes and nose, wherein the frame comprises a plurality of air inlets for receiving purified air and a plurality of air outlets for delivering purified air and creating an air shield around the user's head by directing the flow of air over the user's face.

19. The device of claim 18, wherein the flow of purified air creates a volume of clean air around the user's nose, wherein the volume meets 50% or more of the user's peak flow requirement during inhalation.

20. The device of claim 1, having a noise level of less than 65 decibel during operation.

21. The device of claim 1, wherein a total weight of the device is less than 5 lbs.
